(19) United States
(12) Patent Application Publication
(10) Pub. No.: US 2016/0349221 A1
(43) Pub. Date: Dec. 1, 2016

(54) METHODS FOR MASS SPECTROMETRIC QUANTITATION OF ANALYTES EXTRACTED FROM A MICROSAMPLING DEVICE

(71) Applicant: QUEST DIAGNOSTICS INVESTMENTS LLC

(72) Inventors: Scott Goldman, Laguna Niguel, CA (US); Mildred Goldman, Laguna Niguel, CA (US); Leslie Edinboro, Glen Allen, VA (US); Diana Tran, Garden Grove, CA (US); Julia Addiss, Carlsbad, CA (US); Darren Weber, Rancho Santa Margarita, CA (US); Porus Mistry, Irvine, CA (US); Nigel Clarke, Vista, CA (US)

(73) Assignee: Quest Diagnostics Investments LLC, Madison, NJ (US)

(21) Appl. No.: 15/167,866

(22) Filed: May 27, 2016

Related U.S. Application Data
(60) Provisional application No. 62/167,164, filed on May 27, 2015.

(51) Int. Cl. G01N 30/72; G01N 33/49; G01N 33/493; H01J 49/00; H01J 49/14

(52) U.S. Cl. CPC G01N 30/7233; H01J 49/0031; H01J 49/145; H01J 49/005; G01N 33/493; G01N 33/49

(57) ABSTRACT

Mass spectrometric methods are described for determining the amount of analyte in a sample collected by a microsampling device. Provided herein are methods directed to quantitating the amount of an analyte in a sample by extracting an analyte from a sample collected by a microsampling device, purifying the sample by liquid chromatography, ionizing the analyte to generate one or more ions detectable by mass spectrometry; and determining the amount of the one or more ions by mass spectrometry. The amount of analyte in the sample is related to the amount of analyte in the patient.

### CROSS REFERENCE TO RELATED PATENT APPLICATIONS

This application claims priority to U.S. Application Ser. No. 62/167,164, filed May 27, 2015, each of which is incorporated herein by reference in its entirety.

### BACKGROUND OF THE INVENTION

Mass spectrometric quantitation of analytes from patients requires collection of fluid samples in relatively large quantities. Such samples require refrigeration in dry ice or freezing for transport, which is expensive and burdensome on personnel handling the samples. Also the fluid samples may be considered a biohazard that requires a special transport method.

Collection of patient samples using dried blood spot cards requires less volume than the fluid collection described above. Dried blood spot specimens are collected by applying a few drops of blood obtained from pricking the heel or finger and blotted onto filter paper, which then is hole punched for extraction and analysis. However, dried blood spot cards present issues of inconsistency and variability in quantitation due to inherent separation of the red blood cells and serum that occurs after placement of blood on filter paper. Also, variability of the location of the hole punch area to be extracted and analyzed could significantly affect the quantitation results.

A reliable and accurate method for mass spectrometric quantitation of analytes is needed.

### SUMMARY OF THE INVENTION

In one aspect, provided herein are methods for mass spectrometric quantitation of analytes collected and extracted from a microsampling device.

In certain embodiments, the methods provided herein are directed to quantitating the amount of an analyte in a sample comprising (a) extracting an analyte from a sample collected by a microsampling device; (b) ionizing the analyte to generate one or more ions detectable by mass spectrometry; and (c) determining the amount of the one or more ions by mass spectrometry. In some embodiments, the amount of the one or more ions determined is used to determine the amount of analyte in the sample. In some embodiments, the amount of analyte in the sample is related to the amount of analyte in the patient.

In certain embodiments, the methods provided herein are directed to quantitating the amount of an analyte in a capillary blood sample comprising (a) extracting an analyte from a capillary blood sample collected by a microsampling device; (b) ionizing the analyte to generate one or more ions detectable by mass spectrometry; and (c) determining the amount of the one or more ions by mass spectrometry. In some embodiments, the amount of the one or more ions determined is used to determine the amount of analyte in the sample. In some embodiments, the amount of analyte in the sample is related to the amount of analyte in the patient.

In some embodiments, the capillary blood is collected by microsampling device. In some embodiments, the capillary blood is not collected by a dried blood spot.

In some embodiments, the methods provided herein comprise purifying the samples prior to mass spectrometry. In some embodiments, the methods comprise purifying the samples using liquid chromatography. In some embodiments, liquid chromatography comprise high performance liquid chromatography (HPLC) or high turbulence liquid chromatograph (HTLC). In some embodiments, the methods comprise subjecting a sample to solid phase extraction (SPE). In some embodiments, the methods comprise subjecting a sample to reverse phase analytical column.

In some embodiments, the methods provided herein are directed to quantitating the amount of an analyte in a sample comprising (a) extracting an analyte from a sample collected by a microsampling device, (b) purifying the sample by liquid chromatography, (c) ionizing the analyte to generate one or more ions detectable by mass spectrometry; and (d) determining the amount of the one or more ions by mass spectrometry. In some embodiments, the amount of the one or more ions determined is used to determine the amount of analyte in the sample. In some embodiments, the amount of analyte in the sample is related to the amount of analyte in the patient.

In some embodiments, mass spectrometry comprises tandem mass spectrometry. In some embodiments, mass spectrometry is high resolution mass spectrometry. In some embodiments, mass spectrometry is high resolution/high accuracy mass spectrometry.

In some embodiments, ionization is by atmospheric pressure chemical ionization (APCI). In some embodiments, ionization is by electrospray ionization (ESI). In some embodiments, said ionization is in positive ion mode. In some embodiments, said ionization is in negative ion mode.

In some embodiments, the microsampling device containing the sample is placed in a 96-well plate. In some embodiments, the microsampling device containing the sample is placed in a 96-rack. In some embodiments, automation places the 96-rack into a 96-well plate. In some embodiments, the automation is HAMILTON® automation.

In some embodiments, the methods provided herein comprise adding internal standards to the sample. In some embodiments, the internal standard is labeled. In some embodiments, the internal standard is deuterated or isotopically labeled. In some embodiments, the internal standard is added with extraction buffer. In some embodiments, the microsampling device is pre-soaked with internal standard and dried.

In some embodiments, the extracting step comprises adding an extraction buffer to the sample collected by a microsampling device. In some embodiments, the extracting step comprises placing the microsampling device containing the sample into a 96-well plate containing an extraction solvent. In some embodiments, the extraction step is automated. In some embodiments, 96-well plate is vortexed and then the absorbent tips of the microsampling device are removed. In some embodiments, the extracting step comprises drying down under nitrogen. In some embodiments, the extracting step comprises reconstituting the sample into solution. In some embodiments, the reconstitution comprises adding aqueous acid or organic solution or both to the sample. In some embodiments, the reconstituted solution is filtered.

In some embodiments, the methods provided herein comprise high-throughput automation of extraction and mass spectrometric analysis of multiple samples at the same time. In some embodiments, the methods provided herein comprise using an apparatus that enables automation of extraction and mass spectrometric analysis of multiple samples at the same time. In some embodiments, an apparatus that enables automation comprise a microsampling device. In some embodiments, the microsampling device is configured in a high-throughput apparatus.

In some embodiments, the extracted sample is injected into a mass spectrometric system. In some embodiments, the extracted sample is injected into liquid chromatography. In some embodiments, the extraction and mass spectrometry steps are performed in an on-line fashion to allow for automated sample analysis. In some embodiments, the extraction, purification, and mass spectrometry steps are performed in an on-line fashion to allow for automated sample analysis.

In some embodiments, the analyte is underivatized.

In some embodiments, the sample collected by the microsampling device does not require sample processing.

In some embodiments, the sample collected by the microsampling device is whole blood. In some embodiments, the sample collected by the microsampling device is urine. In some embodiments, the sample collected by the microsampling device is saliva. In some embodiments, the sample collected by the microsampling device is serum or plasma.

In some embodiments, the microsampling device comprises an absorbent tip that collects the sample. In some embodiments, the sample collected by the microsampling device absorbs a fixed volume of patient fluids. In some embodiments the patient fluid is capillary blood. In some embodiments, the sample collected by the microsampling device has a volume of less than or equal to 150 μL. In some embodiments, the sample collected by the microsampling device has a volume of less than or equal to 100 μL. In some embodiments, the sample collected by the microsampling device has a volume of less than or equal to 50 μL. In some embodiments, the sample collected by the microsampling device has a volume of between 5 μL and 150 μL, inclusive. In some embodiments, the sample collected by the microsampling device has a volume of between 10 μL and 100 μL, inclusive. In some embodiments, the sample collected by the microsampling device has a volume of about 10 μL. In some embodiments, the sample collected by the microsampling device has a volume of about 15 μL. In some embodiments, the sample collected by the microsampling device has a volume of about 20 μL. In some embodiments, the sample collected by the microsampling device has a volume of about 30 μL. In some embodiments, the sample collected by the microsampling device has a volume of about 50 μL. In some embodiments, the sample collected by the microsampling device has a volume of about 100 μL. In some embodiments, the sample collected by the microsampling device absorbs a fixed volume of blood, regardless of the amount of hematocrit.

In certain embodiments, the methods provided herein are directed to quantitating the amount of an analyte in a low volume of sample. In some embodiments, the methods provided herein are directed to quantitating the amount of an analyte in a sample comprising (a) extracting an analyte from a sample of less than or equal to 100 μL; (b) ionizing the analyte to generate one or more ions detectable by mass spectrometry; and (c) determining the amount of the one or more ions by mass spectrometry. In some embodiments, the amount of the one or more ions determined is used to determine the amount of analyte in the sample. In some embodiments, the amount of analyte in the sample is related to the amount of analyte in the patient.

In some embodiments, the sample is capillary blood sample. In some embodiments, the sample is not venous blood sample.

In some embodiments, the methods provided herein are directed to quantitating the amount of an analyte in a low volume of capillary blood sample. In some embodiments, the methods provided herein are directed to quantitating the amount of an analyte in a sample comprising (a) extracting an analyte from capillary blood sample of less than or equal to 100 μL; (b) purifying the sample by liquid chromatography; (c) ionizing the analyte to generate one or more ions detectable by mass spectrometry; and (d) determining the amount of the one or more ions by mass spectrometry. In some embodiments, the amount of the one or more ions determined is used to determine the amount of analyte in the capillary blood sample. In some embodiments, the amount of analyte in the sample is related to the amount of analyte in the patient.

In some embodiments, the methods comprise extracting an analyte from a sample of less than or equal to 50 μL. In some embodiments, the methods comprise extracting an analyte from a sample of less than or equal to 30 μL. In some embodiments, the methods comprise extracting an analyte from a sample of less than or equal to 20 μL. In some embodiments, the methods comprise extracting an analyte from a sample of less than or equal to 15 μL. In some embodiments, the methods comprise extracting an analyte from a sample of less than or equal to 10 μL.

In some embodiments, the sample collected by the microsampling device can be transported without refrigeration or freezing. In some embodiments, the sample collected by the microsampling device can be transported without dry ice. In some embodiments, the sample collected by the microsampling device can be transported at room temperature. In some embodiments, the sample collected by the microsampling device can be transported without biohazard concerns.

In some embodiments, the sample collected by the microsampling device requires little training for collection. In some embodiments, the sample collected by the microsampling device can be collected anywhere. In some embodiments, the sample collected by the microsampling device can be dried at ambient temperature for shipping.

In some embodiments, the microsampling device comprises apparatus that enables automation of extraction and mass spectrometric analysis. In some embodiments, the microsampling device comprises apparatus that enables high-throughput automation of extraction and mass spectrometric analysis of multiple samples at the same time. In some embodiments, the microsampling device is a MITRA® tip. In some embodiments, the microsampling device is encased in a cartridge designed for automation of extraction and mass spectrometric analysis.

In some embodiments, the methods further comprise collecting the sample with a microsampling device. In some embodiments, the collecting step comprises performing a finger prick and applying an absorbent tip of the microsampling device to the blood. In some embodiments, the collecting step comprises applying an absorbent tip in the urine or saliva of the patient. In some embodiments, the sample collected in the microsampling device is air dried. In some embodiments, the sample collected in the microsampling device is air dried for 1 to 2 hours prior to transport.

In some embodiments, the analyte is a steroid. In some embodiments, the steroid is cortisol, cortisone, progesterone, 17-hydroxyprogesterone, androstenedione, testosterone, dehydroepiandrosterone, corticosterone, deoxycorticosterone, 11-deoxycortisol, pregnenolone, 17-hydroxypregnenolone, 18-hydroxycorticosterone, or 21-deoxycortisol. In some embodiments, the analyte is a steroid in a steroid panel for diagnosing congenital adrenal hyperplasia (CAH). In some embodiments, the steroid is selected from the group consisting of cortisol, cortisone, progesterone, 17-hydroxyprogesterone, androstenedione, testosterone, dehydroepiandrosterone, corticosterone, deoxycorticosterone, 11-deoxycortisol, pregnenolone, 17-hydroxypregnenolone, 18-hydroxycorticosterone, and 21-deoxycortisol. In some embodiments, the steroid is 25-hydroxyvitamin D₂ or 25-hydroxyvitamin D₃.

In some embodiments, one or more ions comprise a cortisone precursor ion with a mass to charge ratio (m/z) of 361.4±0.5. In some embodiments, one or more ions comprise one or more fragment ions with a mass to charge ratio (m/z) of 121.2±0.5 or 163.2±0.5. In some embodiments, one or more ions comprise a cortisol precursor ion with a mass to charge ratio (m/z) of 363.4±0.5. In some embodiments, one or more ions comprise one or more fragment ions with a mass to charge ratio (m/z) of 121.1±0.5 or 267.2±0.5. In some embodiments, one or more ions comprise a 21-deoxycortisol precursor ion with a mass to charge ratio (m/z) of 347.3±0.5. In some embodiments, one or more ions comprise one or more fragment ions with a mass to charge ratio (m/z) of 121.1±0.5 or 269.2±0.5. In some embodiments, one or more ions comprise a coticosterone precursor ion with a mass to charge ratio (m/z) of 347.4±0.5. In some embodiments, one or more ions comprise one or more fragment ions with a mass to charge ratio (m/z) of 121.1±0.5 or 311.3±0.5. In some embodiments, one or more ions comprise a 11-deoxycortisol precursor ion with a mass to charge ratio (m/z) of 347.4±0.5. In some embodiments, one or more ions comprise one or more fragment ions with a mass to charge ratio (m/z) of 97.1±0.5 or 109.1±0.5. In some embodiments, one or more ions comprise an androstenedione precursor ion with a mass to charge ratio (m/z) of 287.4±0.5. In some embodiments, one or more ions comprise one or more fragment ions with a mass to charge ratio (m/z) of 97.1±0.5 or 109.1±0.5. In some embodiments, one or more ions comprise a 11-deoxycorticosterone precursor ion with a mass to charge ratio (m/z) of 331.4±0.5. In some embodiments, one or more ions comprise one or more fragment ions with a mass to charge ratio (m/z) of 97.1±0.5 or 109.1±0.5. In some embodiments, one or more ions comprise a testosterone precursor ion with a mass to charge ratio (m/z) of 289.4±0.5. In some embodiments, one or more ions comprise one or more fragment ions with a mass to charge ratio (m/z) of 97.1±0.5 or 109.1±0.5. In some embodiments, one or more ions comprise a 17-hydroxyprogesterone precursor ion with a mass to charge ratio (m/z) of 331.4±0.5. In some embodiments, one or more ions comprise one or more fragment ions with a mass to charge ratio (m/z) of 97.1±0.5 or 109.1±0.5. In some embodiments, one or more ions comprise a progesterone precursor ion with a mass to charge ratio (m/z) of 315.3±0.5. In some embodiments, one or more ions comprise one or more fragment ions with a mass to charge ratio (m/z) of 97.1±0.5 or 109.1±0.5. In some embodiments, one or more ions comprise a cortisone-d7 precursor ion with a mass to charge ratio (m/z) of 369.4±0.5. In some embodiments, one or more ions comprise one or more fragment ions with a mass to charge ratio (m/z) of 169.2±0.5. In some embodiments, one or more ions comprise a cortisol-d4 precursor ion with a mass to charge ratio (m/z) of 367.4±0.5. In some embodiments, one or more ions comprise one or more fragment ions with a mass to charge ratio (m/z) of 121.0±0.5. In some embodiments, one or more ions comprise a corticosterone-d4 precursor ion with a mass to charge ratio (m/z) of 351.1±0.5. In some embodiments, one or more ions comprise one or more fragment ions with a mass to charge ratio (m/z) of 121.1±0.5. In some embodiments, one or more ions comprise a 11-deoxycortisol-13C3 precursor ion with a mass to charge ratio (m/z) of 350.4±0.5. In some embodiments, one or more ions comprise one or more fragment ions with a mass to charge ratio (m/z) of 100.1±0.5. In some embodiments, one or more ions comprise an androstendione-13C3 precursor ion with a mass to charge ratio (m/z) of 290.4±0.5. In some embodiments, one or more ions comprise one or more fragment ions with a mass to charge ratio (m/z) of 100.1±0.5. In some embodiments, one or more ions comprise a testosterone-13C3 precursor ion with a mass to charge ratio (m/z) of 292.4±0.5. In some embodiments, one or more ions comprise one or more fragment ions with a mass to charge ratio (m/z) of 112.1±0.5. In some embodiments, one or more ions comprise a 17-hydroxyprogesterone-13C3 precursor ion with a mass to charge ratio (m/z) of 334.3±0.5. In some embodiments, one or more ions comprise one or more fragment ions with a mass to charge ratio (m/z) of 100.0±0.5. In some embodiments, one or more ions comprise a progesterone-13C3 precursor ion with a mass to charge ratio (m/z) of 318.5±0.5. In some embodiments, one or more ions comprise one or more fragment ions with a mass to charge ratio (m/z) of 100.1±0.5.

In some embodiments, the analyte is an opiate. In some embodiments, the opiate is cis-tramadol, O-desmethyl tramadol, tapentadol, N-desmethyltapentadol, codeine, morphine, oxymorphone, norhydrocodone, oxycodone, noroxycodone, hydromorphone, hydrocodone, buprenorphine, norbuprenorphine, fentanyl, norfentanyl, 6-monoacetylmorphine (6-MAM), methadone, dihydrocodeine, naloxone, naltrexone, 6β-naltrexol, nalorphine, nalbuphine, or 2-ethylidene-1,5-dimethyl-3,3-diphenylpyrrolidine (EDDP). In some embodiments, the opiate is selected from the group consisting of cis-tramadol, O-desmethyl tramadol, tapentadol, N-desmethyltapentadol, codeine, morphine, oxymorphone, norhydrocodone, oxycodone, noroxycodone, hydromorphone, hydrocodone, buprenorphine, norbuprenorphine, fentanyl, norfentanyl, 6-monoacetylmorphine (6-MAM), methadone, dihydrocodeine, naloxone, naltrexone, 6β-naltrexol, nalorphine, nalbuphine, and 2-ethylidene-1,5-dimethyl-3,3-diphenylpyrrolidine (EDDP). In some embodiments, the opiate is extracted from a whole blood, salive, or urine sample.

In some embodiments, the analyte is a benzodiazepine. In some embodiments, the benzodiazepine is oxazepam, temazepam, lorazepam, nordiazepam, diazepam, chlordiazepoxide, triazolam, midazolam, alprazolam, clonazepam, bromazepam, clobazam, nitrazepam, phenazepam, prazepam, medazepam, flunitrazepam, or flurazepam. In some embodiments, the benzodiazepine is selected from the group consisting of oxazepam, temazepam, lorazepam, nordiazepam, diazepam, chlordiazepoxide, triazolam, midazolam, alprazolam, clonazepam, bromazepam, clobazam, nitrazepam, phenazepam, prazepam, medazepam, flunitrazepam, and flurazepam. In some embodiments, the benzodiazepine is extracted from a whole blood or urine sample.

In some embodiments, one or more ions comprise a bromazepam precursor ion with a mass to charge ratio (m/z) of 316±0.5. In some embodiments, one or more ions comprise one or more fragment ions with a mass to charge ratio (m/z) of 214±0.5 or 270±0.5. In some embodiments, one or more ions comprise an oxazepam precursor ion with a mass to charge ratio (m/z) of 287±0.5. In some embodiments, one or more ions comprise one or more fragment ions with a mass to charge ratio (m/z) of 104±0.5 or 241±0.5. In some embodiments, one or more ions comprise an clobazam precursor ion with a mass to charge ratio (m/z) of 300±0.5. In some embodiments, one or more ions comprise one or more fragment ions with a mass to charge ratio (m/z) of 224±0.5 or 259±0.5. In some embodiments, one or more ions comprise a nitrazepam precursor ion with a mass to charge ratio (m/z) of 282±0.5. In some embodiments, one or more ions comprise one or more fragment ions with a mass to charge ratio (m/z) of 180±0.5 or 236±0.5. In some embodiments, one or more ions comprise an alprazolam precursor ion with a mass to charge ratio (m/z) of 309.1±0.5. In some embodiments, one or more ions comprise one or more fragment ions with a mass to charge ratio (m/z) of 165±0.5 or 280.9±0.5. In some embodiments, one or more ions comprise an triazolam precursor ion with a mass to charge ratio (m/z) of 343±0.5. In some embodiments, one or more ions comprise one or more fragment ions with a mass to charge ratio (m/z) of 206±0.5 or 308±0.5. In some embodiments, one or more ions comprise a clonazepam precursor ion with a mass to charge ratio (m/z) of 316±0.5. In some embodiments, one or more ions comprise one or more fragment ions with a mass to charge ratio (m/z) of 214±0.5 or 270±0.5. In some embodiments, one or more ions comprise a flurazepam precursor ion with a mass to charge ratio (m/z) of 388±0.5. In some embodiments, one or more ions comprise one or more fragment ions with a mass to charge ratio (m/z) of 287.9±0.5 or 315±0.5. In some embodiments, one or more ions comprise a lorazepam precursor ion with a mass to charge ratio (m/z) of 321±0.5. In some embodiments, one or more ions comprise one or more fragment ions with a mass to charge ratio (m/z) of 229.1±0.5 or 331±0.5. In some embodiments, one or more ions comprise a flunitrazepam precursor ion with a mass to charge ratio (m/z) of 314±0.5. In some embodiments, one or more ions comprise one or more fragment ions with a mass to charge ratio (m/z) of 211±0.5 or 268±0.5. In some embodiments, one or more ions comprise a temazepam precursor ion with a mass to charge ratio (m/z) of 301.1±0.5. In some embodiments, one or more ions comprise one or more fragment ions with a mass to charge ratio (m/z) of 177±0.5 or 255±0.5. In some embodiments, one or more ions comprise a midazolam precursor ion with a mass to charge ratio (m/z) of 326±0.5. In some embodiments, one or more ions comprise one or more fragment ions with a mass to charge ratio (m/z) of 129±0.5 or 244±0.5. In some embodiments, one or more ions comprise an nordiazepam precursor ion with a mass to charge ratio (m/z) of 271±0.5. In some embodiments, one or more ions comprise one or more fragment ions with a mass to charge ratio (m/z) of 139.8±0.5 or 165±0.5. In some embodiments, one or more ions comprise an phenazepam precursor ion with a mass to charge ratio (m/z) of 351±0.5. In some embodiments, one or more ions comprise one or more fragment ions with a mass to charge ratio (m/z) of 185.9±0.5 or 206±0.5. In some embodiments, one or more ions comprise a chlordiazepam precursor ion with a mass to charge ratio (m/z) of 301±0.5. In some embodiments, one or more ions comprise one or more fragment ions with a mass to charge ratio (m/z) of 259±0.5 or 224±0.5. In some embodiments, one or more ions comprise a diazepam precursor ion with a mass to charge ratio (m/z) of 285±0.5. In some embodiments, one or more ions comprise one or more fragment ions with a mass to charge ratio (m/z) of 154±0.5 or 193±0.5. In some embodiments, one or more ions comprise a prazepam precursor ion with a mass to charge ratio (m/z) of 325±0.5. In some embodiments, one or more ions comprise one or more fragment ions with a mass to charge ratio (m/z) of 165±0.5 or 271±0.5. In some embodiments, one or more ions comprise a medazepam precursor ion with a mass to charge ratio (m/z) of 271±0.5. In some embodiments, one or more ions comprise one or more fragment ions with a mass to charge ratio (m/z) of 180±0.5 or 207.1±0.5. In some embodiments, the analyte is an anti-epileptic drug. In some embodiments, the anti-epileptic drug is valproic acid, tiagabine, topiramate, levitiracetum, lamotrigine, lacosamide, ethosuximide, carbamazepine, eslicarbamazepine, 10,11-carbamazepine, phenobarbital, rufinamide, primidone, phenytoin, zonisamide, felbamate, gabapentin, or pregablin. In some embodiments, the anti-epileptic drug is selected from the group consisting of valproic acid, tiagabine, topiramate, levitiracetum, lamotrigine, lacosamide, ethosuximide, carbamazepine, eslicarbamazepine, 10,11-carbamazepine, phenobarbital, rufinamide, primidone, phenytoin, zonisamide, felbamate, gabapentin, and pregablin. In some embodiments, the anti-epileptic drug is extracted from a whole blood sample.

In some embodiments, one or more ions comprise a felbamate precursor ion with a mass to charge ratio (m/z) of 339±0.5. In some embodiments, one or more ions comprise one or more fragment ions with a mass to charge ratio (m/z) of 117.3±0.5 or 261±0.5. In some embodiments, one or more ions comprise a felbamate precursor ion with a mass to charge ratio (m/z) of 117±0.5. In some embodiments, one or more ions comprise one or more fragment ions with a mass to charge ratio (m/z) of 115±0.5 or 91±0.5. In some embodiments, one or more ions comprise an ethosuximide precursor ion with a mass to charge ratio (m/z) of 142±0.5. In some embodiments, one or more ions comprise one or more fragment ions with a mass to charge ratio (m/z) of 44.3±0.5 or 39.3±0.5. In some embodiments, one or more ions comprise a lacosamide precursor ion with a mass to charge ratio (m/z) of 251±0.5. In some embodiments, one or more ions comprise one or more fragment ions with a mass to charge ratio (m/z) of 91.2±0.5 or 65.2±0.5. In some embodiments, one or more ions comprise a lamotrigine precursor ion with a mass to charge ratio (m/z) of 256±0.5. In some embodiments, one or more ions comprise one or more fragment ions with a mass to charge ratio (m/z) of 211±0.5 or 145±0.5. In some embodiments, one or more ions comprise a topiramate precursor ion with a mass to charge ratio (m/z) of 338.2±0.5. In some embodiments, one or more ions comprise one or more fragment ions with a mass to charge ratio (m/z) of 78.2±0.5 or 96.2±0.5. In some embodiments, one or more ions comprise a gabapentin precursor ion with a mass to charge ratio (m/z) of 172.3±0.5. In some embodiments, one or more ions comprise one or more fragment ions with a mass to charge ratio (m/z) of 91.2±0.5 or 67.2±0.5. In some embodiments, one or more ions comprise an eslicarbazepine precursor ion with a mass to charge ratio (m/z) of 297.1±0.5. In some embodiments, one or more ions comprise one or more fragment ions with a mass to charge ratio (m/z) of 194±0.5 or 179±0.5. In some embodiments, one or more ions comprise a primidone precursor ion with a mass to charge ratio (m/z) of 219.8±0.5. In some embodiments, one or more ions comprise one or more fragment ions with a mass to charge ratio (m/z) of 79±0.5 or 135.2±0.5. In some embodiments, one or more ions comprise a pregabalin precursor ion with a mass to charge ratio (m/z) of 160.1±0.5. In some embodiments, one or more ions comprise one or more fragment ions with a mass to charge ratio (m/z) of 55.2±0.5 or 77.2±0.5. In some embodiments, one or more ions comprise a carbamazepine precursor ion with a mass to charge ratio (m/z) of 237±0.5. In some embodiments, one or more ions comprise one or more fragment ions with a mass to charge ratio (m/z) of 194.1±0.5 or 179±0.5. In some embodiments, one or more ions comprise a phenobarbital precursor ion with a mass to charge ratio (m/z) of 231±0.5. In some embodiments, one or more ions comprise one or more fragment ions with a mass to charge ratio (m/z) of 44.2±0.5 or 188.1±0.5. In some embodiments, one or more ions comprise an epoxide precursor ion with a mass to charge ratio (m/z) of 236.2±0.5. In some embodiments, one or more ions comprise one or more fragment ions with a mass to charge ratio (m/z) of 141.2±0.5 or 112.2±0.5. In some embodiments, one or more ions comprise a zonisamide precursor ion with a mass to charge ratio (m/z) of 213.2±0.5. In some embodiments, one or more ions comprise one or more fragment ions with a mass to charge ratio (m/z) of 77.2±0.5 or 102.1±0.5. In some embodiments, one or more ions comprise a tiagabine precursor ion with a mass to charge ratio (m/z) of 376.2±0.5. In some embodiments, one or more ions comprise one or more fragment ions with a mass to charge ratio (m/z) of 111.1±0.5 or 149.1±0.5. In some embodiments, one or more ions comprise a phenytoin precursor ion with a mass to charge ratio (m/z) of 253.1±0.5. In some embodiments, one or more ions comprise one or more fragment ions with a mass to charge ratio (m/z) of 104.2±0.5 or 182.2±0.5. In some embodiments, one or more ions comprise a levetiracetam precursor ion with a mass to charge ratio (m/z) of 171.2±0.5. In some embodiments, one or more ions comprise one or more fragment ions with a mass to charge ratio (m/z) of 126.2±0.5 or 69.2±0.5. In some embodiments, one or more ions comprise a valproic acid precursor ion with a mass to charge ratio (m/z) of 143±0.5. In some embodiments, one or more ions comprise one or more fragment ions with a mass to charge ratio (m/z) of 143±0.5. In some embodiments, one or more ions comprise a rufinamide precursor ion with a mass to charge ratio (m/z) of 239±0.5. In some embodiments, one or more ions comprise one or more fragment ions with a mass to charge ratio (m/z) of 127.2±0.5 or 261±0.5. In some embodiments, one or more ions comprise a primdone precursor ion with a mass to charge ratio (m/z) of 219±0.5. In some embodiments, one or more ions comprise one or more fragment ions with a mass to charge ratio (m/z) of 126±0.5 or 141±0.5. In some embodiments, one or more ions comprise a topiramate D12 precursor ion with a mass to charge ratio (m/z) of 350±0.5. In some embodiments, one or more ions comprise one or more fragment ions with a mass to charge ratio (m/z) of 78.2±0.5. In some embodiments, one or more ions comprise an epoxide D3 precursor ion with a mass to charge ratio (m/z) of 256±0.5. In some embodiments, one or more ions comprise one or more fragment ions with a mass to charge ratio (m/z) of 77±0.5. In some embodiments, one or more ions comprise a lamotrigine ¹³C₃ precursor ion with a mass to charge ratio (m/z) of 259±0.5. In some embodiments, one or more ions comprise one or more fragment ions with a mass to charge ratio (m/z) of 214±0.5. In some embodiments, one or more ions comprise a levetiracetam D6 precursor ion with a mass to charge ratio (m/z) of 177.2±0.5. In some embodiments, one or more ions comprise one or more fragment ions with a mass to charge ratio (m/z) of 132.2±0.5.

In some embodiments, the analyte is an immunosuppressant. In some embodiments, the immunosuppressant is cyclosporine A, sirolimus, tacrolimus, or everolimus. In some embodiments, the immunosuppressant is selected from the group consisting of cyclosporine A, sirolimus, tacrolimus, and everolimus. In some embodiments, the immunosuppressant is extracted from a whole blood sample.

In some embodiments, the analyte is a barbiturate. In some embodiments, the barbiturate is phenobarbitol, amobarbitol, butalbital, pentobarbitol, secobarbitol, or thiopental. In some embodiments, the barbiturate is selected from the group consisting of phenobarbitol, amobarbitol, butalbital, pentobarbitol, secobarbitol, and thiopental. In some embodiments, the barbiturate is extracted from a whole blood sample.

In some embodiments, one or more ions comprise a secobarbital precursor ion with a mass to charge ratio (m/z) of 237.0±0.5. In some embodiments, one or more ions comprise one or more fragment ions with a mass to charge ratio (m/z) of 42.0±0.5. In some embodiments, one or more ions comprise an ammobarbital precursor ion with a mass to charge ratio (m/z) of 225.0±0.5. In some embodiments, one or more ions comprise one or more fragment ions with a mass to charge ratio (m/z) of 182.0±0.5. In some embodiments, one or more ions comprise a pentobarbital precursor ion with a mass to charge ratio (m/z) of 225.6±0.5. In some embodiments, one or more ions comprise one or more fragment ions with a mass to charge ratio (m/z) of 42.0±0.5. In some embodiments, one or more ions comprise a thiopental precursor ion with a mass to charge ratio (m/z) of 241.0±0.5. In some embodiments, one or more ions comprise one or more fragment ions with a mass to charge ratio (m/z) of 57.9±0.5. In some embodiments, one or more ions comprise a phenobarbital precursor ion with a mass to charge ratio (m/z) of 231.0±0.5. In some embodiments, one or more ions comprise one or more fragment ions with a mass to charge ratio (m/z) of 42.0±0.5. In some embodiments, one or more ions comprise a butalbital precursor ion with a mass to charge ratio (m/z) of 223.1±0.5. In some embodiments, one or more ions comprise one or more fragment ions with a mass to charge ratio (m/z) of 42.1±0.5.

In some embodiments, the analyte is tamoxifen. In some embodiments, the analyte is a metabolite of tamoxifen. In some embodiments, said metabolite is norendoxifen. In some embodiments, said metabolite is endoxifen or N-Desmethyl-4-Hydroxy Tamoxifen. In some embodiments, said metabolite is 4′-Hydroxy Tamoxifen. In some embodiments, said metabolite is 4-Hydroxy Tamoxifen. In some embodiments, said metabolite is N-Desmethyl-4′-Hydroxy Tamoxifen. In some embodiments, said metabolite is N-Desmethyl Tamoxifen. In some embodiments, said metabolite is selected from the group consisting of norendoxifen, endoxifen, 4′-Hydroxy Tamoxifen, 4-Hydroxy Tamoxifen, N-Desmethyl-4′-Hydroxy Tamoxifen, and N-Desmethyl-4′-Hydroxy Tamoxifen. In some embodiments, tamoxifen or its metabolite is extracted from a whole blood sample.

In some embodiments, one or more ions comprise a tamoxifen precursor ion with a mass to charge ratio (m/z) of 372.2±0.5. In some embodiments, one or more ions comprise one or more fragment ions with a mass to charge ratio (m/z) of 72.14±0.5. In some embodiments, one or more ions comprise an endoxifen precursor ion with a mass to charge ratio (m/z) of 374.2±0.5. In some embodiments, one or more ions comprise one or more fragment ions with a mass to charge ratio (m/z) of 58.1±0.5. In some embodiments, one or more ions comprise a 4-hydroxy tamoxifen precursor ion with a mass to charge ratio (m/z) of 388.2±0.5. In some embodiments, one or more ions comprise one or more fragment ions with a mass to charge ratio (m/z) of 72.1±0.5. In some embodiments, one or more ions comprise an N-desmethyl-4′-hydroxy tamoxifen precursor ion with a mass to charge ratio (m/z) of 374.2±0.5. In some embodiments, one or more ions comprise one or more fragment ions with a mass to charge ratio (m/z) of 58.1±0.5. In some embodiments, one or more ions comprise a 4′-hydroxy tamoxifen precursor ion with a mass to charge ratio (m/z) of 388.2±0.5. In some embodiments, one or more ions comprise one or more fragment ions with a mass to charge ratio (m/z) of 72.1±0.5. In some embodiments, one or more ions comprise an N-desmethyl-4′-hydroxy tamoxifen precursor ion with a mass to charge ratio (m/z) of 358.2±0.5. In some embodiments, one or more ions comprise one or more fragment ions with a mass to charge ratio (m/z) of 58.1±0.5.

In some embodiments, the analyte is an oncology drug. In some embodiments, the analyte is anastrozole. In some embodiments, the analyte is letrozole. In some embodiments, the analyte is exemestane. In some embodiments, the analyte is selected from the group consisting of anastrozole, letrozole, and exemestane. In some embodiments, the oncology drug is extracted from a whole blood sample.

In some embodiments, the analyte is tetrahydrocannabinol (THC) or its metabolite. In some embodiments, THC is extracted from a urine sample.

In some embodiments, the extracted analyte is hydrolyzed. In some embodiments, the analyte is hydrolyzed prior to extraction.

In some embodiments, the collision energy is within the range of about 5 to 60 V. In some embodiments, the collision energy is within the range of about 5 to 60 V.

In another aspect, provided herein are methods for diagnosis of congenital adrenal hyperplasia in patients. In some embodiments, the methods of quantitation of endogenous steroids provided herein are used for diagnosing congenital adrenal hyperplasia.

In another aspect, provided herein are methods for detection or monitoring of THC use in an individual. In another aspect, provided herein are methods for detection or monitoring of barbiturate use in an individual. In another aspect, provided herein are methods for detection or monitoring of opiate use in an individual. In another aspect, provided herein are methods for detection or monitoring of benzodiazepine use in an individual.

In another aspect, provided herein are methods for detection or monitoring of anti-epileptic drug use in an individual. In another aspect, provided herein are methods for monitoring the anti-epileptic drug efficacy in an individual.

In another aspect, provided herein are methods for detection or monitoring of tamoxifen use in an individual. In another aspect, provided herein are methods for monitoring the tamoxifen efficacy in an individual.

In another aspect, certain methods presented herein utilize high resolution/high accuracy mass spectrometry to determine the amount of analyte in a sample. In some embodiments utilizing high accuracy/high resolution mass spectrometry, the methods include: (a) subjecting analyte from a sample to an ionization source under conditions suitable to generate ions, wherein the ions are detectable by mass spectrometry; and (b) determining the amount of one or more ions by high resolution/high accuracy mass spectrometry. In these embodiments, the amount of one or more ions determined in step (b) is related to the amount of analyte in the sample. In some embodiments, high resolution/high accuracy mass spectrometry is conducted at a FWHM of 10,000 and a mass accuracy of 50 ppm. In some embodiments, high resolution/high accuracy mass spectrometry is conducted with a high resolution/high accuracy time-of-flight (TOF) mass spectrometer. In some embodiments, the ionization conditions comprise ionization of analyte under acidic conditions. In some related embodiments, the acidic conditions comprise treatment of said sample with formic acid prior to ionization.

In any of the methods described herein, the sample may comprise a biological sample. In some embodiments, the biological sample may comprise a biological fluid such as urine, plasma, or serum. In some embodiments, the biological sample may comprise a sample from a human; such as from an adult male or female, or juvenile male or female, wherein the juvenile is under age 18, under age 15, under age 12, or under age 10. The human sample may be analyzed to diagnose or monitor a disease state or condition, or to monitor therapeutic efficacy of treatment of a disease state or condition. In some related embodiments, the methods described herein may be used to determine the amount of analyte in a biological sample when taken from a human.

In embodiments utilizing tandem mass spectrometry, tandem mass spectrometry may be conducted by any method known in the art, including for example, multiple reaction monitoring, precursor ion scanning, or product ion scanning.

In some embodiments, tandem mass spectrometry comprises fragmenting a precursor ion into one or more fragment ions. In embodiments where the amounts of two or more fragment ions are determined, the amounts may be subject to any mathematical manipulation known in the art in order to relate the measured ion amounts to the amount of analyte in the sample. For example, the amounts of two or more fragment ions may be summed as part of determining the amount of analyte in the sample.

In some embodiments, the high resolution/high accuracy mass spectrometry is conducted at a resolving power (FWHM) of greater than or equal to about 10,000, such as greater than or equal to about 15,000, such as greater than or equal to about 20,000, such as greater than or equal to about 25,000. In some embodiments, the high resolution/high accuracy mass spectrometry is conducted at an accuracy of less than or equal to about 50 ppm, such as less than or equal to about 20 ppm, such as less than or equal to about 10 ppm, such as less than or equal to about 5 ppm; such as less than or equal to about 3 ppm. In some embodiments, high resolution/high accuracy mass spectrometry is conducted at a resolving power (FWHM) of greater than or equal to about 10,000 and an accuracy of less than or equal to about 50 ppm. In some embodiments, the resolving power is greater than about 15,000 and the accuracy is less than or equal to about 20 ppm. In some embodiments, the resolving power is greater than or equal to about 20,000 and the accuracy is less than or equal to about 10 ppm; preferably resolving power is greater than or equal to about 20,000 and accuracy is less than or equal to about 5 ppm, such as less than or equal to about 3 ppm.

In some embodiments, the high resolution/high accuracy mass spectrometry may be conducted with an orbitrap mass spectrometer, a time of flight (TOF) mass spectrometer, or a Fourier transform ion cyclotron resonance mass spectrometer (sometimes known as a Fourier transform mass spectrometer).

Mass spectrometry (either tandem or high resolution/high accuracy) may be performed in positive ion mode. Alternatively, mass spectrometry may be performed in negative ion mode. Various ionization sources, including for example atmospheric pressure chemical ionization (APCI) or electrospray ionization (ESI), may be used to ionize the analyte.

In any method presented herein, a separately detectable internal standard may be provided in the sample, the amount of which is also determined in the sample. In embodiments utilizing a separately detectable internal standard, all or a portion of both the analyte of interest and the internal standard present in the sample is ionized to produce a plurality of ions detectable in a mass spectrometer, and one or more ions produced from each are detected by mass spectrometry. In these embodiments, the presence or amount of ions generated from the analyte of interest may be related to the presence of amount of analyte of interest in the sample by comparison to the amount of internal standard ions detected.

Alternatively, the amount of analyte in a sample may be determined by comparison to one or more external reference standards. Exemplary external reference standards include blank plasma or serum spiked with human or non-human analyte, a synthetic analyte analogue, or an isotopically labeled variant thereof.

The summary of the invention described above is non-limiting and other features and advantages of the invention will be apparent from the following detailed description of the invention, and from the claims.

### BRIEF DESCRIPTION OF THE DRAWINGS

FIG. 1 shows chromatogram of 14 steroids analyzed by mass spectrometry.

FIGS. 2-5 show normal levels of cortisol (FIG. 2), cortisone (FIG. 3), testosterone (FIG. 4), and androstenedione (FIG. 5) in a normal adult male, quantitated by the present assay.

FIGS. 6-10 show normal levels of progesterone (FIG. 6), cortisol (FIG. 7), cortisone (FIG. 8), androstenedione (FIG. 9), 17-OH progesterone (FIG. 10) in a normal adult female, quantitated by the present assay.

FIGS. 11-17 show levels of cortisol (FIG. 11), cortisone (FIG. 12), progesterone (FIG. 13), androstenedione (FIG. 14), testosterone (FIG. 15), 21-deoxycortisol (FIG. 16), and 17-OH progesterone (FIG. 17) in a child, quantitated by the present assay.

FIG. 18 shows standard linearity of testosterone between 50-10,000 ng/dL.

FIG. 19 shows chromatogram of tamoxifen and its metabolites.

FIG. 20 shows chromatogram of letrozole, exemestane, and anastrozole.

FIG. 21 shows exemplary chromatograms of opiates (oxymorphone, hydromorphone, and codeine) and corresponding internal standards.

FIG. 22 shows exemplary chromatograms of opiates (noroxycodone, oxycodone, and norhydrocodone) and corresponding internal standards.

FIG. 23 shows exemplary chromatograms of opiates (morphine, hydrocodone, and norfentanyl) and corresponding internal standards.

FIG. 24 shows exemplary chromatogram of opiate (fentanyl) and corresponding internal standard.

FIGS. 25 to 28 show morphine, codeine, hydromorphone, and oxycodone (respectively) data obtained from patient urine using 20 uL MITRA® tip with glucuronidase hydrolysis.

FIG. 29 shows oxycodone data obtained from patient saliva using 50 uL MITRA® tip.

FIGS. 30 and 31 show the results of hematocrit study of buprenorphine and norfentanyl, respectively.

FIGS. 32 and 33 show the results of negative urine spiked with barbiturates (secobarbital, ammobarbital, pentobarbital, and thiopental).

FIGS. 34 to 38 show the results of various patient samples quantitated for phenobarbital and butalbital.

FIG. 39 shows the results of THC carboxy metabolite analysis in patient sample using 20 uL tip and glucuronidase hydrolysis.

FIG. 40 shows the results of hematocrit study of gabapentin and rufinamide.

FIG. 41 shows the chromatogram of the 25-hydroxyvitamin D analysis.

FIG. 42 shows the calibration curve of 25-hydroxyvitamin D2 analysis.

FIG. 43 shows the calibration curve of 25-hydroxyvitamin D3 analysis.

### DETAILED DESCRIPTION OF THE INVENTION

As used herein, unless otherwise stated, the singular forms “a,” “an,” and “the” include plural reference. Thus, for example, a reference to “a protein” includes a plurality of protein molecules.

As used herein, the terms “purification”, “purifying”, and “enriching” do not refer to removing all materials from the sample other than the analyte(s) of interest. Instead, these terms refer to a procedure that enriches the amount of one or more analytes of interest relative to other components in the sample that may interfere with detection of the analyte of interest. Purification of the sample by various means may allow relative reduction of one or more interfering substances, e.g., one or more substances that may or may not interfere with the detection of selected parent or daughter ions by mass spectrometry. Relative reduction as this term is used does not require that any substance, present with the analyte of interest in the material to be purified, is entirely removed by purification.

As used herein, the term “immunopurification” or “immunopurify” refers to a purification procedure that utilizes antibodies, including polyclonal or monoclonal antibodies, to enrich the one or more analytes of interest. Immunopurification can be performed using any of the immunopurification methods well known in the art. Often the immunopurification procedure utilizes antibodies bound, conjugated or otherwise attached to a solid support, for example a column, well, tube, gel, capsule, particle or the like. Immunopurification as used herein includes without limitation procedures often referred to in the art as immunoprecipitation, as well as procedures often referred to in the art as affinity chromatography or immunoaffinity chromatography.

As used herein, the term “immunoparticle” refers to a capsule, bead, gel particle or the like that has antibodies bound, conjugated or otherwise attached to its surface (either on and/or in the particle). In certain preferred embodiments, immunoparticles are sepharose or agarose beads. In alternative preferred embodiments, immunoparticles comprise glass, plastic or silica beads, or silica gel.

As used herein, the term “sample” refers to any sample that may contain an analyte of interest. As used herein, the term “body fluid” means any fluid that can be isolated from the body of an individual. For example, “body fluid” may include blood, plasma, serum, bile, saliva, urine, tears, perspiration, and the like. In preferred embodiments, the sample comprises a body fluid sample from human; preferably plasma or serum.

As used herein, the term “solid phase extraction” or “SPE” refers to a process in which a chemical mixture is separated into components as a result of the affinity of components dissolved or suspended in a solution (i.e., mobile phase) for a solid through or around which the solution is passed (i.e., solid phase). In some instances, as the mobile phase passes through or around the solid phase, undesired components of the mobile phase may be retained by the solid phase resulting in a purification of the analyte in the mobile phase. In other instances, the analyte may be retained by the solid phase, allowing undesired components of the mobile phase to pass through or around the solid phase. In these instances, a second mobile phase is then used to elute the retained analyte off of the solid phase for further processing or analysis. SPE, including TFLC, may operate via a unitary or mixed mode mechanism. Mixed mode mechanisms utilize ion exchange and hydrophobic retention in the same column; for example, the solid phase of a mixed-mode SPE column may exhibit strong anion exchange and hydrophobic retention; or may exhibit strong cation exchange and hydrophobic retention.

Generally, the affinity of a SPE column packing material for an analyte may be due to any of a variety of mechanisms, such as one or more chemical interactions or an immunoaffinity interaction. In some embodiments, SPE of analyte is conducted without the use of an immunoaffinity column packing material. That is, in some embodiments, analyte is purified from a sample by a SPE column that is not an immunoaffinity column.

As used herein, the term “chromatography” refers to a process in which a chemical mixture carried by a liquid or gas is separated into components as a result of differential distribution of the chemical entities as they flow around or over a stationary liquid or solid phase.

As used herein, the term “liquid chromatography” or “LC” means a process of selective retardation of one or more components of a fluid solution as the fluid uniformly percolates through a column of a finely divided substance, or through capillary passageways. The retardation results from the distribution of the components of the mixture between one or more stationary phases and the bulk fluid, (i.e., mobile phase), as this fluid moves relative to the stationary phase(s). Examples of “liquid chromatography” include reverse phase liquid chromatography (RPLC), high performance liquid chromatography (HPLC), and turbulent flow liquid chromatography (TFLC) (sometimes known as high turbulence liquid chromatography (HTLC) or high throughput liquid chromatography).

As used herein, the term “high performance liquid chromatography” or “HPLC” (sometimes known as “high pressure liquid chromatography”) refers to liquid chromatography in which the degree of separation is increased by forcing the mobile phase under pressure through a stationary phase, typically a densely packed column.

As used herein, the term “turbulent flow liquid chromatography” or “TFLC” (sometimes known as high turbulence liquid chromatography or high throughput liquid chromatography) refers to a form of chromatography that utilizes turbulent flow of the material being assayed through the column packing as the basis for performing the separation. TFLC has been applied in the preparation of samples containing two unnamed drugs prior to analysis by mass spectrometry. See, e.g., Zimmer et al., J Chromatogr A 854: 23-35 (1999); see also, U.S. Pat. Nos. 5,968,367, 5,919,368, 5,795,469, and 5,772,874, which further explain TFLC. Persons of ordinary skill in the art understand “turbulent flow”. When fluid flows slowly and smoothly, the flow is called “laminar flow”. For example, fluid moving through an HPLC column at low flow rates is laminar. In laminar flow the motion of the particles of fluid is orderly with particles moving generally in substantially straight lines. At faster velocities, the inertia of the water overcomes fluid frictional forces and turbulent flow results. Fluid not in contact with the irregular boundary “outruns” that which is slowed by friction or deflected by an uneven surface. When a fluid is flowing turbulently, it flows in eddies and whirls (or vortices), with more “drag” than when the flow is laminar. Many references are available for assisting in determining when fluid flow is laminar or turbulent (e.g., Turbulent Flow Analysis: Measurement and Prediction, P. S. Bernard & J. M. Wallace, John Wiley & Sons, Inc., (2000); An Introduction to Turbulent Flow, Jean Mathieu & Julian Scott, Cambridge University Press (2001)).

As used herein, the term “gas chromatography” or “GC” refers to chromatography in which the sample mixture is vaporized and injected into a stream of carrier gas (as nitrogen or helium) moving through a column containing a stationary phase composed of a liquid or a particulate solid and is separated into its component compounds according to the affinity of the compounds for the stationary phase.

As used herein, the term “large particle column” or “extraction column” refers to a chromatography column containing an average particle diameter greater than about 50 μm. As used in this context, the term “about” means±10%.

As used herein, the term “analytical column” refers to a chromatography column having sufficient chromatographic plates to effect a separation of materials in a sample that elute from the column sufficient to allow a determination of the presence or amount of an analyte. Such columns are often distinguished from “extraction columns”, which have the general purpose of separating or extracting retained material from non-retained materials in order to obtain a purified sample for further analysis. As used in this context, the term “about” means±10%. In a preferred embodiment the analytical column contains particles of about 5 μm in diameter.

As used herein, the terms “on-line” and “inline”, for example as used in “on-line automated fashion” or “on-line extraction”, refers to a procedure performed without the need for operator intervention. In contrast, the term “off-line” as used herein refers to a procedure requiring manual intervention of an operator. Thus, if samples are subjected to precipitation and the supernatants are then manually loaded into an autosampler, the precipitation and loading steps are off-line from the subsequent steps. In various embodiments of the methods, one or more steps may be performed in an on-line automated fashion.

As used herein, the term “mass spectrometry” or “MS” refers to an analytical technique to identify compounds by their mass. MS refers to methods of filtering, detecting, and measuring ions based on their mass-to-charge ratio, or “m/z”. MS technology generally includes (1) ionizing the compounds to form charged compounds; and (2) detecting the molecular weight of the charged compounds and calculating a mass-to-charge ratio. The compounds may be ionized and detected by any suitable means. A “mass spectrometer” generally includes an ionizer, a mass analyzer, and an ion detector. In general, one or more molecules of interest are ionized, and the ions are subsequently introduced into a mass spectrometric instrument where, due to a combination of magnetic and electric fields, the ions follow a path in space that is dependent upon mass (“m”) and charge (“z”). See, e.g., U.S. Pat. No. 6,204,500, entitled “Mass Spectrometry From Surfaces;” U.S. Pat. No. 6,107,623, entitled “Methods and Apparatus for Tandem Mass Spectrometry;” U.S. Pat. No. 6,268,144, entitled “DNA Diagnostics Based On Mass Spectrometry;” U.S. Pat. No. 6,124,137, entitled “Surface-Enhanced Photolabile Attachment And Release For Desorption And Detection Of Analytes;” Wright et al., Prostate Cancer and Prostatic Diseases 1999, 2: 264-76; and Merchant and Weinberger, Electrophoresis 2000, 21: 1164-67.

As used herein, “high resolution/high accuracy mass spectrometry” refers to mass spectrometry conducted with a mass analyzer capable of measuring the mass to charge ratio of a charged species with sufficient precision and accuracy to confirm a unique chemical ion. Confirmation of a unique chemical ion is possible for an ion when individual isotopic peaks from that ion are readily discernable. The particular resolving power and mass accuracy necessary to confirm a unique chemical ion varies with the mass and charge state of the ion.

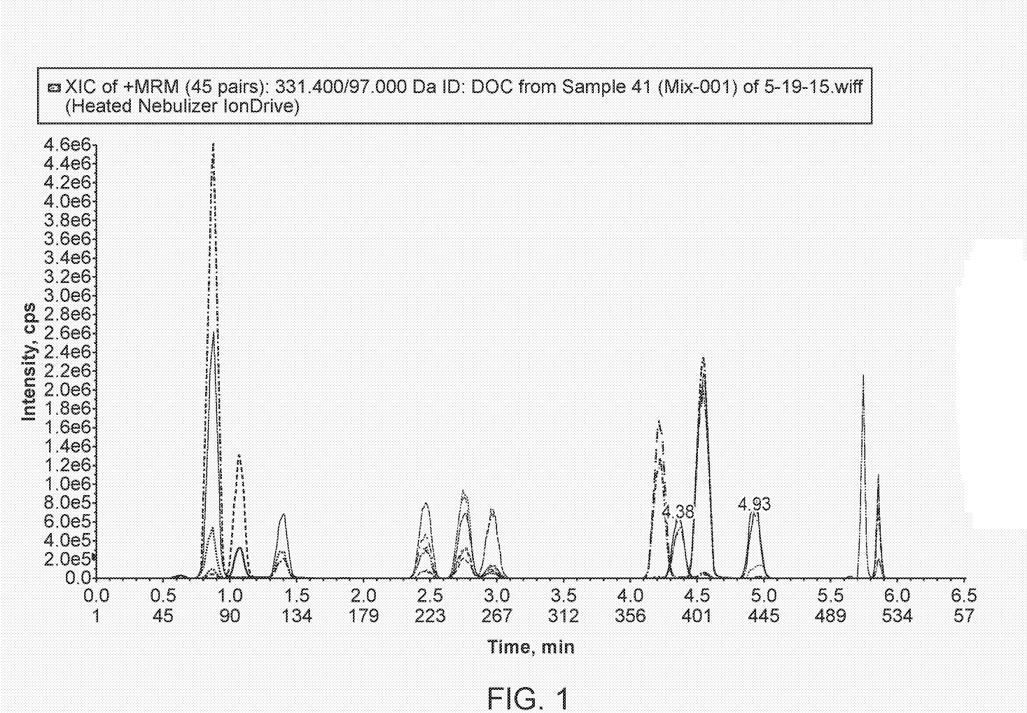

As used herein, the term “resolving power” or “resolving power (FWHM)” (also known in the art as “m/Δm_{50%}”) refers to an observed mass to charge ratio divided by the width of the mass peak at 50% maximum height (Full Width Half Maximum, “FWHM”). The effect of differences in resolving power is illustrated in FIGS. 1A-C, which show theoretical mass spectra of an ion with a m/z of about 1093. FIG. 1A shows a theoretical mass spectrum from a mass analyzer with resolving power of about 3000 (a typical operating condition for a conventional quadrupole mass analyzer). As seen in FIG. 1A, no individual isotopic peaks are discernable. By comparison, FIG. 1B shows a theoretical mass spectrum from a mass analyzer with resolving power of about 10,000, with clearly discernable individual isotopic peaks. FIG. 1C shows a theoretical mass spectrum from a mass analyzer with resolving power of about 12,000. At this highest resolving power, the individual isotopic peaks contain less than 1% contribution from baseline.

As used herein a “unique chemical ion” with respect to mass spectrometry refers a single ion with a single atomic makeup. The single ion may be singly or multiply charged.

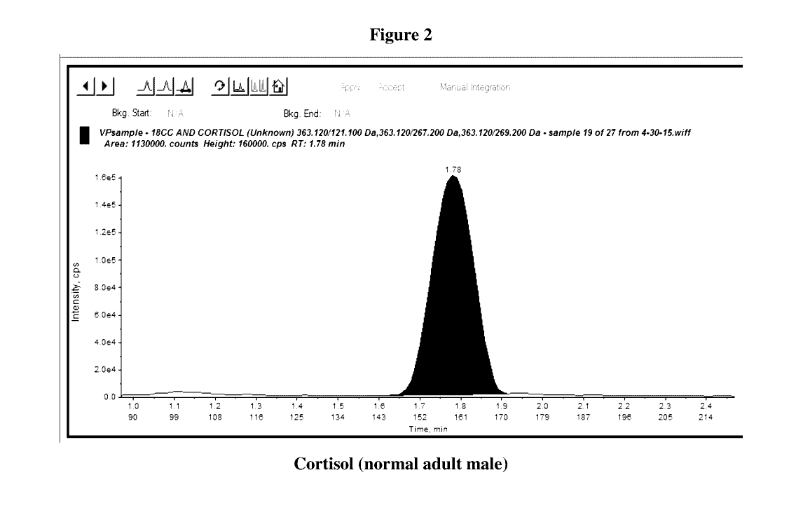

As used herein, the term “accuracy” (or “mass accuracy”) with respect to mass spectrometry refers to potential deviation of the instrument response from the true m/z of the ion investigated. Accuracy is typically expressed in parts per million (ppm). The effect of differences in mass accuracy is illustrated in FIGS. 2A-D, which show the boundaries of potential differences between a detected m/z and the actual m/z for a theoretical peak at m/z of 1093.52094. FIG. 2A shows the potential range of detected m/z at an accuracy of 120 ppm. By contrast, FIG. 2B shows the potential range of detected m/z at an accuracy of 50 ppm. FIGS. 2C and 2D show the even narrower potential ranges of detected m/z at accuracies of 20 ppm and 10 ppm.

High resolution/high accuracy mass spectrometry methods of the present invention may be conducted on instruments capable of performing mass analysis with FWHM of greater than 10,000, 15,000, 20,000, 25,000, 50,000, 100,000, or even more. Likewise, methods of the present invention may be conducted on instruments capable of performing mass analysis with accuracy of less than 50 ppm, 20 ppm, 15 ppm, 10 ppm, 5 ppm, 3 ppm, or even less. Instruments capable of these performance characteristics may incorporate certain orbitrap mass analyzers, time-of-flight (“TOF”) mass analyzers, or Fourier-transform ion cyclotron resonance mass analyzers. In preferred embodiments, the methods are carried out with an instrument which includes an orbitrap mass analyzer or a TOF mass analyzer.

The term “orbitrap” describes an ion trap consisting of an outer barrel-like electrode and a coaxial inner electrode. Ions are injected tangentially into the electric field between the electrodes and trapped because electrostatic interactions between the ions and electrodes are balanced by centrifugal forces as the ions orbit the coaxial inner electrode. As an ion orbits the coaxial inner electrode, the orbital path of a trapped ion oscillates along the axis of the central electrode at a harmonic frequency relative to the mass to charge ratio of the ion. Detection of the orbital oscillation frequency allows the orbitrap to be used as a mass analyzer with high accuracy (as low as 1-2 ppm) and high resolving power (FWHM) (up to about 200,000). A mass analyzer based on an orbitrap is described in detail in U.S. Pat. No. 6,995,364, incorporated by reference herein in its entirety. Use of orbitrap analyzers has been reported for qualitative and quantitative analyses of various analytes. See, e.g., U.S. Patent Application Pub. No. 2008/0118932 (filed Nov. 9, 2007); Bredehoft, et al., Rapid Commun. Mass Spectrom., 2008, 22:477-485; Le Breton, et al., Rapid Commun. Mass Spectrom., 2008, 22:3130-36; Thevis, et al., Mass Spectrom. Reviews, 2008, 27:35-50; Thomas, et al., J. Mass Spectrom., 2008, 43:908-15; Schenk, et al., BMC Medical Genomics, 2008, 1:41; and Olsen, et al., Nature Methods, 2007, 4:709-12.

As used herein, the term “operating in negative ion mode” refers to those mass spectrometry methods where negative ions are generated and detected. The term “operating in positive ion mode” as used herein, refers to those mass spectrometry methods where positive ions are generated and detected. In preferred embodiments, mass spectrometry is conducted in positive ion mode.

As used herein, the term “ionization” or “ionizing” refers to the process of generating an analyte ion having a net electrical charge equal to one or more electron units. Negative ions are those having a net negative charge of one or more electron units, while positive ions are those having a net positive charge of one or more electron units.

As used herein, the term “electron ionization” or “EI” refers to methods in which an analyte of interest in a gaseous or vapor phase interacts with a flow of electrons. Impact of the electrons with the analyte produces analyte ions, which may then be subjected to a mass spectrometry technique.

As used herein, the term “chemical ionization” or “CI” refers to methods in which a reagent gas (e.g. ammonia) is subjected to electron impact, and analyte ions are formed by the interaction of reagent gas ions and analyte molecules.

As used herein, the term “fast atom bombardment” or “FAB” refers to methods in which a beam of high energy atoms (often Xe or Ar) impacts a non-volatile sample, desorbing and ionizing molecules contained in the sample. Test samples are dissolved in a viscous liquid matrix such as glycerol, thioglycerol, m-nitrobenzyl alcohol, 18-crown-6 crown ether, 2-nitrophenyloctyl ether, sulfolane, diethanolamine, and triethanolamine. The choice of an appropriate matrix for a compound or sample is an empirical process.

As used herein, the term “matrix-assisted laser desorption ionization” or “MALDI” refers to methods in which a non-volatile sample is exposed to laser irradiation, which desorbs and ionizes analytes in the sample by various ionization pathways, including photo-ionization, protonation, deprotonation, and cluster decay. For MALDI, the sample is mixed with an energy-absorbing matrix, which facilitates desorption of analyte molecules.

As used herein, the term “surface enhanced laser desorption ionization” or “SELDI” refers to another method in which a non-volatile sample is exposed to laser irradiation, which desorbs and ionizes analytes in the sample by various ionization pathways, including photo-ionization, protonation, deprotonation, and cluster decay. For SELDI, the sample is typically bound to a surface that preferentially retains one or more analytes of interest. As in MALDI, this process may also employ an energy-absorbing material to facilitate ionization.

As used herein, the term “electrospray ionization” or “ESI,” refers to methods in which a solution is passed along a short length of capillary tube, to the end of which is applied a high positive or negative electric potential. Solution reaching the end of the tube is vaporized (nebulized) into a jet or spray of very small droplets of solution in solvent vapor. This mist of droplets flows through an evaporation chamber. As the droplets get smaller the electrical surface charge density increases until such time that the natural repulsion between like charges causes ions as well as neutral molecules to be released.

As used herein, the term “atmospheric pressure chemical ionization” or “APCI,” refers to mass spectrometry methods that are similar to ESI; however, APCI produces ions by ion-molecule reactions that occur within a plasma at atmospheric pressure. The plasma is maintained by an electric discharge between the spray capillary and a counter electrode. Then ions are typically extracted into the mass analyzer by use of a set of differentially pumped skimmer stages. A counterflow of dry and preheated N₂ gas may be used to improve removal of solvent. The gas-phase ionization in APCI can be more effective than ESI for analyzing less-polar species.

The term “atmospheric pressure photoionization” or “APPI” as used herein refers to the form of mass spectrometry where the mechanism for the ionization of molecule M is photon absorption and electron ejection to form the molecular ion M+. Because the photon energy typically is just above the ionization potential, the molecular ion is less susceptible to dissociation. In many cases it may be possible to analyze samples without the need for chromatography, thus saving significant time and expense. In the presence of water vapor or protic solvents, the molecular ion can extract H to form MH+. This tends to occur if M has a high proton affinity. This does not affect quantitation accuracy because the sum of M+ and MH+ is constant. Drug compounds in protic solvents are usually observed as MH+, whereas nonpolar compounds such as naphthalene or testosterone usually form M+. See, e.g., Robb et al., Anal. Chem. 2000, 72(15): 3653-3659.

As used herein, the term “inductively coupled plasma” or “ICP” refers to methods in which a sample interacts with a partially ionized gas at a sufficiently high temperature such that most elements are atomized and ionized.

As used herein, the term “field desorption” refers to methods in which a non-volatile test sample is placed on an ionization surface, and an intense electric field is used to generate analyte ions.

As used herein, the term “desorption” refers to the removal of an analyte from a surface and/or the entry of an analyte into a gaseous phase. Laser desorption thermal desorption is a technique wherein a sample containing the analyte is thermally desorbed into the gas phase by a laser pulse. The laser hits the back of a specially made 96-well plate with a metal base. The laser pulse heats the base and the heat causes the sample to transfer into the gas phase. The gas phase sample is then drawn into the mass spectrometer.

As used herein, the term “selective ion monitoring” is a detection mode for a mass spectrometric instrument in which only ions within a relatively narrow mass range, typically about one mass unit, are detected.

As used herein, “multiple reaction mode,” sometimes known as “selected reaction monitoring,” is a detection mode for a mass spectrometric instrument in which a precursor ion and one or more fragment ions are selectively detected.

As used herein, the term “lower limit of quantification”, “lower limit of quantitation” or “LLOQ” refers to the point where measurements become quantitatively meaningful. The analyte response at this LOQ is identifiable, discrete and reproducible with a relative standard deviation (RSD %) of less than 20% and an accuracy of 85% to 115%.

As used herein, the term “limit of detection” or “LOD” is the point at which the measured value is larger than the uncertainty associated with it. The LOD is the point at which a value is beyond the uncertainty associated with its measurement and is defined as three times the RSD of the mean at the zero concentration.

As used herein, an “amount” of an analyte in a body fluid sample refers generally to an absolute value reflecting the mass of the analyte detectable in volume of sample. However, an amount also contemplates a relative amount in comparison to another analyte amount. For example, an amount of an analyte in a sample can be an amount which is greater than a control or normal level of the analyte normally present in the sample.

The term “about” as used herein in reference to quantitative measurements not including the measurement of the mass of an ion, refers to the indicated value plus or minus 10%. Mass spectrometry instruments can vary slightly in determining the mass of a given analyte. The term “about” in the context of the mass of an ion or the mass/charge ratio of an ion refers to +/−0.50 atomic mass unit.

Collection of venous blood from newborn can be problematic. Although the minimum serum volume needed for the comprehensive steroid panel (or CAH panel) is minimal, at least 1-2 mL of whole blood is acquired through venipuncture. Using a microsampling device (Mitra tip) requires only 20 uL of capillary blood and makes it easier and less invasive, especially for neonates, which eliminates the need to do venipuncture.

In one aspect, provided herein are methods for mass spectrometric quantitation of analytes collected and extracted from a microsampling device.

In certain embodiments, the methods provided herein are directed to quantitating the amount of an analyte in a sample comprising (a) extracting an analyte from a sample collected by a microsampling device; (b) ionizing the analyte to generate one or more ions detectable by mass spectrometry; and (c) determining the amount of the one or more ions by mass spectrometry. In some embodiments, the amount of the one or more ions determined is used to determine the amount of analyte in the sample. In some embodiments, the amount of analyte in the sample is related to the amount of analyte in the patient.

In some embodiments, the methods provided herein comprise purifying the samples prior to mass spectrometry. In some embodiments, the methods comprise purifying the samples using liquid chromatography. In some embodiments, liquid chromatography comprise high performance liquid chromatography (HPLC) or high turbulence liquid chromatograph (HTLC). In some embodiments, the methods comprise subjecting a sample to solid phase extraction (SPE). In some embodiments, the methods comprise subjecting a sample to reverse phase analytical column.

In some embodiments, the methods provided herein are directed to quantitating the amount of an analyte in a sample comprising (a) extracting an analyte from a sample collected by a microsampling device, (b) purifying the sample by liquid chromatography, (c) ionizing the analyte to generate one or more ions detectable by mass spectrometry; and (d) determining the amount of the one or more ions by mass spectrometry. In some embodiments, the amount of the one or more ions determined is used to determine the amount of analyte in the sample. In some embodiments, the amount of analyte in the sample is related to the amount of analyte in the patient.

In some embodiments, mass spectrometry comprises tandem mass spectrometry. In some embodiments, mass spectrometry is high resolution mass spectrometry. In some embodiments, mass spectrometry is high resolution/high accuracy mass spectrometry.

In some embodiments, ionization is by atmospheric pressure chemical ionization (APCI). In some embodiments, ionization is by electrospray ionization (ESI). In some embodiments, said ionization is in positive ion mode. In some embodiments, said ionization is in negative ion mode.

In some embodiments, the microsampling device containing the sample is placed in a 96-well plate. In some embodiments, the microsampling device containing the sample is placed in a 96-rack. In some embodiments, automation places the 96-rack into a 96-well plate. In some embodiments, the automation is HAMILTON® automation.

In some embodiments, the methods provided herein comprise adding internal standards to the sample. In some embodiments, the internal standard is labeled. In some embodiments, the internal standard is deuterated or isotopically labeled. In some embodiments, the internal standard is added with extraction buffer. In some embodiments, the microsampling device is pre-soaked with internal standard and dried.

In some embodiments, the extracting step comprises adding an extraction buffer to the sample collected by a microsampling device. In some embodiments, the extracting step comprises placing the microsampling device containing the sample into a 96-well plate containing an extraction solvent. In some embodiments, the extraction step is automated. In some embodiments, 96-well plate is vortexed and then the absorbent tips of the microsampling device are removed. In some embodiments, the extracting step comprises drying down under nitrogen. In some embodiments, the extracting step comprises reconstituting the sample into solution. In some embodiments, the reconstitution comprises adding aqueous acid or organic solution or both to the sample. In some embodiments, the reconstituted solution is filtered.

In some embodiments, the extracted sample is injected into a mass spectrometric system. In some embodiments, the extracted sample is injected into liquid chromatography. In some embodiments, the extraction and mass spectrometry steps are performed in an on-line fashion to allow for automated sample analysis. In some embodiments, the extraction, purification, and mass spectrometry steps are performed in an on-line fashion to allow for automated sample analysis.

In some embodiments, the analyte is underivatized.

In some embodiments, the sample collected by the microsampling device does not require sample processing.

In some embodiments, the sample collected by the microsampling device is whole blood. In some embodiments, the sample collected by the microsampling device is urine. In some embodiments, the sample collected by the microsampling device is saliva. In some embodiments, the sample collected by the microsampling device is serum or plasma.

In some embodiments, the microsampling device comprises an absorbent tip that collects the sample. In some embodiments, the sample collected by the microsampling device absorbs a fixed volume of patient fluids. In some embodiments, the sample collected by the microsampling device has a volume of less than or equal to 150 μL. In some embodiments, the sample collected by the microsampling device has a volume of less than or equal to 100 μL. In some embodiments, the sample collected by the microsampling device has a volume of less than or equal to 50 μL. In some embodiments, the sample collected by the microsampling device has a volume of between 5 μL and 150 μL, inclusive. In some embodiments, the sample collected by the microsampling device has a volume of between 10 μL and 100 μL, inclusive. In some embodiments, the sample collected by the microsampling device has a volume of about 10 μL. In some embodiments, the sample collected by the microsampling device has a volume of about 15 μL. In some embodiments, the sample collected by the microsampling device has a volume of about 20 μL. In some embodiments, the sample collected by the microsampling device has a volume of about 30 μL. In some embodiments, the sample collected by the microsampling device has a volume of about 50 μL. In some embodiments, the sample collected by the microsampling device has a volume of about 100 μL. In some embodiments, the sample collected by the microsampling device absorbs a fixed volume of blood, regardless of the amount of hematocrit.

In some embodiments, the methods provided herein are directed to quantitating the amount of an analyte in a sample comprising (a) extracting an analyte from a sample of less than or equal to 100 μL; (b) ionizing the analyte to generate one or more ions detectable by mass spectrometry; and (c) determining the amount of the one or more ions by mass spectrometry. In some embodiments, the amount of the one or more ions determined is used to determine the amount of analyte in the sample. In some embodiments, the amount of analyte in the sample is related to the amount of analyte in the patient.

In some embodiments, the methods provided herein are directed to quantitating the amount of an analyte in a sample comprising (a) extracting an analyte from a sample of less than or equal to 100 μL; (b) purifying the sample by liquid chromatography; (c) ionizing the analyte to generate one or more ions detectable by mass spectrometry; and (d) determining the amount of the one or more ions by mass spectrometry. In some embodiments, the amount of the one or more ions determined is used to determine the amount of analyte in the sample. In some embodiments, the amount of analyte in the sample is related to the amount of analyte in the patient.

In some embodiments, the methods comprise extracting an analyte from a sample of less than or equal to 50 μL. In some embodiments, the methods comprise extracting an analyte from a sample of less than or equal to 30 μL. In some embodiments, the methods comprise extracting an analyte from a sample of less than or equal to 20 μL. In some embodiments, the methods comprise extracting an analyte from a sample of less than or equal to 15 μL. In some embodiments, the methods comprise extracting an analyte from a sample of less than or equal to 10 μL.

In some embodiments, the sample collected by the microsampling device can be transported without refrigeration or freezing. In some embodiments, the sample collected by the microsampling device can be transported without dry ice. In some embodiments, the sample collected by the microsampling device can be transported at room temperature. In some embodiments, the sample collected by the microsampling device can be transported without biohazard concerns.

In some embodiments, the sample collected by the microsampling device requires little training for collection. In some embodiments, the sample collected by the microsampling device can be collected anywhere. In some embodiments, the sample collected by the microsampling device can be dried at ambient temperature for shipping.

In some embodiments, the microsampling device is a MITRA® tip. In some embodiments, the microsampling device is encased in a cartridge designed for automation of extraction and mass spectrometric analysis.

In some embodiments, the methods further comprise collecting the sample with a microsampling device. In some embodiments, the collecting step comprises performing a finger prick and applying an absorbent tip of the microsampling device to the blood. In some embodiments, the collecting step comprises applying an absorbent tip in the urine or saliva of the patient. In some embodiments, the sample collected in the microsampling device is air dried. In some embodiments, the sample collected in the microsampling device is air dried for 1 to 2 hours prior to transport.

In some embodiments, the analyte is a steroid. In some embodiments, the steroid is cortisol, cortisone, progesterone, 17-hydroxyprogesterone, androstenedione, testosterone, dehydroepiandrosterone, corticosterone, deoxycorticosterone, 11-deoxycortisol, pregnenolone, 17-hydroxypregnenolone, 18-hydroxycorticosterone, or 21-deoxycortisol. In some embodiments, the analyte is a steroid in a steroid panel for diagnosing congenital adrenal hyperplasia (CAH). In some embodiments, the steroid is selected from the group consisting of cortisol, cortisone, progesterone, 17-hydroxyprogesterone, androstenedione, testosterone, dehydroepiandrosterone, corticosterone, deoxycorticosterone, 11-deoxycortisol, pregnenolone, 17-hydroxypregnenolone, 18-hydroxycorticosterone, and 21-deoxycortisol. In some embodiments, the steroid is 25-hydroxyvitamin D₂ or 25-hydroxyvitamin D₃.

In some embodiments, the analyte is an opiate. In some embodiments, the opiate is cis-tramadol, O-desmethyl tramadol, tapentadol, N-desmethyltapentadol, codeine, morphine, oxymorphone, norhydrocodone, oxycodone, noroxycodone, hydromorphone, hydrocodone, buprenorphine, norbuprenorphine, fentanyl, norfentanyl, 6-monoacetylmorphine (6-MAM), methadone, dihydrocodeine, naloxone, naltrexone, 6β-naltrexol, nalorphine, nalbuphine, or 2-ethylidene-1,5-dimethyl-3,3-diphenylpyrrolidine (EDDP). In some embodiments, the opiate is selected from the group consisting of cis-tramadol, O-desmethyl tramadol, tapentadol, N-desmethyltapentadol, codeine, morphine, oxymorphone, norhydrocodone, oxycodone, noroxycodone, hydromorphone, hydrocodone, buprenorphine, norbuprenorphine, fentanyl, norfentanyl, 6-monoacetylmorphine (6-MAM), methadone, dihydrocodeine, naloxone, naltrexone, 6β-naltrexol, nalorphine, nalbuphine, and 2-ethylidene-1,5-dimethyl-3,3-diphenylpyrrolidine (EDDP). In some embodiments, the opiate is extracted from a whole blood, salive, or urine sample.

In some embodiments, the analyte is a benzodiazepine. In some embodiments, the benzodiazepine is oxazepam, temazepam, lorazepam, nordiazepam, diazepam, chlordiazepoxide, triazolam, midazolam, alprazolam, clonazepam, bromazepam, clobazam, nitrazepam, phenazepam, prazepam, medazepam, flunitrazepam, or flurazepam. In some embodiments, the benzodiazepine is selected from the group consisting of oxazepam, temazepam, lorazepam, nordiazepam, diazepam, chlordiazepoxide, triazolam, midazolam, alprazolam, clonazepam, bromazepam, clobazam, nitrazepam, phenazepam, prazepam, medazepam, flunitrazepam, and flurazepam. In some embodiments, the benzodiazepine is extracted from a whole blood or urine sample.

In some embodiments, one or more ions comprise a bromazepam precursor ion with a mass to charge ratio (m/z) of 316±0.5. In some embodiments, one or more ions comprise one or more fragment ions with a mass to charge ratio (m/z) of 214±0.5 or 270±0.5. In some embodiments, one or more ions comprise an oxazepam precursor ion with a mass to charge ratio (m/z) of 287±0.5. In some embodiments, one or more ions comprise one or more fragment ions with a mass to charge ratio (m/z) of 104±0.5 or 241±0.5. In some embodiments, one or more ions comprise an clobazam precursor ion with a mass to charge ratio (m/z) of 300±0.5. In some embodiments, one or more ions comprise one or more fragment ions with a mass to charge ratio (m/z) of 224±0.5 or 259±0.5. In some embodiments, one or more ions comprise a nitrazepam precursor ion with a mass to charge ratio (m/z) of 282±0.5. In some embodiments, one or more ions comprise one or more fragment ions with a mass to charge ratio (m/z) of 180±0.5 or 236±0.5. In some embodiments, one or more ions comprise an alprazolam precursor ion with a mass to charge ratio (m/z) of 309.1±0.5. In some embodiments, one or more ions comprise one or more fragment ions with a mass to charge ratio (m/z) of 165±0.5 or 280.9±0.5. In some embodiments, one or more ions comprise an triazolam precursor ion with a mass to charge ratio (m/z) of 343±0.5. In some embodiments, one or more ions comprise one or more fragment ions with a mass to charge ratio (m/z) of 206±0.5 or 308±0.5. In some embodiments, one or more ions comprise a clonazepam precursor ion with a mass to charge ratio (m/z) of 316±0.5. In some embodiments, one or more ions comprise one or more fragment ions with a mass to charge ratio (m/z) of 214±0.5 or 270±0.5. In some embodiments, one or more ions comprise a flurazepam precursor ion with a mass to charge ratio (m/z) of 388±0.5. In some embodiments, one or more ions comprise one or more fragment ions with a mass to charge ratio (m/z) of 287.9±0.5 or 315±0.5. In some embodiments, one or more ions comprise a lorazepam precursor ion with a mass to charge ratio (m/z) of 321±0.5. In some embodiments, one or more ions comprise one or more fragment ions with a mass to charge ratio (m/z) of 229.1±0.5 or 331±0.5. In some embodiments, one or more ions comprise a flunitrazepam precursor ion with a mass to charge ratio (m/z) of 314±0.5. In some embodiments, one or more ions comprise one or more fragment ions with a mass to charge ratio (m/z) of 211±0.5 or 268±0.5. In some embodiments, one or more ions comprise a temazepam precursor ion with a mass to charge ratio (m/z) of 301.1±0.5. In some embodiments, one or more ions comprise one or more fragment ions with a mass to charge ratio (m/z) of 177±0.5 or 255±0.5. In some embodiments, one or more ions comprise a midazolam precursor ion with a mass to charge ratio (m/z) of 326±0.5. In some embodiments, one or more ions comprise one or more fragment ions with a mass to charge ratio (m/z) of 129±0.5 or 244±0.5. In some embodiments, one or more ions comprise an nordiazepam precursor ion with a mass to charge ratio (m/z) of 271±0.5. In some embodiments, one or more ions comprise one or more fragment ions with a mass to charge ratio (m/z) of 139.8±0.5 or 165±0.5. In some embodiments, one or more ions comprise an phenazepam precursor ion with a mass to charge ratio (m/z) of 351±0.5. In some embodiments, one or more ions comprise one or more fragment ions with a mass to charge ratio (m/z) of 185.9±0.5 or 206±0.5. In some embodiments, one or more ions comprise a chlordiazepam precursor ion with a mass to charge ratio (m/z) of 301±0.5. In some embodiments, one or more ions comprise one or more fragment ions with a mass to charge ratio (m/z) of 259±0.5 or 224±0.5. In some embodiments, one or more ions comprise a diazepam precursor ion with a mass to charge ratio (m/z) of 285±0.5. In some embodiments, one or more ions comprise one or more fragment ions with a mass to charge ratio (m/z) of 154±0.5 or 193±0.5. In some embodiments, one or more ions comprise a prazepam precursor ion with a mass to charge ratio (m/z) of 325±0.5. In some embodiments, one or more ions comprise one or more fragment ions with a mass to charge ratio (m/z) of 165±0.5 or 271±0.5. In some embodiments, one or more ions comprise a medazepam precursor ion with a mass to charge ratio (m/z) of 271±0.5. In some embodiments, one or more ions comprise one or more fragment ions with a mass to charge ratio (m/z) of 180±0.5 or 207.1±0.5.

In some embodiments, the analyte is an anti-epileptic drug. In some embodiments, the anti-epileptic drug is valproic acid, tiagabine, topiramate, levitiracetum, lamotrigine, lacosamide, ethosuximide, carbamazepine, eslicarbamazepine, 10,11-carbamazepine, phenobarbital, rufinamide, primidone, phenytoin, zonisamide, felbamate, gabapentin, or pregablin. In some embodiments, the anti-epileptic drug is selected from the group consisting of valproic acid, tiagabine, topiramate, levitiracetum, lamotrigine, lacosamide, ethosuximide, carbamazepine, eslicarbamazepine, 10,11-carbamazepine, phenobarbital, rufinamide, primidone, phenytoin, zonisamide, felbamate, gabapentin, and pregablin. In some embodiments, the anti-epileptic drug is extracted from a whole blood sample.

In some embodiments, one or more ions comprise a felbamate precursor ion with a mass to charge ratio (m/z) of 339±0.5. In some embodiments, one or more ions comprise one or more fragment ions with a mass to charge ratio (m/z) of 117.3±0.5 or 261±0.5. In some embodiments, one or more ions comprise an ethosuximide precursor ion with a mass to charge ratio (m/z) of 142±0.5. In some embodiments, one or more ions comprise one or more fragment ions with a mass to charge ratio (m/z) of 44.3±0.5 or 39.3±0.5. In some embodiments, one or more ions comprise a lacosamide precursor ion with a mass to charge ratio (m/z) of 251±0.5. In some embodiments, one or more ions comprise one or more fragment ions with a mass to charge ratio (m/z) of 91.2±0.5 or 65.2±0.5. In some embodiments, one or more ions comprise a lamotrigine precursor ion with a mass to charge ratio (m/z) of 256±0.5. In some embodiments, one or more ions comprise one or more fragment ions with a mass to charge ratio (m/z) of 211±0.5 or 145±0.5. In some embodiments, one or more ions comprise a topiramate precursor ion with a mass to charge ratio (m/z) of 338.2±0.5. In some embodiments, one or more ions comprise one or more fragment ions with a mass to charge ratio (m/z) of 78.2±0.5 or 96.2±0.5. In some embodiments, one or more ions comprise a gabapentin precursor ion with a mass to charge ratio (m/z) of 172.3±0.5. In some embodiments, one or more ions comprise one or more fragment ions with a mass to charge ratio (m/z) of 91.2±0.5 or 67.2±0.5. In some embodiments, one or more ions comprise an eslicarbazepine precursor ion with a mass to charge ratio (m/z) of 297.1±0.5. In some embodiments, one or more ions comprise one or more fragment ions with a mass to charge ratio (m/z) of 194±0.5 or 179±0.5. In some embodiments, one or more ions comprise a primidone precursor ion with a mass to charge ratio (m/z) of 219.8±0.5. In some embodiments, one or more ions comprise one or more fragment ions with a mass to charge ratio (m/z) of 79±0.5 or 135.2±0.5. In some embodiments, one or more ions comprise a pregabalin precursor ion with a mass to charge ratio (m/z) of 160.1±0.5. In some embodiments, one or more ions comprise one or more fragment ions with a mass to charge ratio (m/z) of 55.2±0.5 or 77.2±0.5. In some embodiments, one or more ions comprise a carbamazepine precursor ion with a mass to charge ratio (m/z) of 237±0.5. In some embodiments, one or more ions comprise one or more fragment ions with a mass to charge ratio (m/z) of 194.1±0.5 or 179±0.5. In some embodiments, one or more ions comprise a phenobarbital precursor ion with a mass to charge ratio (m/z) of 231±0.5. In some embodiments, one or more ions comprise one or more fragment ions with a mass to charge ratio (m/z) of 44.2±0.5 or 188.1±0.5. In some embodiments, one or more ions comprise an epoxide precursor ion with a mass to charge ratio (m/z) of 236.2±0.5. In some embodiments, one or more ions comprise one or more fragment ions with a mass to charge ratio (m/z) of 141.2±0.5 or 112.2±0.5. In some embodiments, one or more ions comprise a zonisamide precursor ion with a mass to charge ratio (m/z) of 213.2±0.5. In some embodiments, one or more ions comprise one or more fragment ions with a mass to charge ratio (m/z) of 77.2±0.5 or 102.1±0.5. In some embodiments, one or more ions comprise a tiagabine precursor ion with a mass to charge ratio (m/z) of 376.2±0.5. In some embodiments, one or more ions comprise one or more fragment ions with a mass to charge ratio (m/z) of 111.1±0.5 or 149.1±0.5. In some embodiments, one or more ions comprise a phenytoin precursor ion with a mass to charge ratio (m/z) of 253.1±0.5. In some embodiments, one or more ions comprise one or more fragment ions with a mass to charge ratio (m/z) of 104.2±0.5 or 182.2±0.5. In some embodiments, one or more ions comprise a levetiracetam precursor ion with a mass to charge ratio (m/z) of 171.2±0.5. In some embodiments, one or more ions comprise one or more fragment ions with a mass to charge ratio (m/z) of 126.2±0.5 or 69.2±0.5. In some embodiments, one or more ions comprise a valproic acid precursor ion with a mass to charge ratio (m/z) of 143±0.5. In some embodiments, one or more ions comprise one or more fragment ions with a mass to charge ratio (m/z) of 143±0.5. In some embodiments, one or more ions comprise a rufinamide precursor ion with a mass to charge ratio (m/z) of 239±0.5. In some embodiments, one or more ions comprise one or more fragment ions with a mass to charge ratio (m/z) of 127.2±0.5 or 261±0.5. In some embodiments, one or more ions comprise a primdone precursor ion with a mass to charge ratio (m/z) of 219±0.5. In some embodiments, one or more ions comprise one or more fragment ions with a mass to charge ratio (m/z) of 126±0.5 or 141±0.5. In some embodiments, one or more ions comprise a topiramate D12 precursor ion with a mass to charge ratio (m/z) of 350±0.5. In some embodiments, one or more ions comprise one or more fragment ions with a mass to charge ratio (m/z) of 78.2±0.5. In some embodiments, one or more ions comprise an epoxide D3 precursor ion with a mass to charge ratio (m/z) of 256±0.5. In some embodiments, one or more ions comprise one or more fragment ions with a mass to charge ratio (m/z) of 77±0.5. In some embodiments, one or more ions comprise a lamotrigine ¹³C₃ precursor ion with a mass to charge ratio (m/z) of 259±0.5. In some embodiments, one or more ions comprise one or more fragment ions with a mass to charge ratio (m/z) of 214±0.5. In some embodiments, one or more ions comprise a levetiracetam D6 precursor ion with a mass to charge ratio (m/z) of 177.2±0.5. In some embodiments, one or more ions comprise one or more fragment ions with a mass to charge ratio (m/z) of 132.2±0.5.

In some embodiments, the analyte is an immunosuppressant. In some embodiments, the immunosuppressant is cyclosporine A, sirolimus, tacrolimus, or everolimus. In some embodiments, the immunosuppressant is selected from the group consisting of cyclosporine A, sirolimus, tacrolimus, and everolimus. In some embodiments, the immunosuppressant is extracted from a whole blood sample.

In some embodiments, the analyte is a barbiturate. In some embodiments, the barbiturate is phenobarbitol, amobarbitol, butalbital, pentobarbitol, secobarbitol, or thiopental. In some embodiments, the barbiturate is selected from the group consisting of phenobarbitol, amobarbitol, butalbital, pentobarbitol, secobarbitol, and thiopental. In some embodiments, the barbiturate is extracted from a whole blood sample.

In some embodiments, one or more ions comprise a secobarbital precursor ion with a mass to charge ratio (m/z) of 237.0±0.5. In some embodiments, one or more ions comprise one or more fragment ions with a mass to charge ratio (m/z) of 42.0±0.5. In some embodiments, one or more ions comprise an ammobarbital precursor ion with a mass to charge ratio (m/z) of 225.0±0.5. In some embodiments, one or more ions comprise one or more fragment ions with a mass to charge ratio (m/z) of 182.0±0.5. In some embodiments, one or more ions comprise a pentobarbital precursor ion with a mass to charge ratio (m/z) of 225.6±0.5. In some embodiments, one or more ions comprise one or more fragment ions with a mass to charge ratio (m/z) of 42.0±0.5. In some embodiments, one or more ions comprise a thiopental precursor ion with a mass to charge ratio (m/z) of 241.0±0.5. In some embodiments, one or more ions comprise one or more fragment ions with a mass to charge ratio (m/z) of 57.9±0.5. In some embodiments, one or more ions comprise a phenobarbital precursor ion with a mass to charge ratio (m/z) of 231.0±0.5. In some embodiments, one or more ions comprise one or more fragment ions with a mass to charge ratio (m/z) of 42.0±0.5. In some embodiments, one or more ions comprise a butalbital precursor ion with a mass to charge ratio (m/z) of 223.1±0.5. In some embodiments, one or more ions comprise one or more fragment ions with a mass to charge ratio (m/z) of 42.1±0.5.

In some embodiments, the analyte is tamoxifen. In some embodiments, the analyte is a metabolite of tamoxifen. In some embodiments, said metabolite is norendoxifen. In some embodiments, said metabolite is endoxifen or N-Desmethyl-4-Hydroxy Tamoxifen. In some embodiments, said metabolite is 4′-Hydroxy Tamoxifen. In some embodiments, said metabolite is 4-Hydroxy Tamoxifen. In some embodiments, said metabolite is N-Desmethyl-4′-Hydroxy Tamoxifen. In some embodiments, said metabolite is N-Desmethyl Tamoxifen. In some embodiments, said metabolite is selected from the group consisting of norendoxifen, endoxifen, 4′-Hydroxy Tamoxifen, 4-Hydroxy Tamoxifen, N-Desmethyl-4′-Hydroxy Tamoxifen, and N-Desmethyl-4′-Hydroxy Tamoxifen. In some embodiments, tamoxifen or its metabolite is extracted from a whole blood sample.

In some embodiments, one or more ions comprise a tamoxifen precursor ion with a mass to charge ratio (m/z) of 372.2±0.5. In some embodiments, one or more ions comprise one or more fragment ions with a mass to charge ratio (m/z) of 72.14±0.5. In some embodiments, one or more ions comprise an endoxifen precursor ion with a mass to charge ratio (m/z) of 374.2±0.5. In some embodiments, one or more ions comprise one or more fragment ions with a mass to charge ratio (m/z) of 58.1±0.5. In some embodiments, one or more ions comprise a 4-hydroxy tamoxifen precursor ion with a mass to charge ratio (m/z) of 388.2±0.5. In some embodiments, one or more ions comprise one or more fragment ions with a mass to charge ratio (m/z) of 72.1±0.5. In some embodiments, one or more ions comprise an N-desmethyl-4′-hydroxy tamoxifen precursor ion with a mass to charge ratio (m/z) of 374.2±0.5. In some embodiments, one or more ions comprise one or more fragment ions with a mass to charge ratio (m/z) of 58.1±0.5. In some embodiments, one or more ions comprise a 4′-hydroxy tamoxifen precursor ion with a mass to charge ratio (m/z) of 388.2±0.5. In some embodiments, one or more ions comprise one or more fragment ions with a mass to charge ratio (m/z) of 72.1±0.5. In some embodiments, one or more ions comprise an N-desmethyl-4′-hydroxy tamoxifen precursor ion with a mass to charge ratio (m/z) of 358.2±0.5. In some embodiments, one or more ions comprise one or more fragment ions with a mass to charge ratio (m/z) of 58.1±0.5.

In some embodiments, the analyte is an oncology drug. In some embodiments, the analyte is anastrozole. In some embodiments, the analyte is letrozole. In some embodiments, the analyte is exemestane. In some embodiments, the analyte is selected from the group consisting of anastrozole, letrozole, and exemestane. In some embodiments, the oncology drug is extracted from a whole blood sample.

In some embodiments, the analyte is tetrahydrocannabinol (THC) or its metabolite. In some embodiments, THC is extracted from a urine sample.

In some embodiments, the extracted analyte is hydrolyzed. In some embodiments, the analyte is hydrolyzed prior to extraction.

In some embodiments, the collision energy is within the range of about 5 to 60 V. In some embodiments, the collision energy is within the range of about 5 to 60 V.

In another aspect, provided herein are methods for diagnosis of congenital adrenal hyperplasia in patients. In some embodiments, the methods of quantitation of endogenous steroids provided herein are used for diagnosing congenital adrenal hyperplasia.

In another aspect, provided herein are methods for detection or monitoring of THC use in an individual. In another aspect, provided herein are methods for detection or monitoring of barbiturate use in an individual. In another aspect, provided herein are methods for detection or monitoring of opiate use in an individual. In another aspect, provided herein are methods for detection or monitoring of benzodiazepine use in an individual.

In another aspect, provided herein are methods for detection or monitoring of anti-epileptic drug use in an individual. In another aspect, provided herein are methods for monitoring the anti-epileptic drug efficacy in an individual.

In another aspect, provided herein are methods for detection or monitoring of tamoxifen use in an individual. In another aspect, provided herein are methods for monitoring the tamoxifen efficacy in an individual.

In another aspect, certain methods presented herein utilize high resolution/high accuracy mass spectrometry to determine the amount of analyte in a sample. In some embodiments utilizing high accuracy/high resolution mass spectrometry, the methods include: (a) subjecting analyte from a sample to an ionization source under conditions suitable to generate ions, wherein the ions are detectable by mass spectrometry; and (b) determining the amount of one or more ions by high resolution/high accuracy mass spectrometry. In these embodiments, the amount of one or more ions determined in step (b) is related to the amount of analyte in the sample. In some embodiments, high resolution/high accuracy mass spectrometry is conducted at a FWHM of 10,000 and a mass accuracy of 50 ppm. In some embodiments, high resolution/high accuracy mass spectrometry is conducted with a high resolution/high accuracy time-of-flight (TOF) mass spectrometer. In some embodiments, the ionization conditions comprise ionization of analyte under acidic conditions. In some related embodiments, the acidic conditions comprise treatment of said sample with formic acid prior to ionization.

In any of the methods described herein, the sample may comprise a biological sample. In some embodiments, the biological sample may comprise a biological fluid such as urine, plasma, or serum. In some embodiments, the biological sample may comprise a sample from a human; such as from an adult male or female, or juvenile male or female, wherein the juvenile is under age 18, under age 15, under age 12, or under age 10. The human sample may be analyzed to diagnose or monitor a disease state or condition, or to monitor therapeutic efficacy of treatment of a disease state or condition. In some related embodiments, the methods described herein may be used to determine the amount of analyte in a biological sample when taken from a human.

In embodiments utilizing tandem mass spectrometry, tandem mass spectrometry may be conducted by any method known in the art, including for example, multiple reaction monitoring, precursor ion scanning, or product ion scanning.

In some embodiments, tandem mass spectrometry comprises fragmenting a precursor ion into one or more fragment ions. In embodiments where the amounts of two or more fragment ions are determined, the amounts may be subject to any mathematical manipulation known in the art in order to relate the measured ion amounts to the amount of analyte in the sample. For example, the amounts of two or more fragment ions may be summed as part of determining the amount of analyte in the sample.

In some embodiments, the high resolution/high accuracy mass spectrometry is conducted at a resolving power (FWHM) of greater than or equal to about 10,000, such as greater than or equal to about 15,000, such as greater than or equal to about 20,000, such as greater than or equal to about 25,000. In some embodiments, the high resolution/high accuracy mass spectrometry is conducted at an accuracy of less than or equal to about 50 ppm, such as less than or equal to about 20 ppm, such as less than or equal to about 10 ppm, such as less than or equal to about 5 ppm; such as less than or equal to about 3 ppm. In some embodiments, high resolution/high accuracy mass spectrometry is conducted at a resolving power (FWHM) of greater than or equal to about 10,000 and an accuracy of less than or equal to about 50 ppm. In some embodiments, the resolving power is greater than about 15,000 and the accuracy is less than or equal to about 20 ppm. In some embodiments, the resolving power is greater than or equal to about 20,000 and the accuracy is less than or equal to about 10 ppm; preferably resolving power is greater than or equal to about 20,000 and accuracy is less than or equal to about 5 ppm, such as less than or equal to about 3 ppm.

In some embodiments, the high resolution/high accuracy mass spectrometry may be conducted with an orbitrap mass spectrometer, a time of flight (TOF) mass spectrometer, or a Fourier transform ion cyclotron resonance mass spectrometer (sometimes known as a Fourier transform mass spectrometer).

Mass spectrometry (either tandem or high resolution/high accuracy) may be performed in positive ion mode. Alternatively, mass spectrometry may be performed in negative ion mode. Various ionization sources, including for example atmospheric pressure chemical ionization (APCI) or electrospray ionization (ESI), may be used to ionize the analyte.

In any method presented herein, a separately detectable internal standard may be provided in the sample, the amount of which is also determined in the sample. In embodiments utilizing a separately detectable internal standard, all or a portion of both the analyte of interest and the internal standard present in the sample is ionized to produce a plurality of ions detectable in a mass spectrometer, and one or more ions produced from each are detected by mass spectrometry. In these embodiments, the presence or amount of ions generated from the analyte of interest may be related to the presence of amount of analyte of interest in the sample by comparison to the amount of internal standard ions detected.

Alternatively, the amount of analyte in a sample may be determined by comparison to one or more external reference standards. Exemplary external reference standards include blank plasma or serum spiked with human or non-human analyte, a synthetic analyte analogue, or an isotopically labeled variant thereof.

### Sample Preparation for Mass Spectrometric Analysis

One method of sample purification that may be used prior to mass spectrometry is applying a sample to a solid-phase extraction (SPE) column under conditions where the analyte of interest is reversibly retained by the column packing material, while one or more other materials are not retained. In this technique, a first mobile phase condition can be employed where the analyte of interest is retained by the column, and a second mobile phase condition can subsequently be employed to remove retained material from the column, once the non-retained materials are washed through.

In some embodiments, analyte in a sample may be reversibly retained on a SPE column with a packing material comprising an alkyl bonded surface. For example, in some embodiments, a C-8 on-line SPE column (such as an Oasis HLB on-line SPE column/cartridge (2.1 mm×20 mm) from Phenomenex, Inc. or equivalent) may be used to enrich analyte prior to mass spectrometric analysis. In some embodiments, use of an SPE column is conducted with HPLC Grade 0.2% aqueous formic acid as a wash solution, and use of 0.2% formic acid in acetonitrile as an elution solution.

Another method of sample purification that may be used prior to mass spectrometry is liquid chromatography (LC). In liquid chromatography techniques, an analyte may be purified by applying a sample to a chromatographic analytical column under mobile phase conditions where the analyte of interest elutes at a differential rate in comparison to one or more other materials. Such procedures may enrich the amount of one or more analytes of interest relative to one or more other components of the sample.

Certain methods of liquid chromatography, including HPLC, rely on relatively slow, laminar flow technology. Traditional HPLC analysis relies on column packing in which laminar flow of the sample through the column is the basis for separation of the analyte of interest from the sample. The skilled artisan will understand that separation in such columns is a partition process and may select LC, including HPLC, instruments and columns that are suitable for use with C peptide. The chromatographic analytical column typically includes a medium (i.e., a packing material) to facilitate separation of chemical moieties (i.e., fractionation). The medium may include minute particles. The particles typically include a bonded surface that interacts with the various chemical moieties to facilitate separation of the chemical moieties. One suitable bonded surface is a hydrophobic bonded surface such as an alkyl bonded or a cyano bonded surface. Alkyl bonded surfaces may include C-4, C-8, C-12, or C-18 bonded alkyl groups. In some embodiments, the chromatographic analytical column is a monolithic C-18 column. The chromatographic analytical column includes an inlet port for receiving a sample and an outlet port for discharging an effluent that includes the fractionated sample. The sample may be supplied to the inlet port directly, or from a SPE column, such as an on-line SPE column or a TFLC column. In some embodiments, an on-line filter may be used ahead of the SPE column and or HPLC column to remove particulates and phospholipids in the samples prior to the samples reaching the SPE and/or TFLC and/or HPLC columns.

In one embodiment, the sample may be applied to the LC column at the inlet port, eluted with a solvent or solvent mixture, and discharged at the outlet port. Different solvent modes may be selected for eluting the analyte(s) of interest. For example, liquid chromatography may be performed using a gradient mode, an isocratic mode, or a polytypic (i.e. mixed) mode. During chromatography, the separation of materials is effected by variables such as choice of eluent (also known as a “mobile phase”), elution mode, gradient conditions, temperature, etc.

In some embodiments, analyte in a sample is enriched with HPLC. This HPLC may be conducted with a monolithic C-18 column chromatographic system, for example, an Onyx Monolithic C-18 column from Phenomenex Inc. (50×2.0 mm), or equivalent. In certain embodiments, HPLC is performed using HPLC Grade 0.2% aqueous formic acid as solvent A, and 0.2% formic acid in acetonitrile as solvent B.

By careful selection of valves and connector plumbing, two or more chromatography columns may be connected as needed such that material is passed from one to the next without the need for any manual steps. In preferred embodiments, the selection of valves and plumbing is controlled by a computer pre-programmed to perform the necessary steps. Most preferably, the chromatography system is also connected in such an on-line fashion to the detector system, e.g., an MS system. Thus, an operator may place a tray of samples in an autosampler, and the remaining operations are performed under computer control, resulting in purification and analysis of all samples selected.

In some embodiments, TFLC may be used for purification of analyte prior to mass spectrometry. In such embodiments, samples may be extracted using a TFLC column which captures the analyte. The analyte is then eluted and transferred on-line to an analytical HPLC column. For example, sample extraction may be accomplished with a TFLC extraction cartridge with a large particle size (50 μm) packing. Sample eluted off of this column may then be transferred on-line to an HPLC analytical column for further purification prior to mass spectrometry. Because the steps involved in these chromatography procedures may be linked in an automated fashion, the requirement for operator involvement during the purification of the analyte can be minimized. This feature may result in savings of time and costs, and eliminate the opportunity for operator error.

In some embodiments, one or more of the above purification techniques may be used in parallel for purification of analyte to allow for simultaneous processing of multiple samples.

### Detection and Quantitation of Analyte by Mass Spectrometry

Mass spectrometry is performed using a mass spectrometer, which includes an ion source for ionizing the fractionated sample and creating charged molecules for further analysis. In various embodiments, analyte may be ionized by any method known to the skilled artisan. For example, ionization of analyte may be performed by electron ionization, chemical ionization, electrospray ionization (ESI), photon ionization, atmospheric pressure chemical ionization (APCI), photoionization, atmospheric pressure photoionization (APPI), Laser diode thermal desorption (LDTD), fast atom bombardment (FAB), liquid secondary ionization (LSI), matrix assisted laser desorption ionization (MALDI), field ionization, field desorption, thermospray/plasmaspray ionization, surface enhanced laser desorption ionization (SELDI), inductively coupled plasma (ICP) and particle beam ionization. The skilled artisan will understand that the choice of ionization method may be determined based on the analyte to be measured, type of sample, the type of detector, the choice of positive versus negative mode, etc. analyte may be ionized in positive or negative mode. In preferred embodiments, analyte is ionized by ESI in positive ion mode.

In mass spectrometry techniques generally, after the sample has been ionized, the positively or negatively charged ions thereby created may be analyzed to determine a mass to charge ratio (m/z). Various analyzers for determining m/z include quadrupole analyzers, ion traps analyzers, time-of-flight analyzers, Fourier transform ion cyclotron resonance mass analyzers, and orbitrap analyzers. Some exemplary ion trap methods are described in Bartolucci, et al., Rapid Commun. Mass Spectrom. 2000, 14:967-73.

The ions may be detected using several detection modes. For example, selected ions may be detected, i.e. using a selective ion monitoring mode (SIM), or alternatively, mass transitions resulting from collision induced dissociation or neutral loss may be monitored, e.g., multiple reaction monitoring (MRM) or selected reaction monitoring (SRM). In some embodiments, the mass-to-charge ratio is determined using a quadrupole analyzer. In a “quadrupole” or “quadrupole ion trap” instrument, ions in an oscillating radio frequency field experience a force proportional to the DC potential applied between electrodes, the amplitude of the RF signal, and the mass/charge ratio. The voltage and amplitude may be selected so that only ions having a particular mass/charge ratio travel the length of the quadrupole, while all other ions are deflected. Thus, quadrupole instruments may act as both a “mass filter” and as a “mass detector” for the ions injected into the instrument.

As ions collide with the detector they produce a pulse of electrons that are converted to a digital signal. The acquired data is relayed to a computer, which plots counts of the ions collected versus time. The resulting mass chromatograms are similar to chromatograms generated in traditional HPLC-MS methods. The areas under the peaks corresponding to particular ions, or the amplitude of such peaks, may be measured and correlated to the amount of the analyte of interest. In certain embodiments, the area under the curves, or amplitude of the peaks, for fragment ion(s) and/or precursor ions are measured to determine the amount of analyte. The relative abundance of a given ion may be converted into an absolute amount of the original analyte using calibration standard curves based on peaks of one or more ions of an internal or external molecular standard.

One may enhance the resolution of MS techniques employing certain mass spectrometric analyzers through “tandem mass spectrometry,” or “MS/MS”. In this technique, a precursor ion (also called a parent ion) generated from a molecule of interest can be filtered in an MS instrument, and the precursor ion subsequently fragmented to yield one or more fragment ions (also called daughter ions or product ions) that are then analyzed in a second MS procedure. By careful selection of precursor ions, only ions produced by certain analytes are passed to the fragmentation chamber, where collisions with atoms of an inert gas produce the fragment ions. Because both the precursor and fragment ions are produced in a reproducible fashion under a given set of ionization/fragmentation conditions, the MS/MS technique may provide an extremely powerful analytical tool. For example, the combination of filtration/fragmentation may be used to eliminate interfering substances, and may be particularly useful in complex samples, such as biological samples. In certain embodiments, a mass spectrometric instrument with multiple quadrupole analyzers (such as a triple quadrupole instrument) is employed to conduct tandem mass spectrometric analysis.

In certain embodiments using a MS/MS technique, precursor ions are isolated for further fragmentation, and collision activated dissociation (CAD) is used to generate fragment ions from the precursor ions for further detection. In CAD, precursor ions gain energy through collisions with an inert gas, and subsequently fragment by a process referred to as “unimolecular decomposition.” Sufficient energy must be deposited in the precursor ion so that certain bonds within the ion can be broken due to increased vibrational energy.

In some embodiments, analyte in a sample is detected and/or quantified using MS/MS as follows. Analyte is enriched in a sample by first subjecting the sample to SPE, then to liquid chromatography, preferably HPLC; the flow of liquid solvent from a chromatographic analytical column enters the heated nebulizer interface of an MS/MS analyzer; and the solvent/analyte mixture is converted to vapor in the heated charged tubing of the interface. During these processes, the analyte is ionized. The ions, e.g. precursor ions, pass through the orifice of the instrument and enter the first quadrupole. Quadrupoles 1 and 3 (Q1 and Q3) are mass filters, allowing selection of ions (i.e., selection of “precursor” and “fragment” ions in Q1 and Q3, respectively) based on their mass to charge ratio (m/z). Quadrupole 2 (Q2) is the collision cell, where ions are fragmented. The first quadrupole of the mass spectrometer (Q1) selects for molecules with the m/z of an analyte ion. Precursor ions with the correct m/z are allowed to pass into the collision chamber (Q2), while unwanted ions with any other m/z collide with the sides of the quadrupole and are eliminated. Precursor ions entering Q2 collide with neutral gas molecules (such as Argon molecules) and fragment. The fragment ions generated are passed into quadrupole 3 (Q3), where the fragment ions are selected for detection.

Alternate modes of operating a tandem mass spectrometric instrument that may be used in certain embodiments include product ion scanning and precursor ion scanning. For a description of these modes of operation, see, e.g., E. Michael Thurman, et al., Chromatographic-Mass Spectrometric Food Analysis for Trace Determination of Pesticide Residues, Chapter 8 (Amadeo R. Fernandez-Alba, ed., Elsevier 2005) (387).

In other embodiments, a high resolution/high accuracy mass analyzer may be used for quantitative analysis of analyte according to methods of the present invention. To achieve acceptable precision for quantitative results, the mass spectrometer must be capable of exhibiting a resolving power (FWHM) of 10,000 or more, with accuracy of about 50 ppm or less for the ions of interest; preferably the mass spectrometer exhibits a resolving power (FWHM) of 18,000 or better, with accuracy of about 5 ppm or less; such as a resolving power (FWHM) of 20,000 or better and accuracy of about 3 ppm or less; such as a resolving power (FWHM) of 25,000 or better and accuracy of about 3 ppm or less. Three exemplary analyzers capable of exhibiting the requisite level of performance for analyte ions are orbitrap mass analyzers, certain TOF mass analyzers, and Fourier transform ion cyclotron resonance mass analyzers.

Elements found in biological active molecules, such as carbon, oxygen, and nitrogen, naturally exist in a number of different isotopic forms. For example, most carbon is present as ¹²C, but approximately 1% of all naturally occurring carbon is present as ¹³C. Thus, some fraction of naturally occurring molecules containing at least one carbon atom will contain at least one ¹³C atom. Inclusion of naturally occurring elemental isotopes in molecules gives rise to multiple molecular isotopic forms. The difference in masses of molecular isotopic forms is at least 1 atomic mass unit (amu). This is because elemental isotopes differ by at least one neutron (mass of one neutron 1 amu). When molecular isotopic forms are ionized to multiply charged states, the mass distinction between the isotopic forms can become difficult to discern because mass spectrometric detection is based on the mass to charge ratio (m/z). For example, two isotopic forms differing in mass by 1 amu that are both ionized to a 5+ state will exhibit differences in their m/z of only 0.2. High resolution/high accuracy mass spectrometers are capable of discerning between isotopic forms of highly multiply charged ions (such as ions with charges of ±2, ±3, ±4, ±5, or higher).

Due to naturally occurring elemental isotopes, multiple isotopic forms typically exist for every molecular ion (each of which may give rise to a separately detectable spectrometric peak if analyzed with a sensitive enough mass spectrometric instrument). The m/z ratios and relative abundances of multiple isotopic forms collectively comprise an isotopic signature for a molecular ion. In some embodiments, the m/z ratios and relative abundances for two or more molecular isotopic forms may be utilized to confirm the identity of a molecular ion under investigation. In some embodiments, the mass spectrometric peak from one or more isotopic forms is used to quantitate a molecular ion. In some related embodiments, a single mass spectrometric peak from one isotopic form is used to quantitate a molecular ion. In other related embodiments, a plurality of isotopic peaks are used to quantitate a molecular ion. In these later embodiments, the plurality of isotopic peaks may be subject to any appropriate mathematical treatment. Several mathematical treatments are known in the art and include, but are not limited to summing the area under multiple peaks, or averaging the response from multiple peaks. However, that the precise masses observed for isotopic variants of any ion may vary slightly because of instrumental variance.

In some embodiments, the relative abundance of one or more ion is measured with a high resolution/high accuracy mass spectrometer in order to qualitatively assess the amount of analyte in the sample. Use of high resolution orbitrap analyzers has been reported for qualitative and quantitative analyses of various analytes. See, e.g., U.S. Patent Application Pub. No. 2008/0118932 (filed Nov. 9, 2007); Bredehöft, et al., Rapid Commun. Mass Spectrom., 2008, 22:477-485; Le Breton, et al., Rapid Commun. Mass Spectrom., 2008, 22:3130-36; Thevis, et al., Mass Spectrom. Reviews, 2008, 27:35-50; Thomas, et al., J. Mass Spectrom., 2008, 43:908-15; Schenk, et al., BMC Medical Genomics, 2008, 1:41; and Olsen, et al., Nature Methods, 2007, 4:709-12.

The results of an analyte assay may be related to the amount of the analyte in the original sample by numerous methods known in the art. For example, given that sampling and analysis parameters are carefully controlled, the relative abundance of a given ion may be compared to a table that converts that relative abundance to an absolute amount of the original molecule. Alternatively, external standards may be run with the samples, and a standard curve constructed based on ions generated from those standards. Using such a standard curve, the relative abundance of a given ion may be converted into an absolute amount of the original molecule. In certain preferred embodiments, an internal standard is used to generate a standard curve for calculating the quantity of analyte. Methods of generating and using such standard curves are well known in the art and one of ordinary skill is capable of selecting an appropriate internal standard. For example, in preferred embodiments one or more forms of isotopically labeled analyte may be used as internal standards. Numerous other methods for relating the amount of an ion to the amount of the original molecule will be well known to those of ordinary skill in the art.

As used herein, an “isotopic label” produces a mass shift in the labeled molecule relative to the unlabeled molecule when analyzed by mass spectrometric techniques. Examples of suitable labels include deuterium (²H), ¹³C, and ¹⁵N. One or more isotopic labels can be incorporated at one or more positions in the molecule and one or more kinds of isotopic labels can be used on the same isotopically labeled molecule.

One or more steps of any of the above described methods may be performed using automated machines. In certain embodiments, one or more purification steps are performed on-line, and more preferably all of the purification and mass spectrometry steps may be performed in an on-line fashion.

The following Examples serve to illustrate the invention. These Examples are in no way intended to limit the scope of the methods.

### EXAMPLES

### Example 1

### Mass Spectrometric Assay of Steroids

Patient samples were extracted directly from the 20 uL MITRA® tips. The tips were directly placed on the NUNC® 96-deep well plate. 500 uL extraction solvent (1M NH4OH in 50/50 Methanol/Ethyl Acetate) and 50 uL of internal standard (containing the stable isotope) and the extraction buffer were then added to the each well. The plate was mixed at room temperature for one hour prior to drying down under nitrogen. After the dry down step, the samples were brought back into solution by adding aqueous acid and organic solution (200 uL 0.1% FA in 50/50 Water/Methanol) to each well. The plate is mixed and then filtered. 100 uL of the filtrate was injected into the LC-MS/MS system with APCI (atmospheric pressure chemical ionization) source in positive ion mode. The following reagents were used: Mobile Phase A—0.1% Formic Acid in Water; Mobile Phase B—80/20 Methanol/Acetonitrile; Extraction Solvent: 1M Ammonium Hydroxide in 50/50 Methanol/Ethyl acetate.

ARIA® TX-4 System from Thermo Scientific was used for liquid chromatography and separation was accomplished by a reverse phase analytical column (KINETEX® C18) HPLC column. The detector used was QTRAP® 6500 from AB Sciex.

The following steroids were detected and quantitated underivatized using one 20 uL MITRA® tip or two 6 mm punch from DBS: cortisol, cortisone, progesterone, 17-hydroxyprogesterone, androstenedione, testosterone, dehydroepiandrosterone, corticosterone, deoxycorticosterone, 11-deoxycortisol, pregnenolone, 17-hydroxypregnenolone & 21-deoxycortisol. FIG. 1.

The following mass transitions were used to analyze by mass spectrometry.

| | Parent | Product(s) | Retention Time |
|---|---|---|---|
| Compound | (m/z) | (m/z) | (min) |
| Cortisone | 361.4 | 121.2, 163.2 | 0.94 |
| Cortisol | 363.4 | 121.1, 267.2 | 1.28 |
| 21-Deoxycortisol | 347.3 | 121.1, 269.2 | 2.32 |
| Corticosterone | 347.4 | 121.1, 311.3 | 2.65 |
| 11-Deoxycortisol | 347.4 | 97.1, 109.1 | 2.91 |
| Androstenedione | 287.4 | 97.1, 109.1 | 4.17 |
| 11-Deoxycorticosterone | 331.4 | 97.1, 109.1 | 2.91 |
| Testosterone | 289.4 | 97.1, 109.1 | 4.58 |
| 17-Hydroxyprogesterone | 331.4 | 97.1, 109.1 | 5.04 |
| Progesterone | 315.3 | 97.1, 109.1 | 5.87 |
| Cortisone-d7 | 369.4 | 169.2 | 0.94 |
| Cortisol-d4 | 367.4 | 121.0 | 1.28 |
| Corticosterone-d4 | 351.1 | 121.1 | 2.65 |
| 11-Deoxycortisol-13C3 | 350.4 | 100.1 | 2.91 |
| Androstenedione-13C3 | 290.4 | 100.1 | 4.17 |
| Testosterone-13C3 | 292.4 | 112.1 | 4.58 |
| 17-Hydroxyprogesterone- | 334.3 | 100.0 | 5.04 |
| 13C3 | | | |
| Progesterone-13C3 | 318.5 | 100.1 | 5.87 |

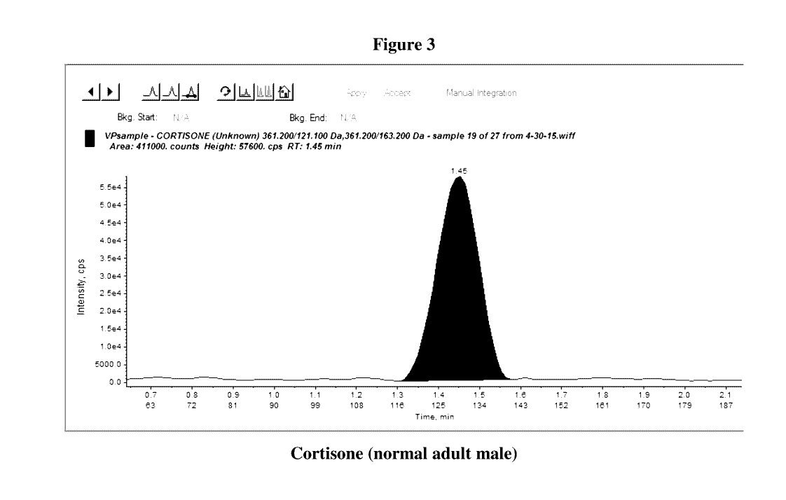

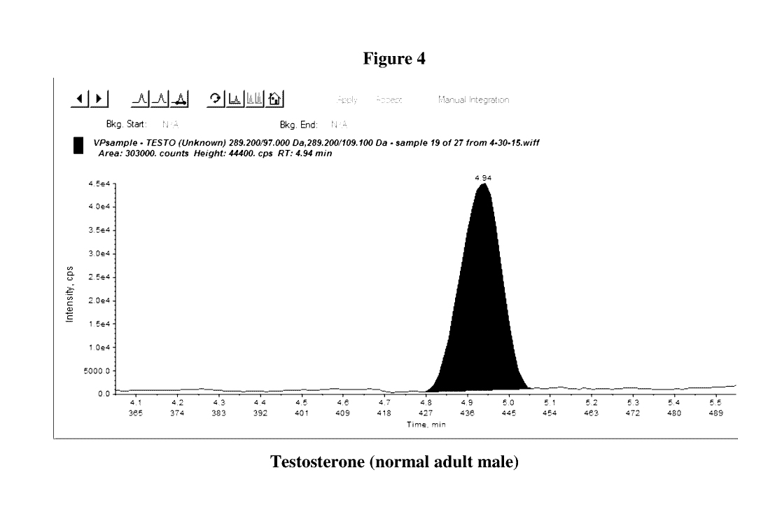

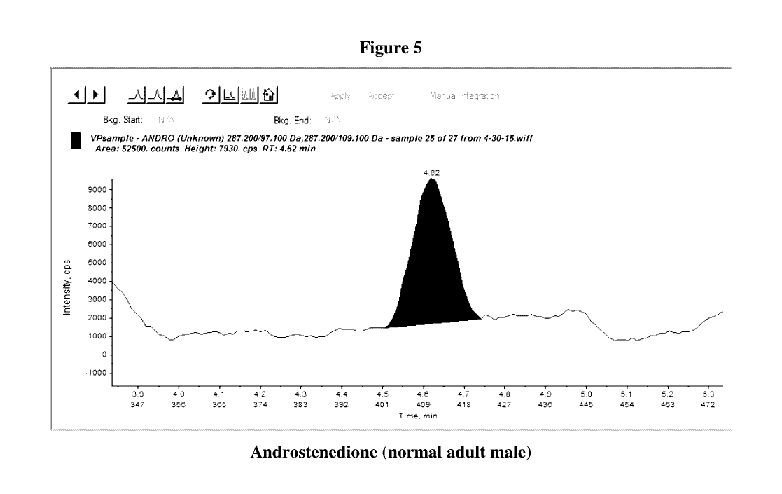

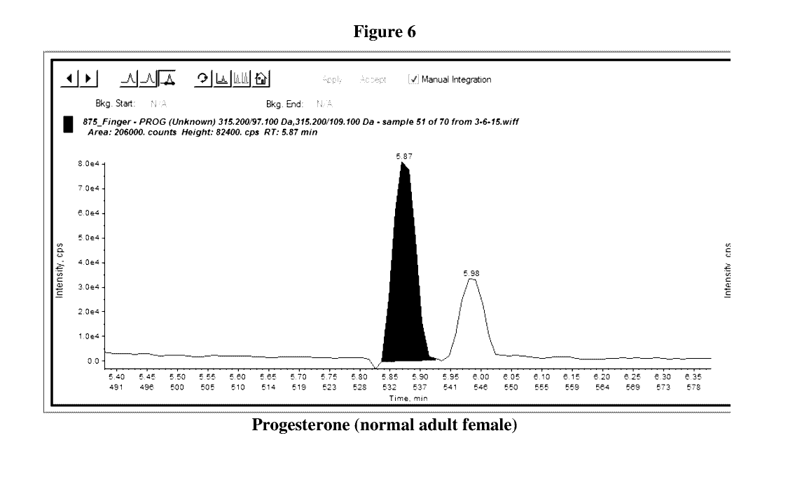

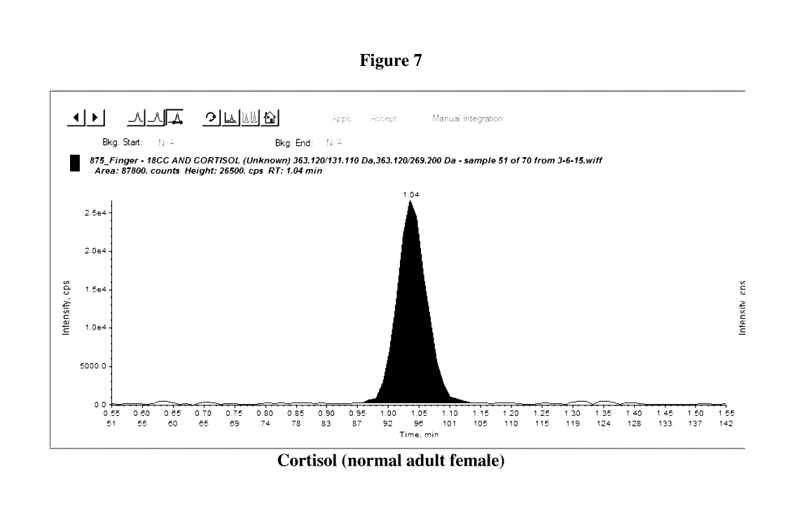

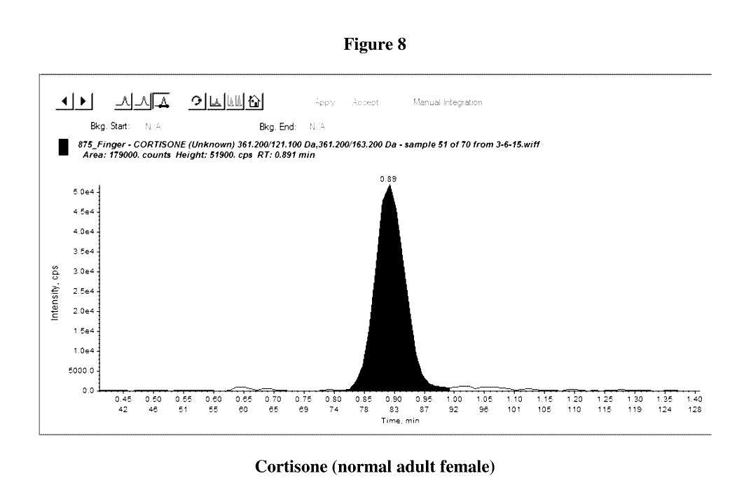

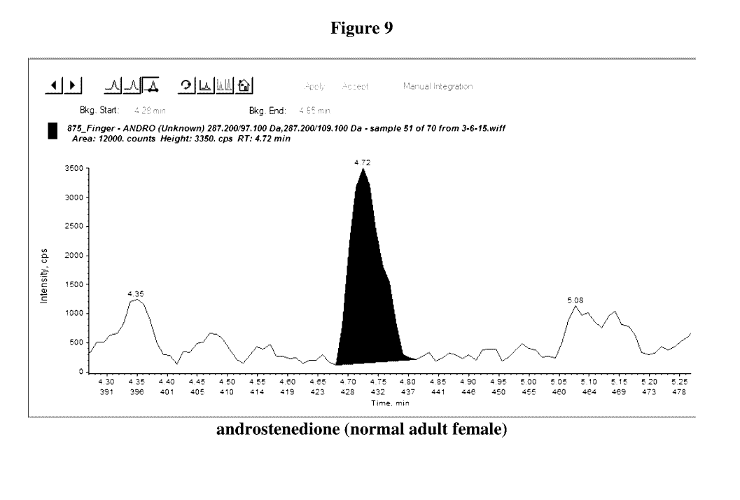

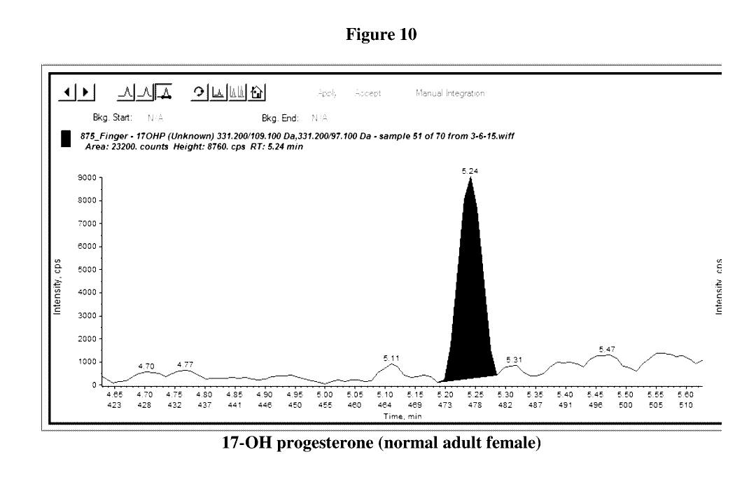

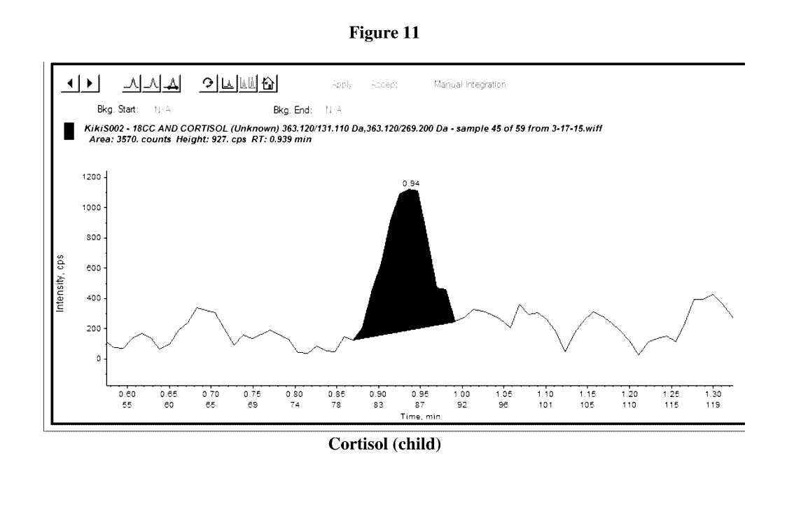

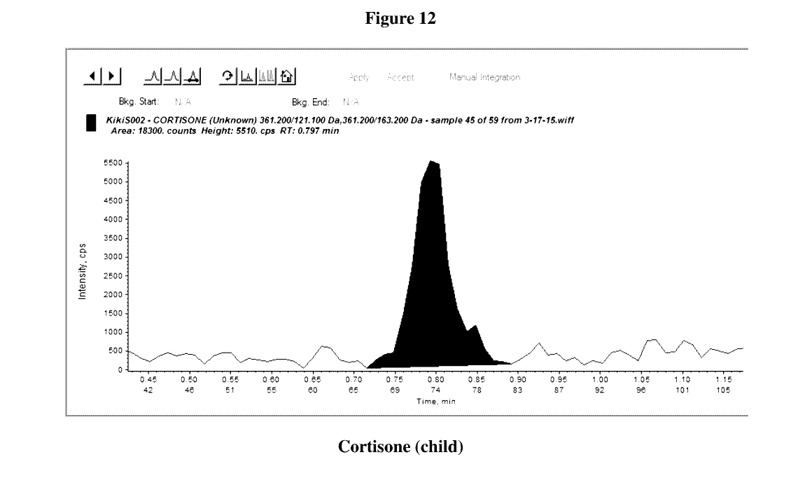

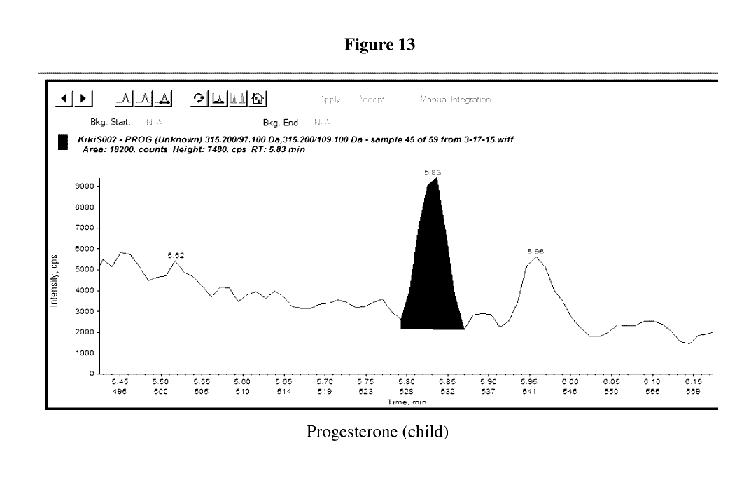

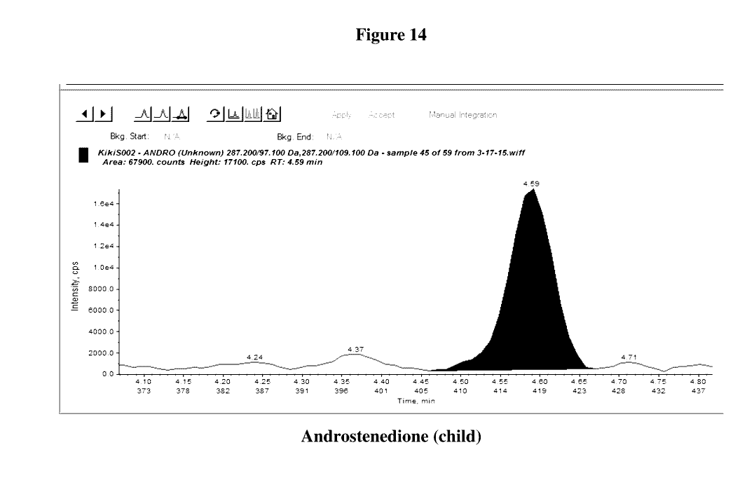

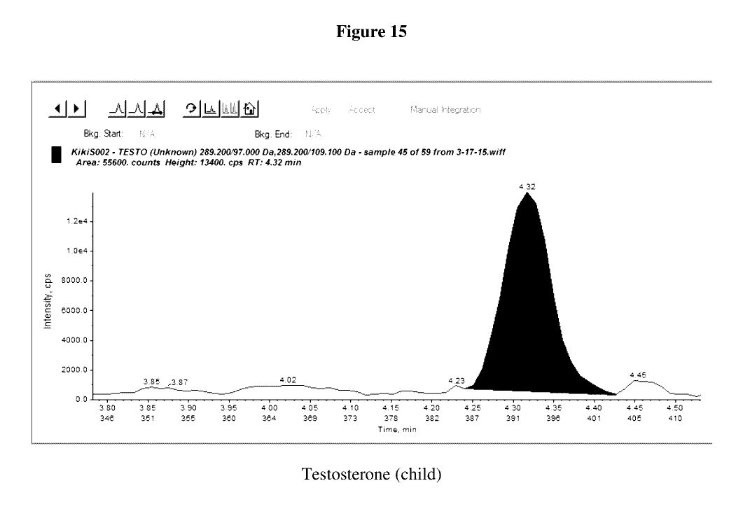

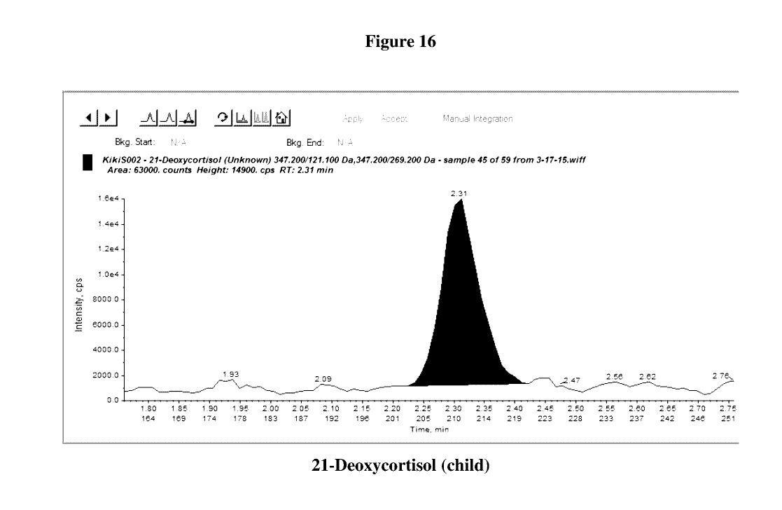

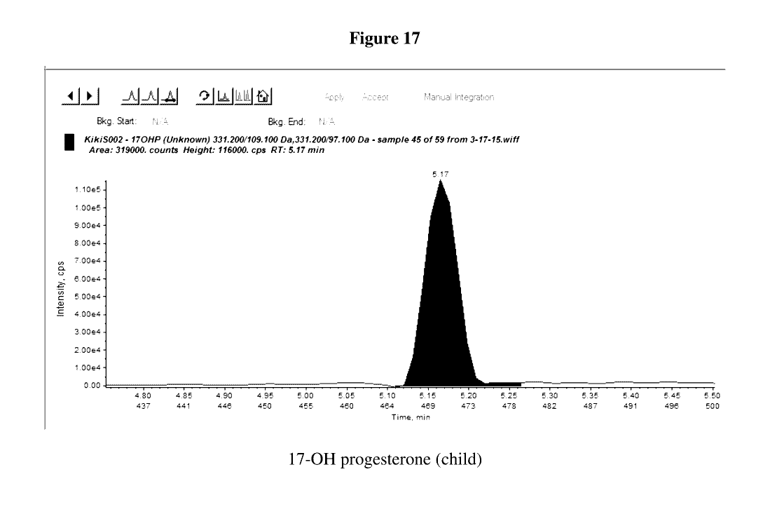

FIGS. 2-17 show levels of various steroids in adult male, adult female, and child.

Table 1 shows distinguishing characteristics of the congenital adrenal hyperplasia enzyme deficiencies:

TABLE 1
Distinguishing Characteristics^{a} of the Congenital Adrenal
Hyperplasia Enzyme Deficiencies^{1,2} [return to comtents]

| | 21α-Hydroxylase | | 11β-Hydroxylase | 17α-Hydroxylase | 3β-Hydroxysteroid |
|---|---|---|---|---|---|
| | (P450c21) | | (P450c11) | (P450c17) | Dehydrogenase |
| | Classic | Nonclassic | Classic | Classic | Classic |
| Gene | CYP21A2 | CYP21A2 | CYP11B1 | CYP17A1 | HSD3B2 |
| Incidence^{b} | 1:10,000-20,000 | 1:1000 | 1:100,000 | Rare | Rare |
| Elevated | 17-OHP | 17-OHP | DOC | DOC | DHEA |
| Steroids | Progesterone | Exaggerated | 11-Deoxy- | Corticosterone | 17-OH pregneno- |
| | Androstenedione | androstene- | cortisol | Progesterone | lone |
| | DHEA | dione, DHEA, | | | Pregnenolone |
| | | and 17-OHP | | | |
| | | response | | | |
| | | to ACTH | | | |
| Decreased | Aldosterone | None | Cortisol | Cortisol | Cortisol |
| Steroids | Corticosterone | | Corticosterone | Aldosterone | Aldosterones |
| | (salt-wasting) | | Aldosterone | | |
| | Cortisol (simple | | | | |
| | virilizing) | | | | |
| Age at | Infancy | Childhood/ | Neonatal | Puberty | Early infancy |
| Diagnosis | | puberty | to adult | | (severe) |
| | | | | | Post puberty |
| | | | | | (mild) |
| Genitalia | | | | | |
| Females | Virilized | ±Mild | Mild/severe | No puberty | Mild |
| (X, X) | | virilization | virilization | | virilization |
| Males | Normal | Normal | Normal | Ambiguous | Ambiguous |
| (X, Y) | | | | | |
| Androgens | ↑ | ↑ | ↑ | ↓ | ↓ in males |
| | | | | | ↑ in females |
| Estrogens | ↓ | ↓ | ↓ in females | ↓ | ↓ |
| Na⁺ | ↓ in salt-wasting | Normal | ↑ | ↑ | ↓ |
| K⁻ | ↑ | Normal | ↓ | ↓ | ↑ |
| Blood | ↓ | Normal | ↑ | ↑ | ↓ |
| Pressure | | | | | |

Analytical Sensitivity: The limit of quantitation (LOQ) is the point where measurements become quantitatively meaningful. The acceptability criteria for the LOQ is defined as the lowest, reproducible concentration at which the coefficient of variation (CV) is ≦20%. To determine the preliminary LOQ, several replicates of samples varying in concentration were run over several days. Preliminary analytical measurable range was determined in a linear range study.

TABLE 2

| Analyte | Linear Range | Units | LOQ | Units |
|---|---|---|---|---|
| Cortisone | 0.5-10 | ug/dL | 0.2 | ug/dL |
| Cortisol | 1-50 | ug/dL | 0.25 | ug/dL |
| 21-Deoxycortisol | 100-10,000 | ng/dL | 75 | ng/dL |
| Corticosterone | 200-10,000 | ng/dL | 100 | ng/dL |
| 11-Deoxycortisol | 100-10,000 | ng/dL | 50 | ng/dL |
| Androstenedione | 50-10,000 | ng/dL | 40 | ng/dL |
| 11-Deoxycorticosterone | 100-10,000 | ng/dL | 50 | ng/dL |
| Testosterone | 50-10,000 | ng/dL | 40 | ng/dL |
| 17-Hydroxyprogesterone | 50-10,000 | ng/dL | 40 | ng/dL |
| Progesterone | 1.5-100 | ng/mL | 1 | ng/mL |

Table 3 shows differential diagnosis of enzymatic deficiencies causing classic congenital adrenal hyperplasia:

TABLE 3
Differential Diagnosis of Enzymatic Deficiencies Causing Classic
Congenital Adrenal Hyperplasia^{1,2} [return to contents]

| | | | | 3-Hydroxysteroid |
|---|---|---|---|---|
| | 21-Hydroxylase | 11β-Hydroxylase | 17-Hydroxylase | Dehydrogenase |
| Steroid^{a} | Deficiency | Deficiency | Deficiency | Deficiency |
| Androstenedione | ↑ | ↑ | | ↓ |
| Cortisol | ↓ | ↓ | ↓ | ↓ |
| Deoxycorticosterone | ↓ | ↑ | ↑ | |
| 11-Deoxycortisol | ↓ | ↑ | | |
| DHEA | ↑ | | | ↑ |
| 17-Hydroxypregnenolone | ↑ | | ↓ | ↑ |
| 17-Hydroxyprogesterone | ↑ | ±↑ | ↓ | |
| Progesterone | ↑ | | ↑ | ↓ |
| Testosterone (total) | ↑ | ↑ | ↓ | ↓ |
| Precursor product ratio | >10^{b} | >15^{c} | >10^{d} | >10^{e} |

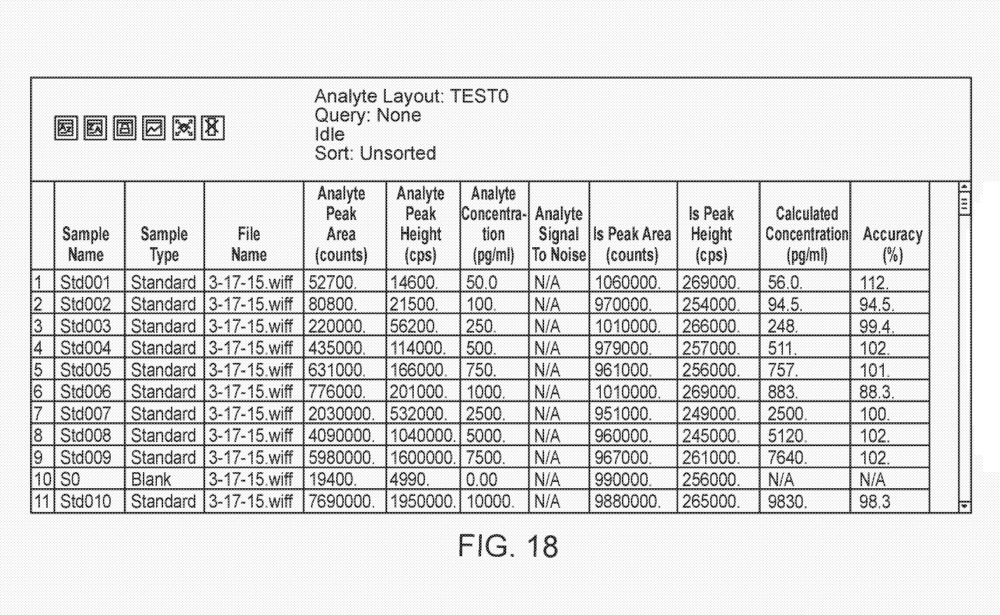

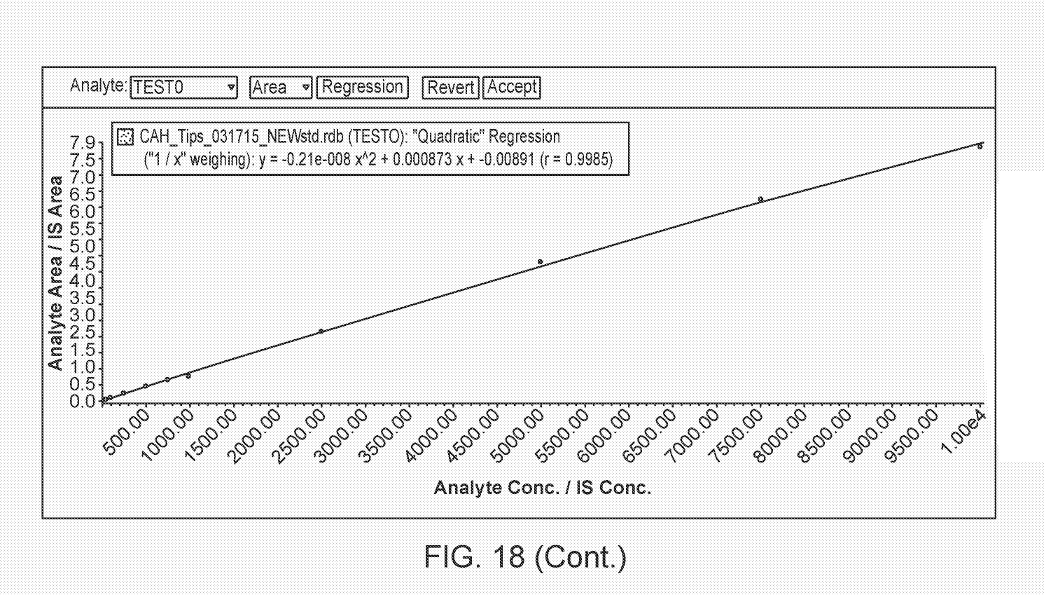

FIG. 18 shows standard linearity of testosterone between 50-10,000 ng/dL.

### Example 2

### Oncology Drugs

In this assay, 20 uL MITRA® tips were used to collect patient samples. The tips were pre-soaked in internal standard and dried for 2-24 hours.

The tips were soaked in calibration standards. The samples were eluted in 500 uL of elution buffer and dried down. The samples were then resuspended in 200 uL of loading buffer. 90 uL of samples were injected into the LC-MS/MS for quantitation.

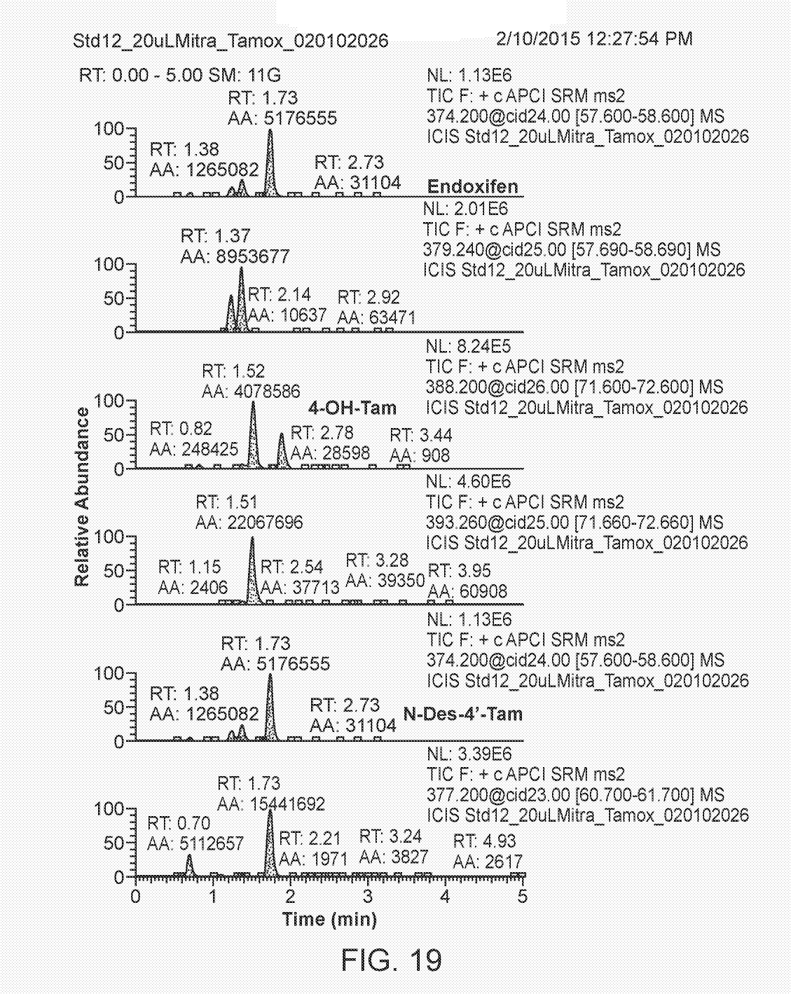

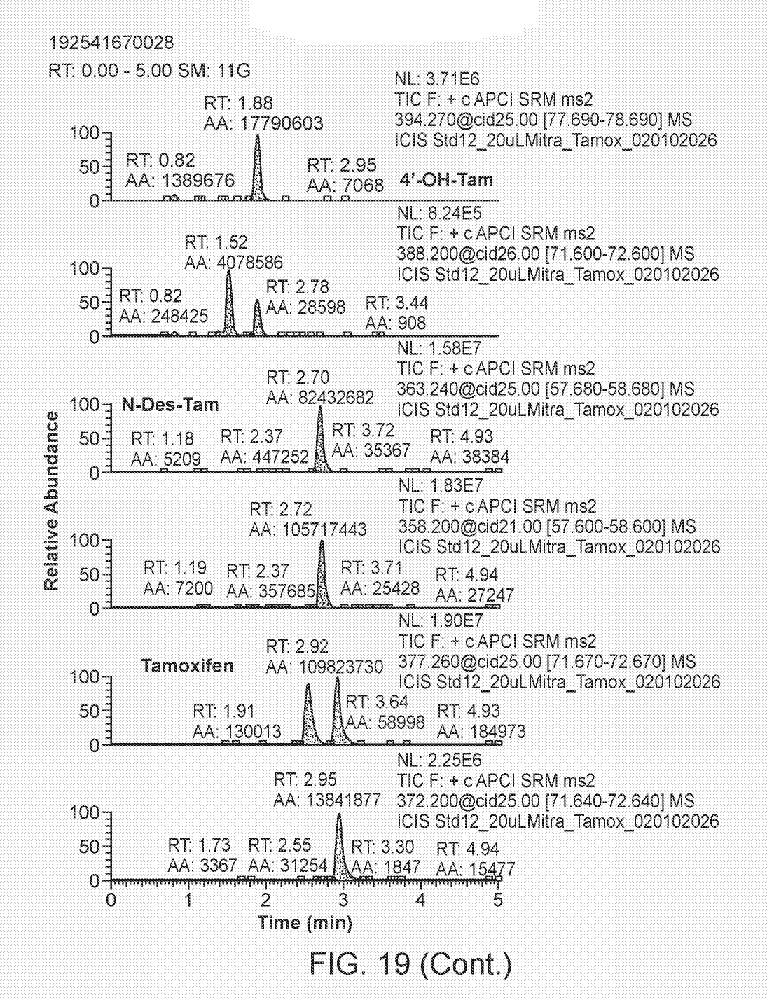

FIG. 19 shows chromatogram of tamoxifen and its metabolites.

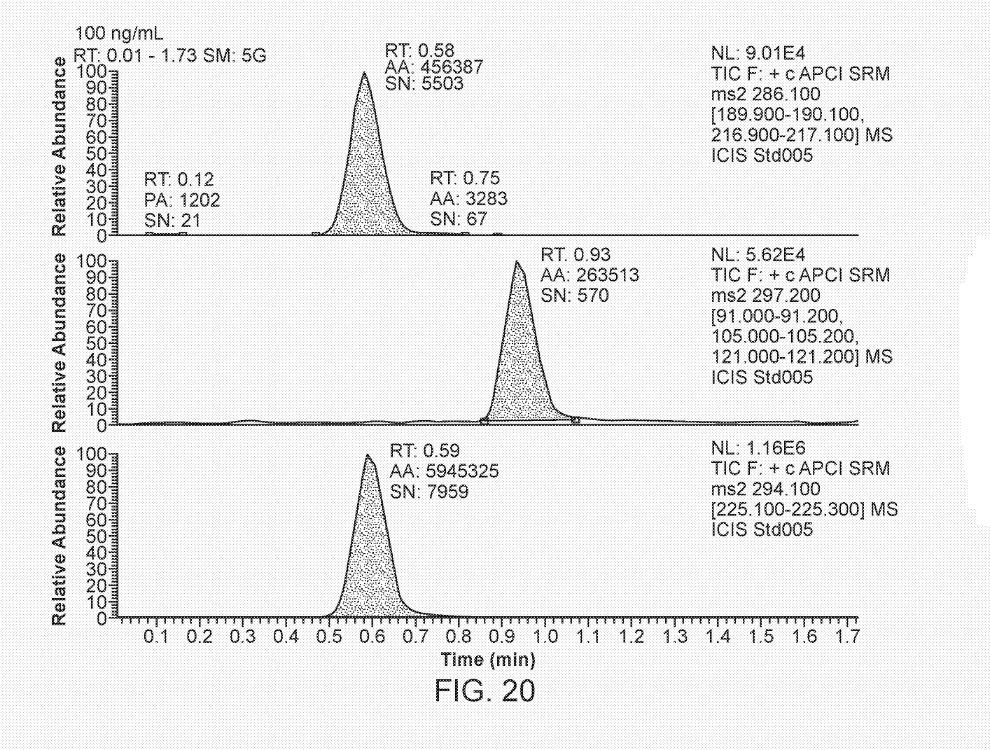

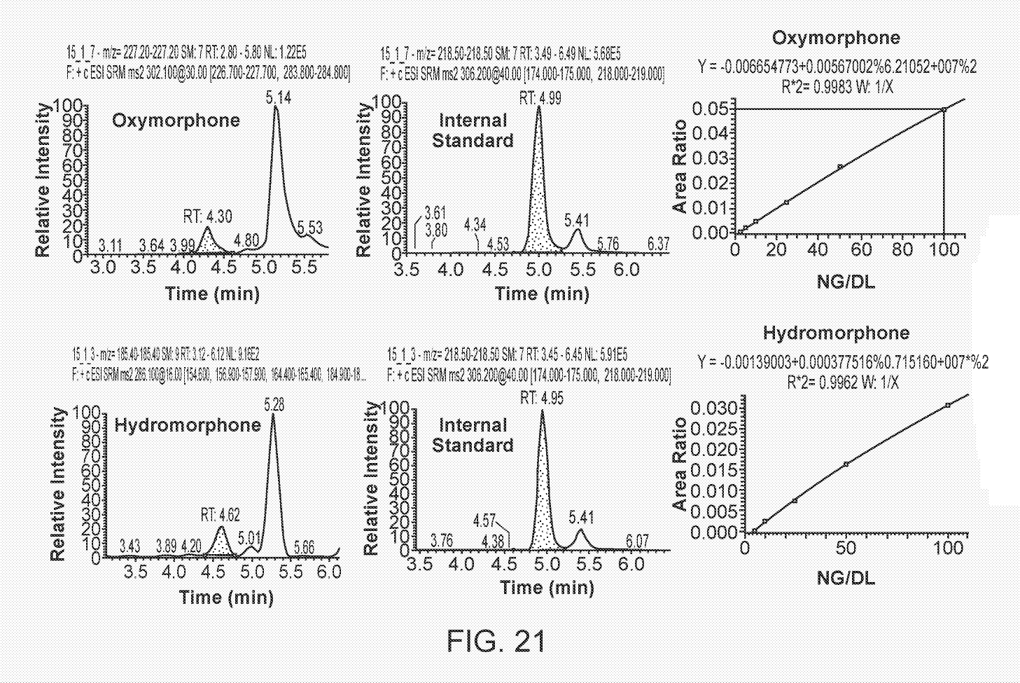

FIG. 20 shows chromatogram of letrozole, exemestane, and anastrozole.

### Example 3

### Opiates

In this assay, whole blood was centrifuged and spiked with opiate standards at different concentration levels to serve as assay calibrators.

10 uL and 15 uL MITRA® tips were dipped into the whole blood calibrators until fully saturated. Tips saturated with whole blood calibrators were left to dry at room temperature for at least 2.5 hours.

400 uL of extraction buffer (deuterated opiate internal standards in 65% ethyl acetate: 0.1% formic acid in methanol) was used to extract opiates from tips on a vortex for 40 minutes. Alternatively 500 uL of 60:40 ethyl acetate and methanol with 1% formic acid was used to extract opiates from tips on a vortex for 1 hour at 850 rpm. The tips were then discarded and the extracted samples were dried down completely under 60° C. nitrogen gas by the Porvair. The samples were then resuspended in 30% MeOH:0.1% FA in water, vortexed. Alternatively, samples were resuspended in 230 uL of 50:50 methanol and water with 0.1% formic acid. Samples were then injected into the LC-MS/MS for quantitation on ESI positive mode on a Thermo Ultra triple quadropole mass spectrometer. For mobile phase A, 0.1% formic acid in water was used. For mobile phase B, 100% acetonitrile was used. Agilent phenyl hexyl 3×100 mm column was used. The run time was 9 minutes.

Table 4 shows the linear range of each opiate in 10 uL tip vs. 15 uL tip.

| | 10 uL Tip | 15 uL Tip |
|---|---|---|
| ng/mL | range | range |
| Codeine | 5-1000 | 2.5-1000 |
| Noroxycodone | 10-1000 | 5-1000 |
| Hydrocodone | 5-1000 | 5-1000 |
| Morphine | 10-1000 | 5-1000 |
| Hydromorphone | 10-1000 | 10-1000 |
| Norhydrocodone | 5-1000 | 5-1000 |
| Oxymorphone | 5-1000 | 5-1000 |
| 6MAM | 10-1000 | 10-1000 |
| Oxycodone | 10-1000 | 5-1000 |
| Fentanyl | 2.5-1000 | 1-1000 |
| Norfentanyl | 2.5-1000 | 1-1000 |

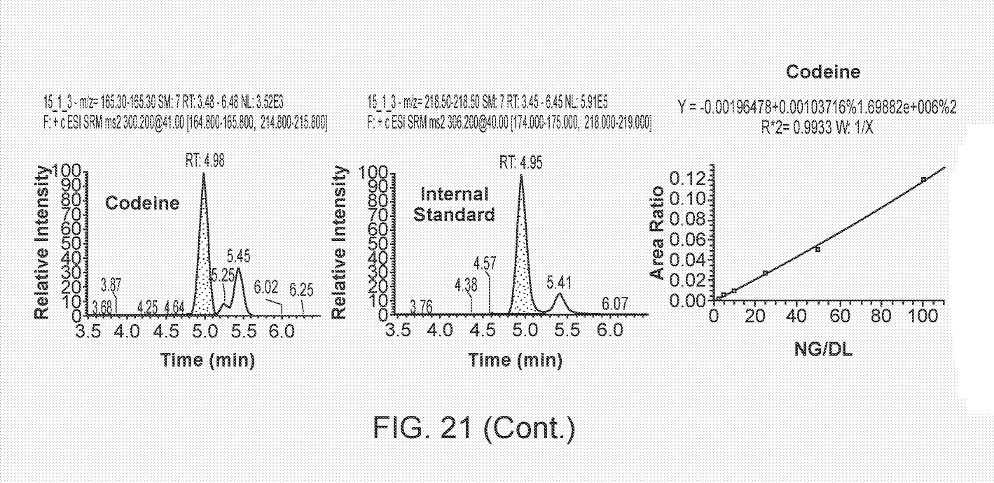

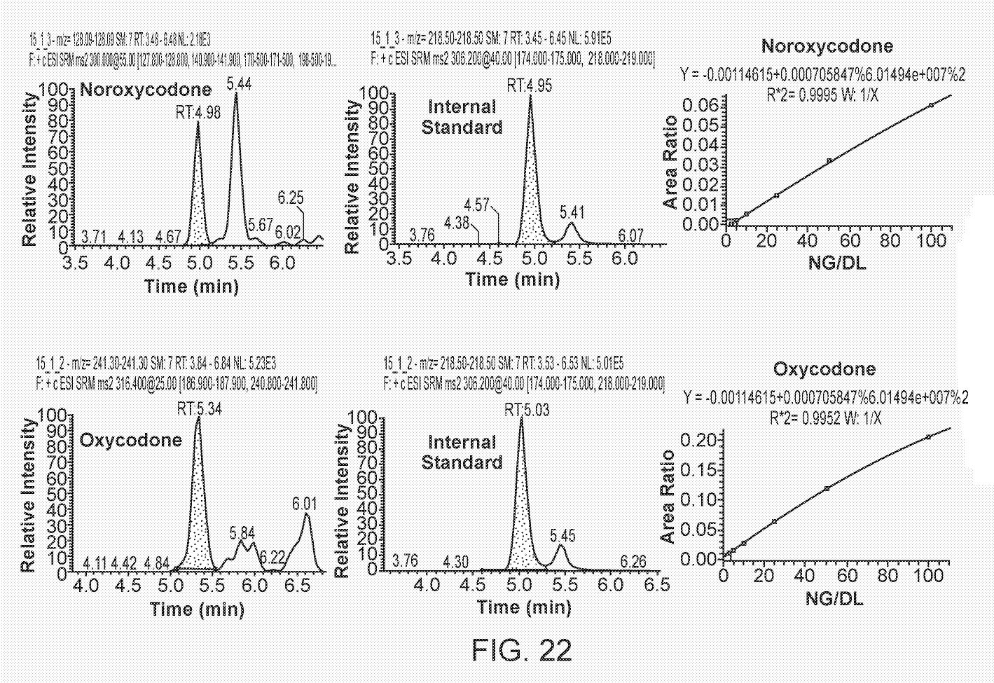

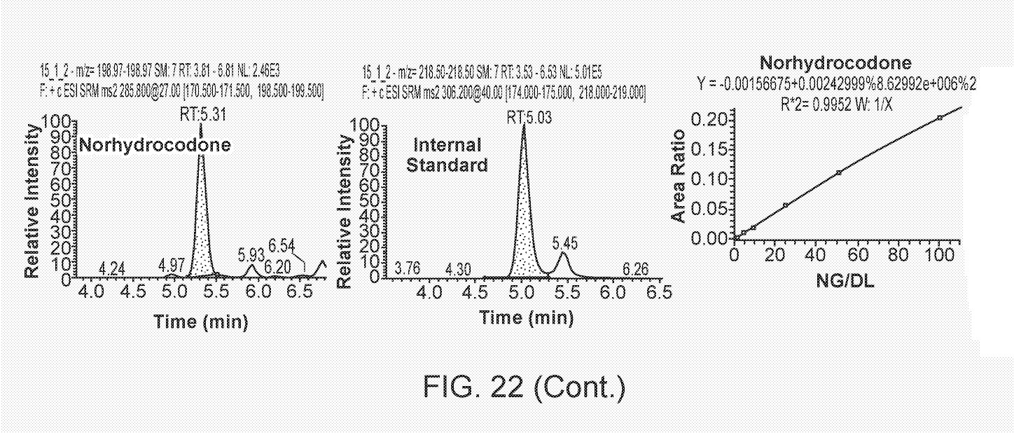

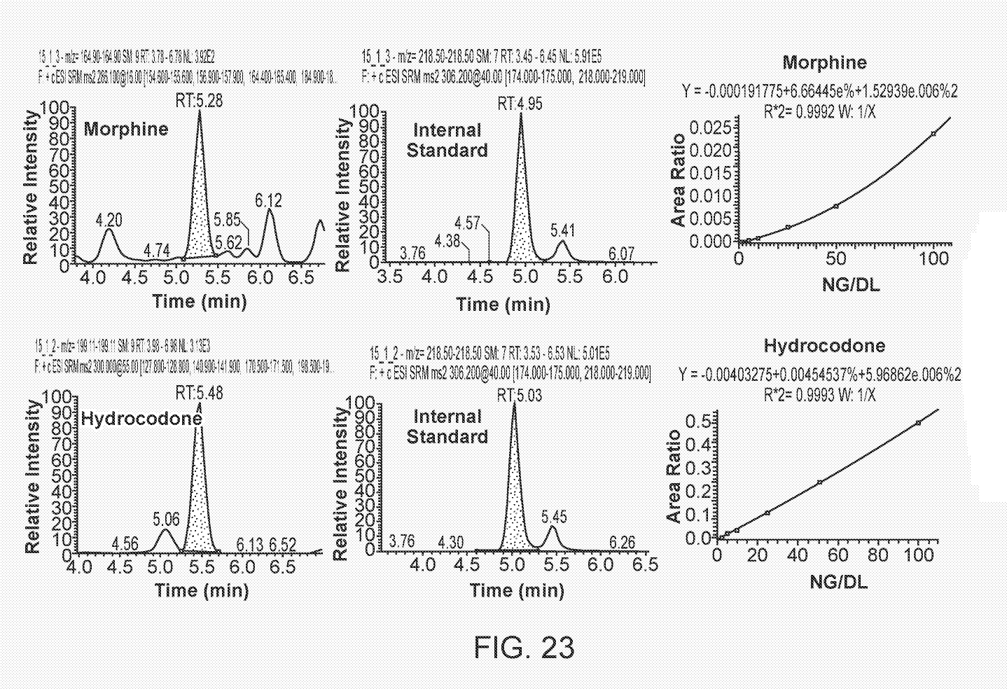

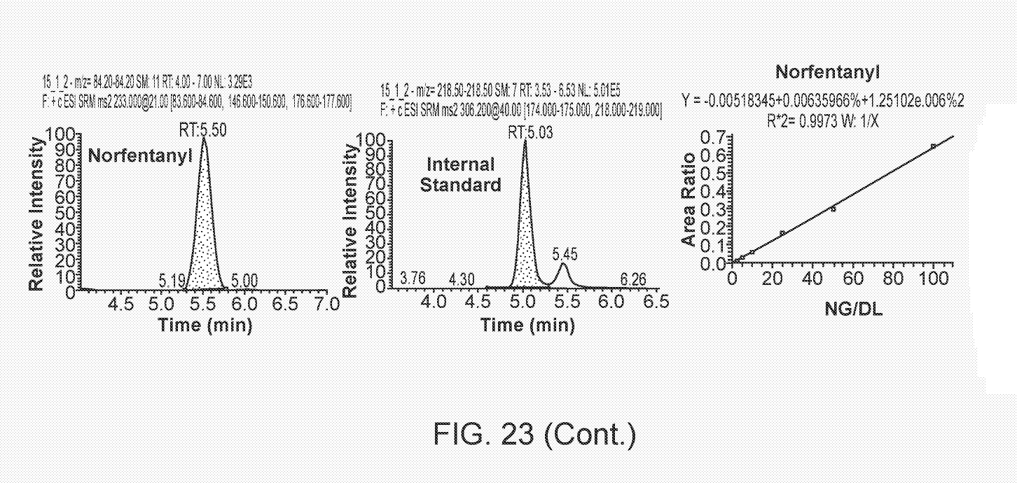

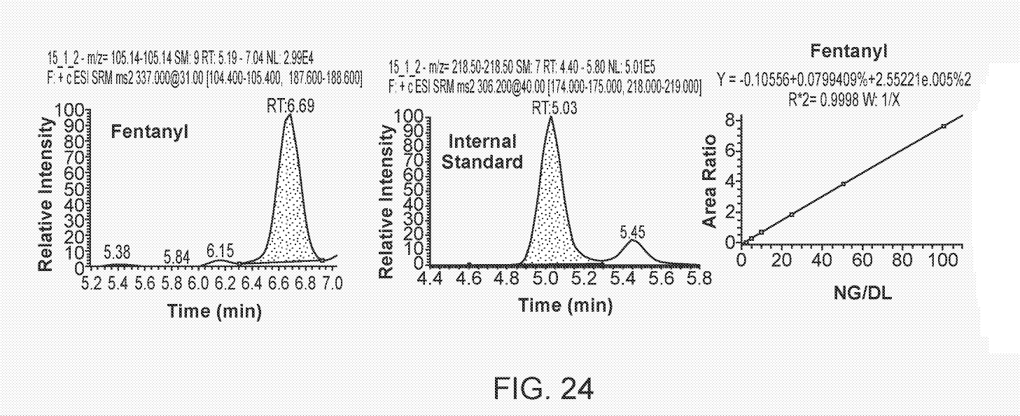

FIGS. 21 to 24 show exemplary chromatogram of opiates and corresponding internal standard.

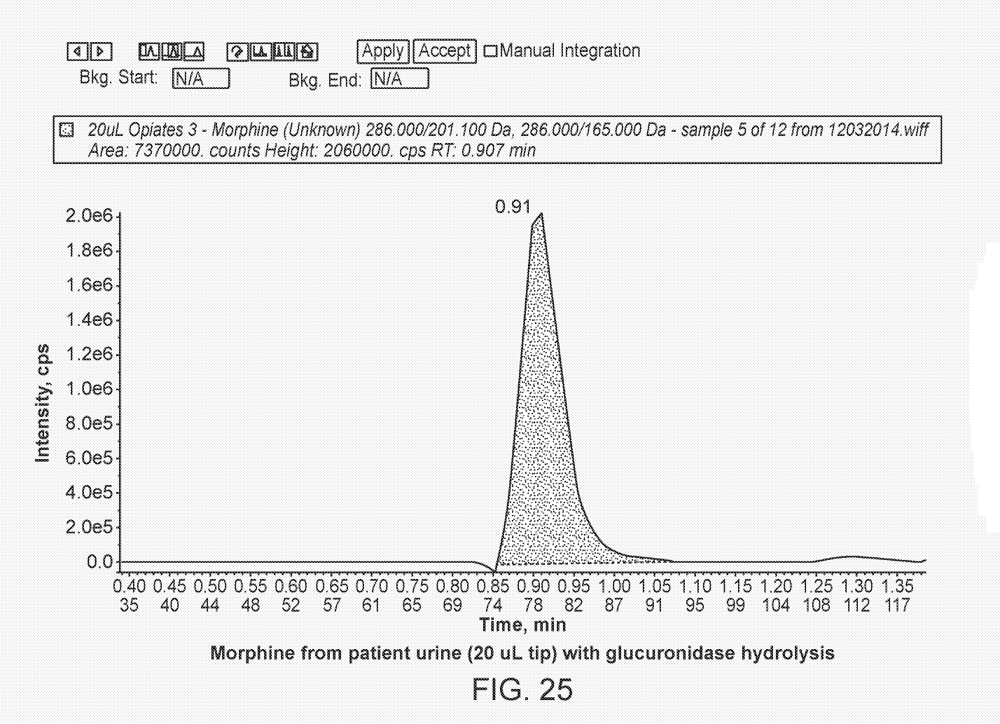

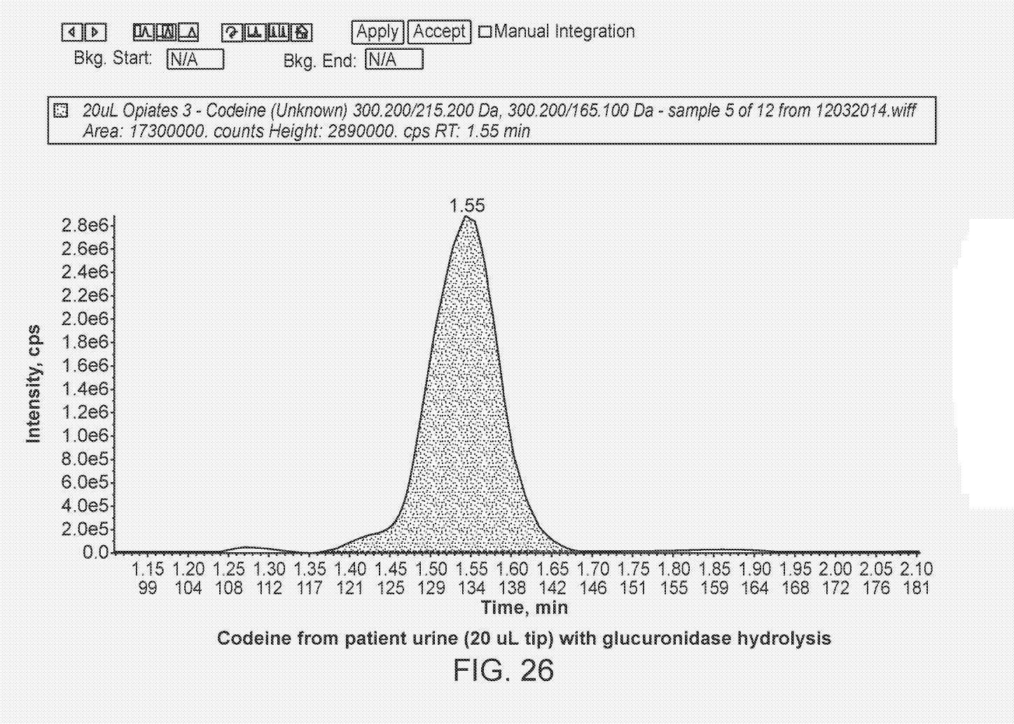

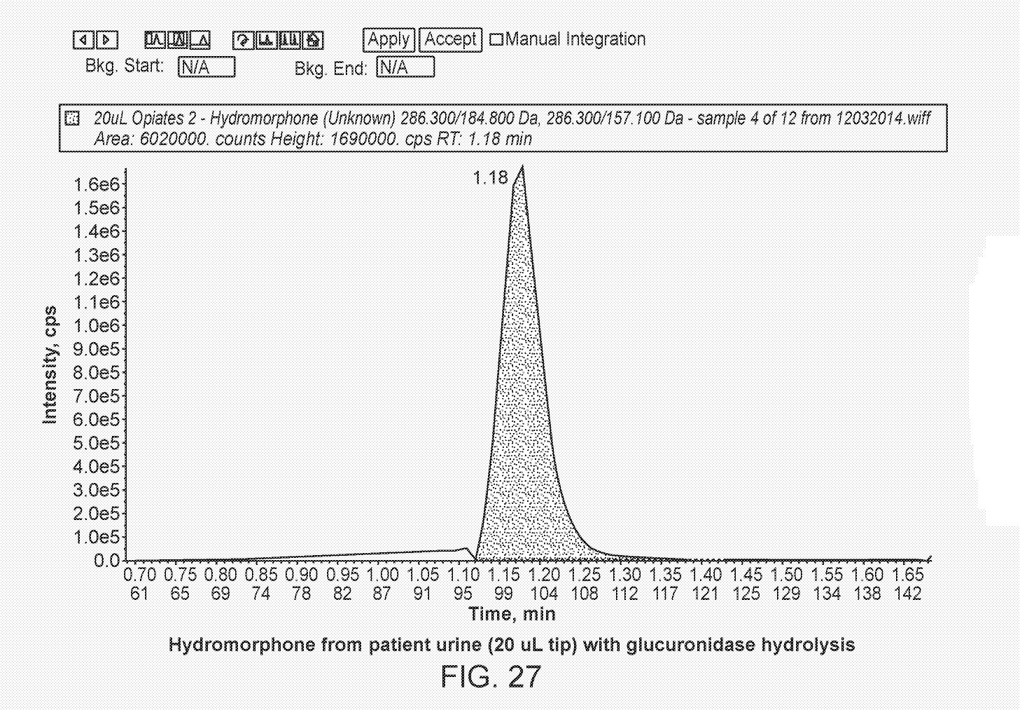

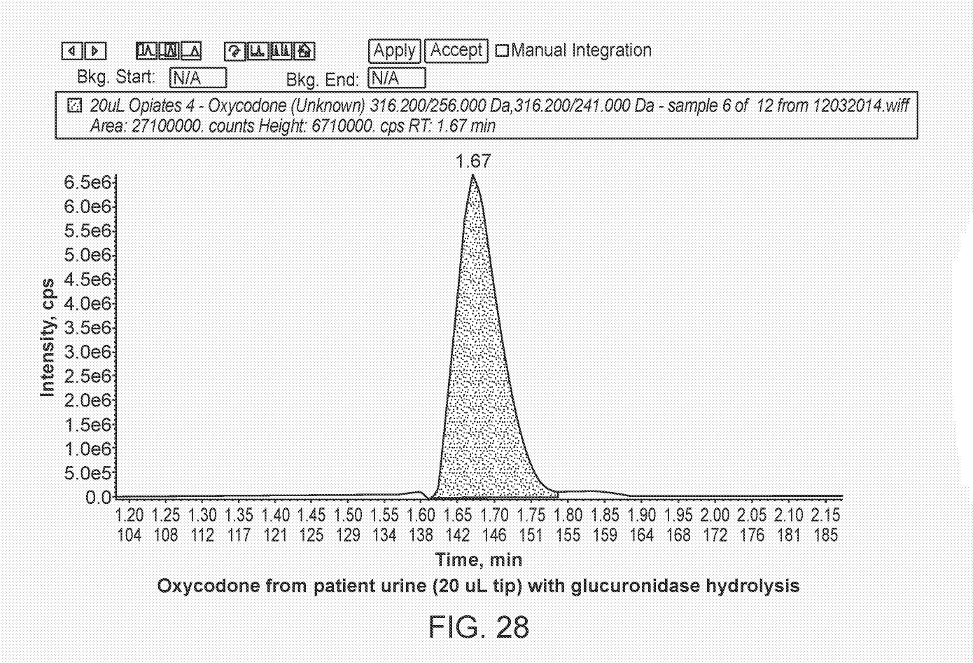

FIGS. 25 to 28 show morphine, codeine, hydromorphone, and oxycodone (respectively) data obtained from patient urine using 20 uL MITRA® tip with glucuronidase hydrolysis.

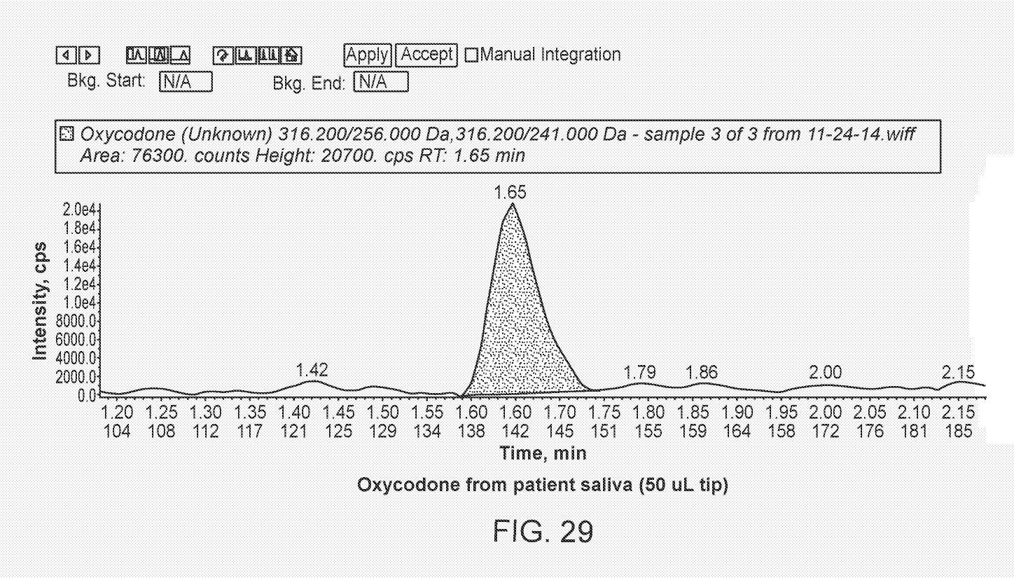

FIG. 29 shows oxycodone data obtained from patient saliva using 50 uL MITRA® tip.

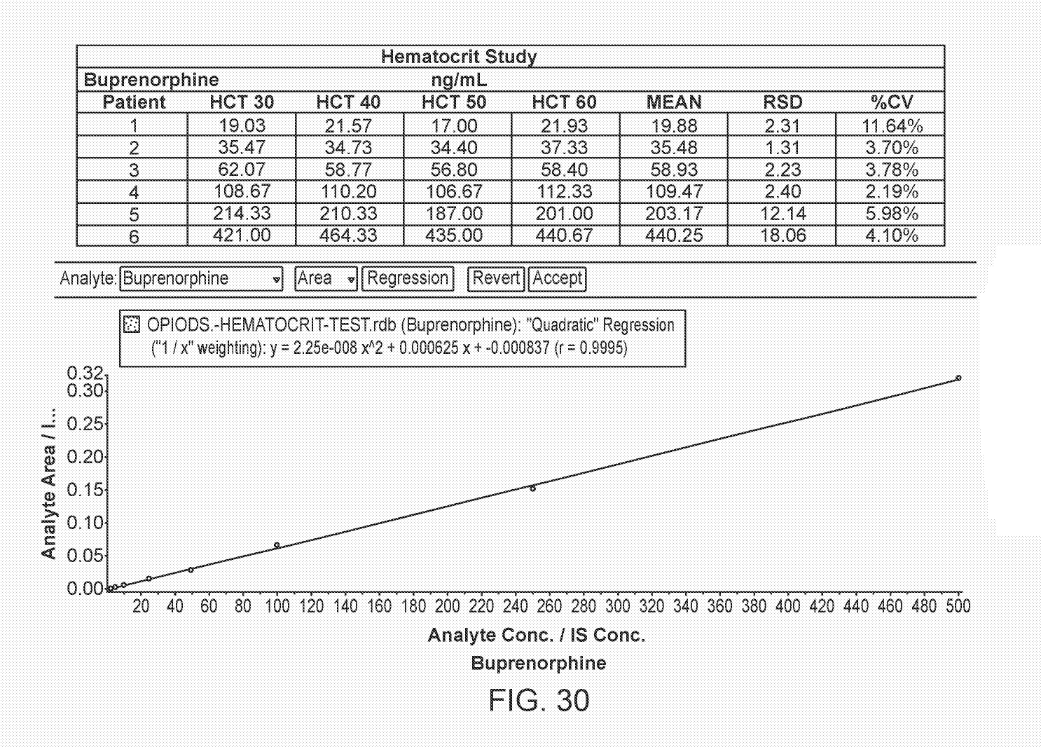

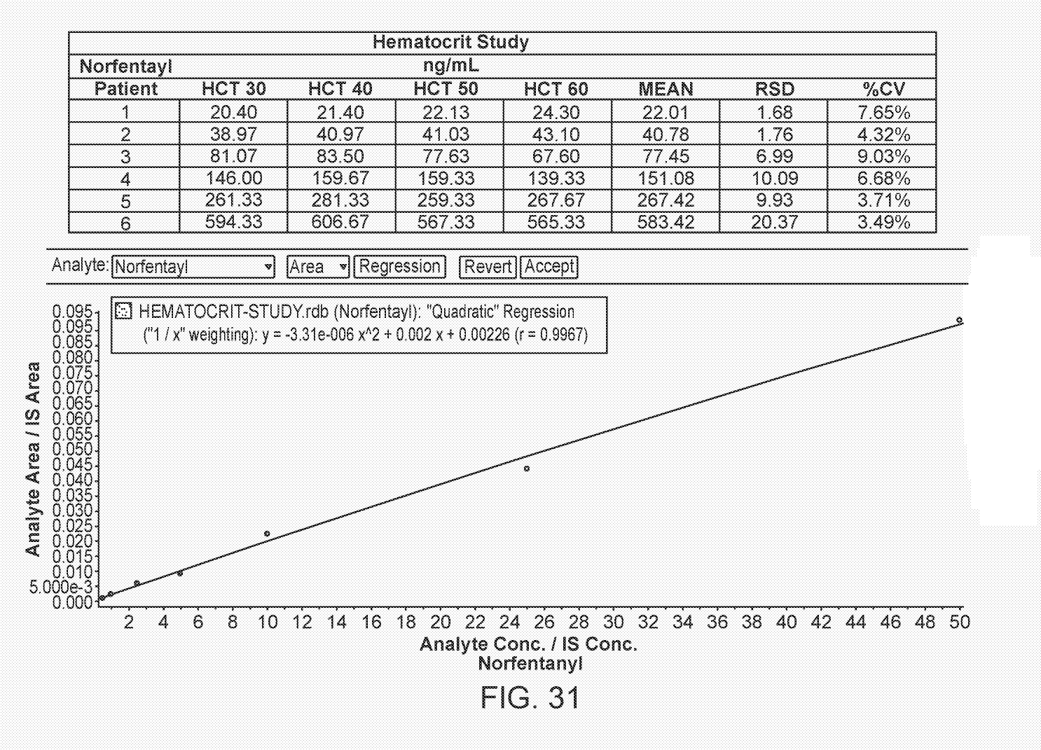

FIGS. 30 and 31 show the results of hematocrit study of buprenorphine and norfentanyl, respectively.

### Example 4

### Benzodiazepines

In this assay, whole blood was centrifuged and spiked with benzodiazepine standards at different concentration levels to serve as assay calibrators.

10 uL and 20 uL MITRA® tips were dipped into the whole blood calibrators until fully saturated. Tips saturated with whole blood calibrators were left to dry at room temperature for at least 2.5 hours.

400 uL of extraction buffer (deuterated benzodiazepine internal standards in 65% ethyl acetate: 0.1% formic acid in methanol) was used to extract opiates from tips on a vortex for 40 minutes. Alternatively 500 uL of 60:40 ethyl acetate and methanol with 1% formic acid (or alternatively, 0.1% formic acid) was used to extract benzodiazepines from tips on a vortex for 1 hour at 850 rpm. The tips were then discarded and the extracted samples were dried down completely under 60° C. nitrogen gas by the Porvair. The samples were then resuspended in 30% MeOH:0.1% formic acid in water, vortexed. Alternatively, samples were resuspended in 230 uL of 50:50 methanol and water with 0.1% formic acid. Alternatively, samples were resuspended in 200 uL of 0.1% formic acid in 10% methanol and 90% water. Samples were vortexed at 1200 rpm for 5 to 30 minutes. Samples were then injected into the LC-MS/MS for quantitation on ESI positive mode on a Thermo Ultra triple quadropole mass spectrometer. For mobile phase A, 0.1% formic acid in water was used. Alternatively, 20 mM ammonium acetate at pH 5.2 was used. For mobile phase B, 100% acetonitrile was used. Agilent phenyl hexyl 3×100 mm column was used. Alternatively, BDS Hypersil C18, 100×3 mm, 3μ column was used. The run time was 6 minutes.

The flow rate of 0.7 mL/minute was obtained: 0-60 sec-90% A: 10% B; 60-210 sec-ramp to 30% B; 210-360 sec-ramp to 65% B; 360-420 sec-ramp to 100% B; 420-480 sec-step 100% B; 480-600 sec-step 90% A: 10% B.

Table 5 shows benzodiazepines analyzed on 20 uL tips.

| | Parent | Products | Tube Lens | Collison Energy | Retention Time | LOQ on Mitra Tip-ng/mL |
|---|---|---|---|---|---|---|
| Bromazepam | 316 | 214 | 101 | 28 | 3.23 | 5 |
| | 316 | 270 | 101 | 35 | | |
| Oxazepam | 287 | 104 | 102 | 35 | 3.68 | 2.5 |
| | 287 | 241 | 102 | 22 | | |
| Clobazam | 300 | 224 | 102 | 21 | 3.75 | 1 |
| | 300 | 259 | 102 | 21 | | |
| Nitrazepam | 282 | 180 | 100 | 35 | 3.74 | 1 |
| | 282 | 236 | 100 | 26 | | |
| Alprazolam | 309.1 | 165 | 124 | 30 | 3.74 | 5 |
| | 309.1 | 280.9 | 124 | 26 | | |
| Triazolam | 343 | 206 | 107 | 19 | 3.8 | 1 |
| | 343 | 308 | 107 | 26 | | |
| Clonazepam | 316 | 214 | 101 | 28 | 3.82 | 2.5 |
| | 316 | 270 | 101 | 25 | | |
| Flurazepam | 388 | 287.9 | 127 | 24 | 3.82 | 0.5 |
| | 388 | 315 | 127 | 24 | | |
| Lorazepam | 321 | 229.1 | 104 | 24 | 3.79 | 1 |
| | 321 | 331 | 104 | 23 | | |
| Flunitrazepam | 314 | 211 | 102 | 35 | 4.02 | 0.5 |
| | 314 | 268 | 102 | 26 | | |
| Temazepam | 301.1 | 177 | 92 | 25 | 4.08 | 0.5 |
| | 301.1 | 255 | 92 | 19 | | |
| Midazolam | 326 | 129 | 116 | 29 | 4.2 | 10 |
| | 326 | 244 | 116 | 25 | | |
| Nordiazepam | 271 | 139.8 | 101 | 27 | 4.1 | 5 |
| | 271 | 165 | 101 | 28 | | |
| Phenazepam | 351 | 185.9 | 127 | 38 | 4.23 | 5 |
| | 351 | 206 | 127 | 34 | | |
| Chlordiazepam | 301 | 259 | 107 | 23 | 4.1 | 0.5 |
| | 301 | 224 | 107 | 19 | | |
| Diazepam | 285 | 154 | 99 | 25 | 4.5 | 1 |
| | 285 | 193 | 99 | 32 | | |
| Prazepam | 325 | 165 | 107 | 38 | 5.03 | 0.5 |
| | 325 | 271 | 107 | 21 | | |
| Medazepam | 271 | 180 | 98 | 25 | 5.15 | 1 |
| | 271 | 207.1 | 98 | 29 | | |

Table 6 shows the linear range of each opiate in 10 uL tip vs. 20 uL tip.

| | 10 uL Tip | 20 uL Tip | | | Therapeutic |
|---|---|---|---|---|---|
| | Range | Range | | | Range |
| | (ng/mL) | (ng/mL) | Mass | RT | (ng/mL) |
| Bromazepam | 10-1000 | 5-1000 | 316 | 3.23 | 10-250 |
| Oxazepam | 5-1000 | 2.5-1000 | 287 | 3.68 | 200-1400 |
| Clobazam | 2.5-1000 | 1-1000 | 300 | 3.75 | |
| Nitrazepam | 2.5-1000 | 1-1000 | 282 | 3.74 | |
| Alprazolam | 10-1000 | 5-1000 | 308 | 3.74 | 10-50 |
| Triazolam | 2.5-1000 | 1-1000 | 344 | 3.8 | |
| Clonazepam | 5-1000 | 2.5-1000 | 316 | 3.82 | 10-100 |
| Flurazepam | 1-1000 | 0.5-1000 | 388 | 3.82 | |
| Lorazepam | 2.5-1000 | 1-1000 | 321 | 3.79 | 5-100 |
| Flunitrazepam | 1-1000 | 0.5-1000 | 314 | 4.02 | 5-50 |
| Temazepam | 1-1000 | 0.5-1000 | 300 | 4.08 | |
| Midolazam | 25-1000 | 10-1000 | 326 | 4.2 | |
| Nordiazepam | 10-1000 | 5-1000 | 271 | 4.1 | 200-1000 |
| Phenazepam | 10-1000 | 5-1000 | 349 | 4.23 | |
| Chlorodiazepam | 1-1000 | 0.5-1000 | 301 | 4.1 | 10-100 |
| Diazepam | 2.5-1000 | 1-1000 | 285 | 4.5 | 100-800 |
| Prazepam | 1-1000 | 0.5-1000 | 325 | 5.03 | |
| Medazepam | 2.5-1000 | 1-1000 | 271 | 5.15 | |

### Example 5

### Barbiturates

In this assay, urine samples negative for barbiturates were spiked with barbiturate standards at different concentration levels to serve as assay calibrators.

20 uL MITRA® tips were dipped into the urine calibrators until fully saturated. Tips saturated with urine calibrators were left to dry at room temperature.

Samples were extracted in methanol for 1 hour. Extracted samples were hydrolyzed for 1 hour at 60° C. on thermomixer. Samples were then centrifuged and supernatant was injected into the LC-MS/MS for quantitation. Liquid chromatography run time was 5.75 minutes. Acquisition window was 2.5 minutes. The assay allowed for 2 plex, data every 2.75 minutes. 0.03% NH₄OH was used for mobile phase A. 90% CAN and 10% MP A was used for mobile phase B.

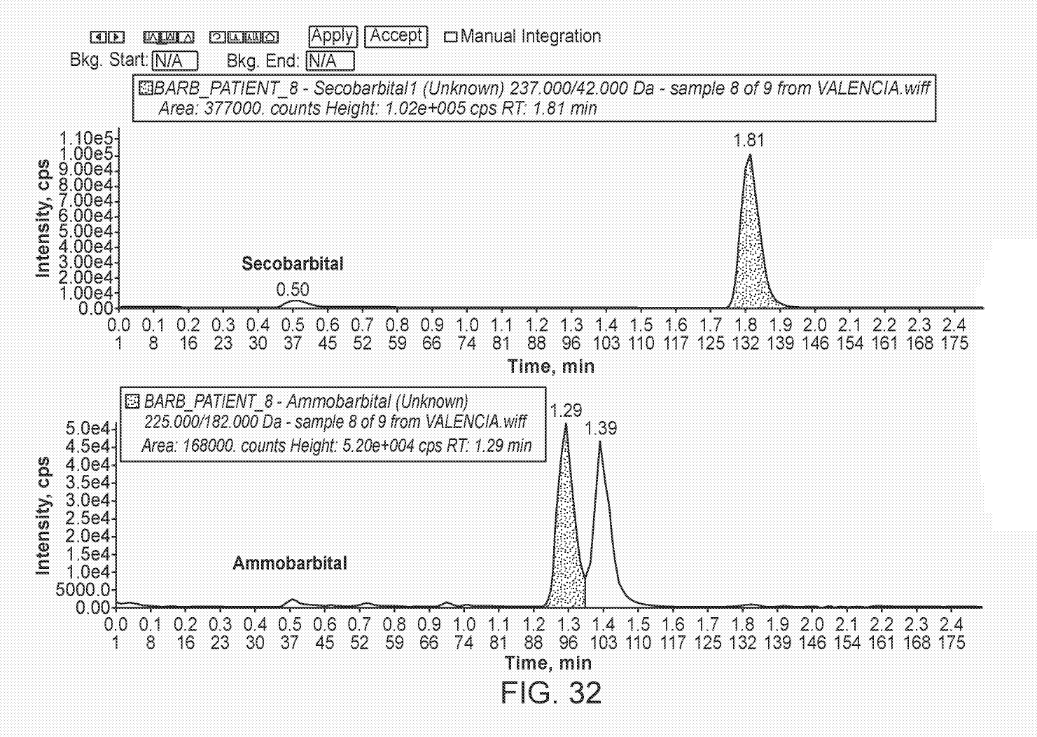

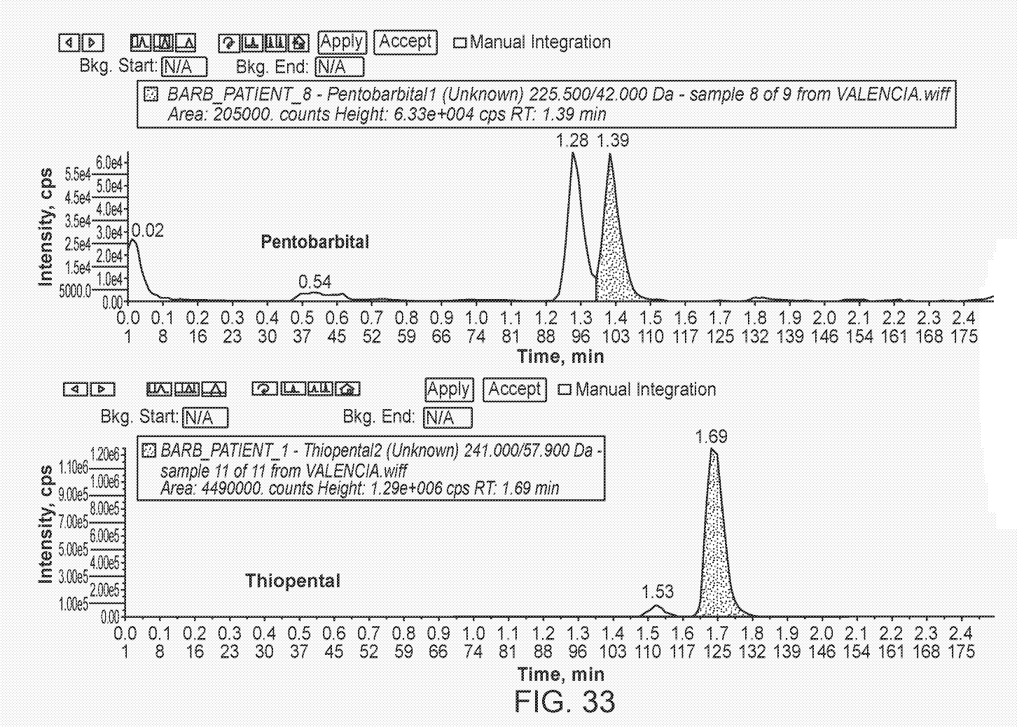

FIGS. 32 and 33 show the results of negative urine spiked with barbiturates (secobarbital, ammobarbital, pentobarbital, and thiopental).

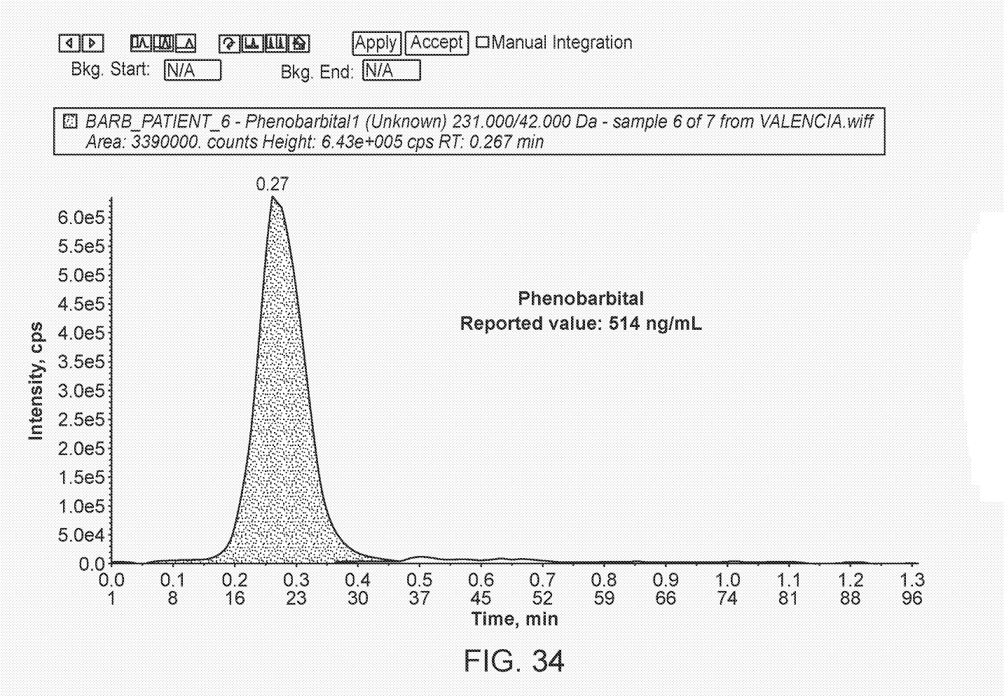

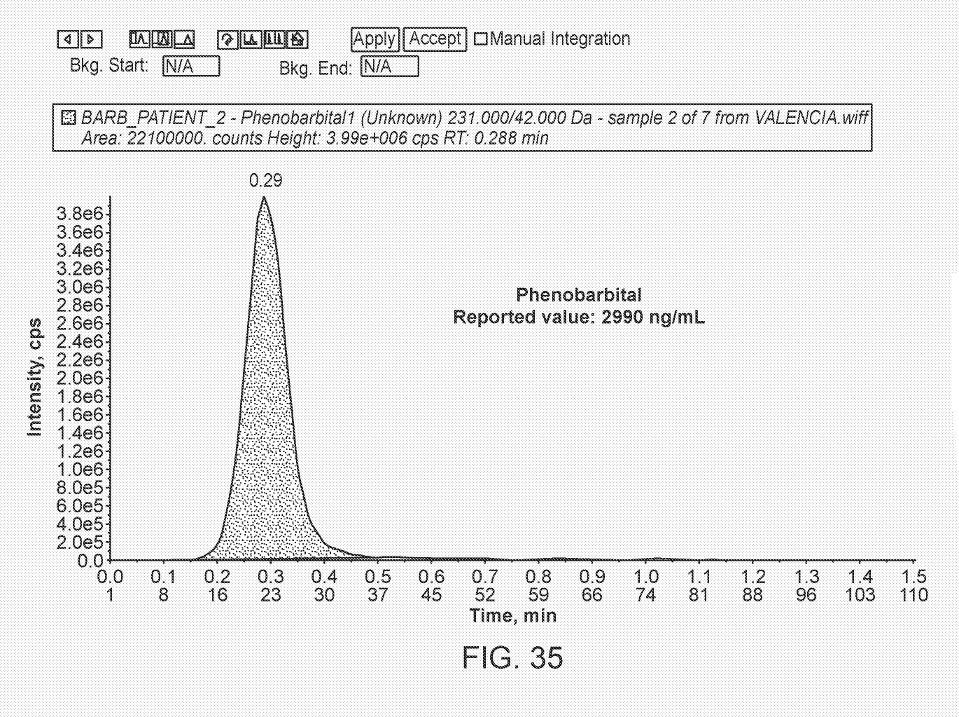

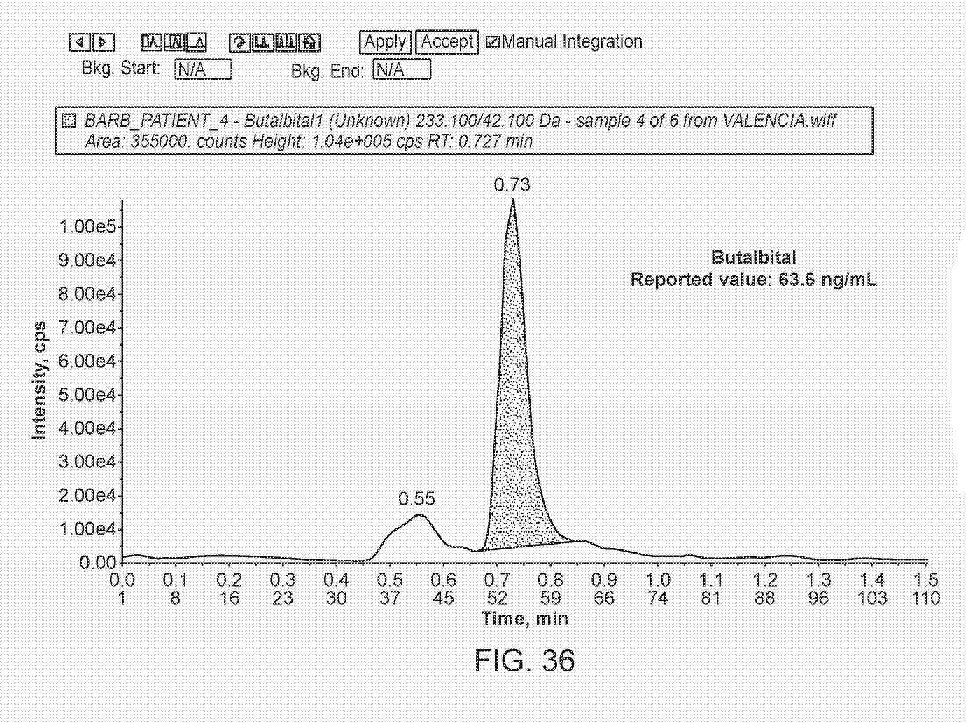

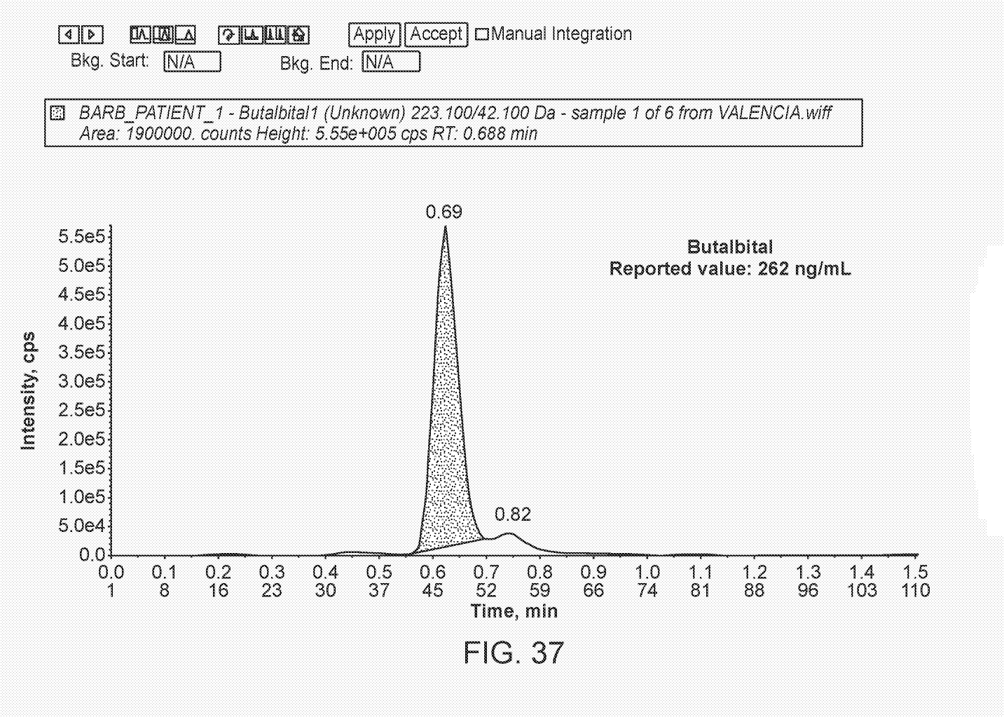

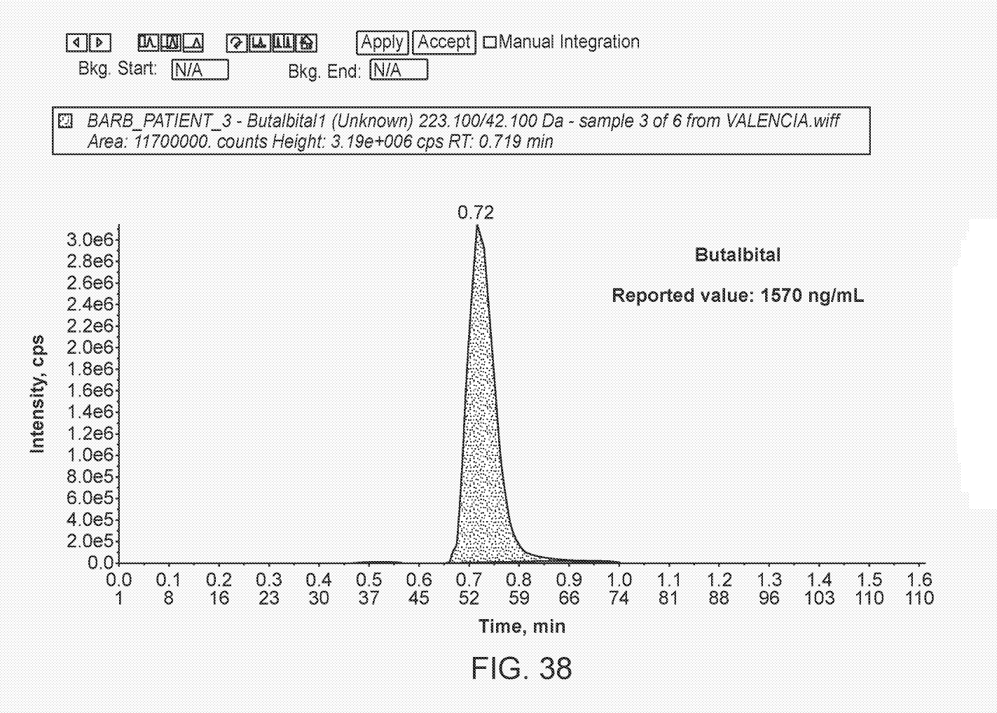

FIGS. 34 to 38 show the results of various patient samples quantitated for phenobarbital and butalbital.

### Example 6

### THC

In this assay, patient urine samples were analyzed.

20 uL MITRA® tips were dipped into the urine samples until fully saturated. Tips saturated with urine samples were left to dry at room temperature.

Samples were extracted in 100% methanol by vortexing at 900 rpm for 1 hour. Samples were dried down with nitrogen air at 60° C. until completely dry. Samples were resuspended in 200 uL of 20 mM sodium citrate buffer at pH 4.5. Glucuronidase was added to the sample and incubated on thermomixer for 40 minutes at 60° C. Samples were centrifuged at 5500 rpm for 3 minutes and supernatant was injected into the LC-MS/MS (ABI5500) for quantitation.

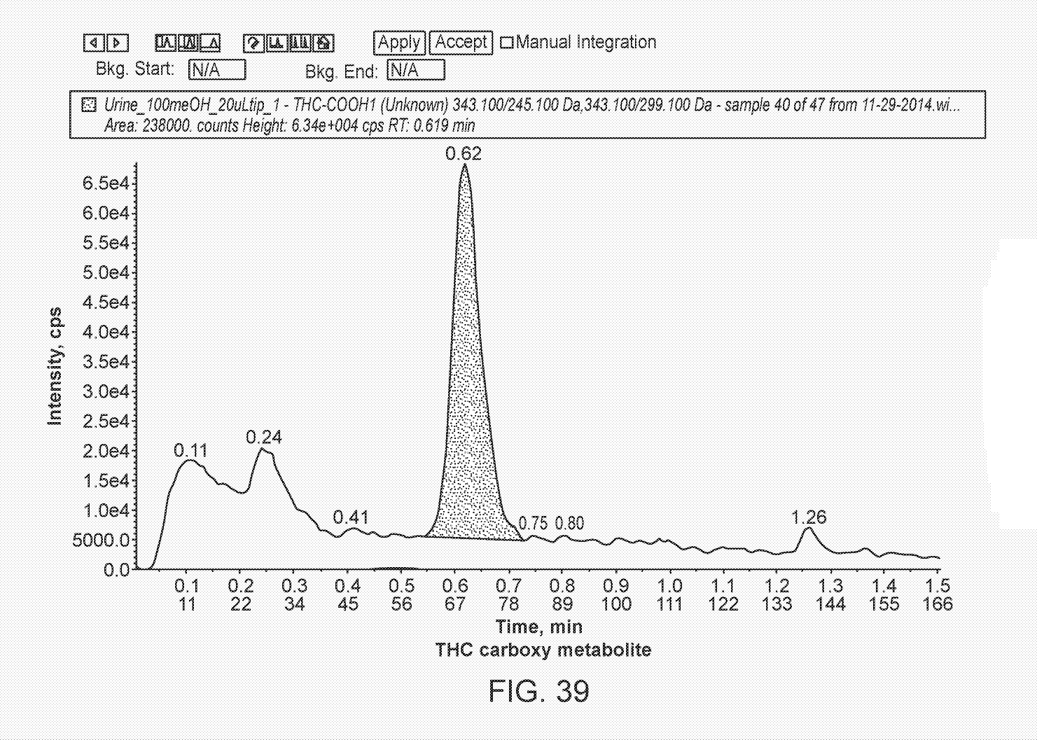

FIG. 39 shows the results of THC carboxy metabolite analysis in patient sample using 20 uL tip and glucuronidase hydrolysis.

### Example 7

### Anti-Epileptic Drugs

In this assay, patient whole blood samples were analyzed.

20 uL MITRA® tips were dipped into the whole blood samples until fully saturated. Tips saturated with whole blood samples were left to dry at room temperature.

Samples were extracted in 90% methanol and 10% water for 1 hour. Samples were dried down with nitrogen air at 60° C. until completely dry. Samples were resuspended in 0.1% formic acid in water and was injected into the LC-MS/MS for quantitation. 5 uL was injected into the Thermo Fisher Quantiva. Thermo Fisher Beta-Basic C18, 100×3 mm analytical column was used. Mobile Phase A: 0.1% FA; Mobile Phase B: Methanol.

Table 7 shows mass transitions used in the mass spectrometric analysis.

| Compound | Start Time (min) | End Time (min) | Polarity | Precursor (m/z) | Product (m/z) | Collision Energy (V) |
|---|---|---|---|---|---|---|
| Felbamate | 0 | 6.5 | Positive | 117 | 91 | 24 |
| Felbamate Q | 0 | 6.5 | Positive | 117 | 115 | 20 |
| ethosuximide Q | 0 | 6.5 | Positive | 142.2 | 39.3 | 39 |
| ethosuximide | 0 | 6.5 | Positive | 142.2 | 44.3 | 32 |
| Pregabalin | 0 | 6.5 | Positive | 160.1 | 55.25 | 23 |
| Pregabalin Q | 0 | 6.5 | Positive | 160.1 | 77.2 | 35 |
| Pregabalin D6 | 0 | 6.5 | Positive | 166.2 | 148 | 9 |
| Levetiracetam Q | 0 | 6.5 | Positive | 171.2 | 69.2 | 29 |
| Levetiracetam | 0 | 6.5 | Positive | 171.2 | 126.2 | 16 |
| Gabapentin Q | 0 | 6.5 | Positive | 172.3 | 67.2 | 30 |
| Gabapentin | 0 | 6.5 | Positive | 172.3 | 91.2 | 26 |
| Levetiracetam D6 | 0 | 6.5 | Positive | 177 | 132 | 16 |
| Gabapentin D10 | 0 | 6.5 | Positive | 182.2 | 147 | 26 |
| Gabapentin D10 | 0 | 6.5 | Positive | 182.2 | 164 | 26 |
| Zonisamide | 0 | 6.5 | Positive | 213.2 | 77.2 | 32 |
| Zonisamide Q | 0 | 6.5 | Positive | 213.2 | 102.1 | 30 |
| Zonisamide 13C6 | 0 | 6.5 | Positive | 219 | 82 | 31 |
| Zonisamide 13C6 | 0 | 6.5 | Positive | 219 | 108 | 30 |
| Carbamazepine Q | 0 | 6.5 | Positive | 237 | 179 | 36 |
| Carbamazepine | 0 | 6.5 | Positive | 237 | 194.1 | 20 |
| Rufinamide | 0 | 6.5 | Positive | 239 | 127.2 | 28 |
| Rufinamide Q | 0 | 6.5 | Positive | 239 | 261 | 10 |
| Carbamazepine D10 | 0 | 6.5 | Positive | 247 | 204 | 30 |
| lacosamide Q | 0 | 6.5 | Positive | 251 | 65.2 | 58 |
| lacosamide | 0 | 6.5 | Positive | 251 | 91.2 | 23 |
| 1012 Epoxide Q | 0 | 6.5 | Positive | 253 | 167.2 | 39 |
| 1011 Epoxide | 0 | 6.5 | Positive | 253 | 180.1 | 28 |
| Phenytoin | 0 | 6.5 | Positive | 253.1 | 104.2 | 34 |
| Phenytoin Q | 0 | 6.5 | Positive | 253.1 | 182.2 | 19 |
| Lacosamide 13C D3 | 0 | 6.5 | Positive | 255.1 | 91.1 | 23 |
| lamotrigine Q | 0 | 6.5 | Positive | 256 | 145 | 39 |
| lamotrigine | 0 | 6.5 | Positive | 256 | 211 | 27 |
| 1011 Epoxide 13C6 | 0 | 6.5 | Positive | 259.2 | 186.2 | 30 |
| Lamotrigine 13C 15N4 | 0 | 6.5 | Positive | 261 | 213 | 27 |
| Phenytoin D10 | 0 | 6.5 | Positive | 263 | 192 | 19 |
| Eslicarbazepine Q | 0 | 6.5 | Positive | 297.1 | 179 | 44 |
| Eslicarbazepine | 0 | 6.5 | Positive | 297.1 | 194 | 58 |
| Felbamate Q | 0 | 6.5 | Positive | 339 | 117.3 | 21 |
| Felbamate | 0 | 6.5 | Positive | 339 | 261 | 9 |
| Tiagabine | 0 | 6.5 | Positive | 376.2 | 111.1 | 33 |
| Tiagabine Q | 0 | 6.5 | Positive | 376.2 | 149.1 | 27 |
| Tiagabine D6 | 0 | 6.5 | Positive | 382 | 253.1 | 25 |

Table 8 shows the calibration standards used in the analysis.

Calibration Standards (ug/mL)

| Standard | Ethosuximide | Gabapentin | Levetiracetam | Pregabalain | Zonisamide | Lamotrigine | Lacosamdie |
|---|---|---|---|---|---|---|---|
| 1 | 5 | 1.25 | 2.5 | 0.5 | 2.5 | 1.25 | 1 |
| 2 | 10 | 2.5 | 5 | 1 | 5 | 2.5 | 2.5 |
| 3 | 25 | 6.25 | 12.5 | 2.5 | 12.5 | 6.25 | 5 |
| 4 | 40 | 10 | 20 | 4 | 20 | 10 | 8 |
| 5 | 50 | 12.5 | 25 | 5 | 25 | 12.5 | 10 |
| 6 | 75 | 18.75 | 37.5 | 7.5 | 37.5 | 18.75 | 15 |
| 7 | 100 | 25 | 50 | 10 | 50 | 25 | 20 |

| Standard | Rufinamide | Felbamate | 10, 11 carbamazepine epoxide | Phenytoin | Carbamazepine | Eslicarbazepine | Tiagabine |
|---|---|---|---|---|---|---|---|
| 1 | 2.5 | 2.5 | 1.25 | 1 | 1 | 2.5 | 0.01 |
| 2 | 5 | 5 | 2.5 | 2.5 | 2.5 | 5 | 0.02 |
| 3 | 12.5 | 12.5 | 6.25 | 5 | 5 | 12.5 | 0.05 |
| 4 | 20 | 20 | 10 | 8 | 8 | 20 | 0.08 |
| 5 | 25 | 25 | 12.5 | 10 | 10 | 25 | 0.1 |
| 6 | 37.5 | 37.5 | 18.75 | 15 | 15 | 37.5 | 0.15 |
| 7 | 50 | 50 | 25 | 20 | 20 | 50 | 0.2 |

Within run precision: Acceptability criteria: The % CV should be less than allowable ≦TEa/2. The Tea for this assay is determined to be 30%. Ten replicates of each quality control were analyzed within a single assay in the following order; low, medium and high.

Table 9 shows the within run precision of Ethosuximide. The % CV for Ethosuximide ranged from 5.16% to 2.23% across all three quality control levels.

ETHOSUXIMIDE

| Low QC | 15 ug/mL | Medium QC | 30 ug/mL | High QC | 60 ug/mL |
|---|---|---|---|---|---|
| Run 1 | 17.65 | Run 1 | 33.14 | Run 1 | 63.12 |
| Run 2 | 15.46 | Run 2 | 31.03 | Run 2 | 60.12 |
| Run 3 | 15.34 | Run 3 | 32.09 | Run 3 | 59.87 |
| Run 4 | 15.16 | Run 4 | 30.85 | Run 4 | 60.45 |
| Run 5 | 15.85 | Run 5 | 29.94 | Run 5 | 61.36 |
| Run 6 | 15.60 | Run 6 | 35.30 | Run 6 | 61.76 |
| Run 7 | 16.75 | Run 7 | 30.92 | Run 7 | 62.81 |
| Run 8 | 15.66 | Run 8 | 30.68 | Run 8 | 61.79 |
| Run 9 | 16.87 | Run 9 | 30.32 | Run 9 | 59.23 |
| Run 10 | 15.41 | Run 10 | 31.70 | Run 10 | 62.84 |
| MEAN | 15.97 | MEAN | 31.60 | MEAN | 61.34 |
| STDEV | 0.82 | STDEV | 1.60 | STDEV | 1.37 |
| % CV | 5.16% | % CV | 5.05% | % CV | 2.23% |
| % | 106.50% | % Accuracy | 105.32% | % | 102.23% |
| Accuracy | | | | Accuracy | |

Table 10 shows the within run precision of Gabapentin. The % CV for Gabapentin ranged from 7.01% to 3.61% across all three quality control levels.

GABAPENTIN

| | | Medium | | | |
|---|---|---|---|---|---|
| Low QC | 3.75 ug/mL | QC | 7.5 ug/mL | High QC | 15 ug/mL |
| Run 1 | 3.84 | Run 1 | 8.35 | Run 1 | 15.98 |
| Run 2 | 4.08 | Run 2 | 7.44 | Run 2 | 16.47 |
| Run 3 | 3.63 | Run 3 | 8.68 | Run 3 | 15.84 |
| Run 4 | 4.05 | Run 4 | 7.44 | Run 4 | 15.93 |
| Run 5 | 3.96 | Run 5 | 8.07 | Run 5 | 17.19 |
| Run 6 | 3.88 | Run 6 | 7.22 | Run 6 | 17.11 |
| Run 7 | 3.66 | Run 7 | 8.04 | Run 7 | 15.77 |
| Run 8 | 3.85 | Run 8 | 8.8 | Run 8 | 15.45 |
| Run 9 | 3.76 | Run 9 | 7.68 | Run 9 | 16.46 |
| Run 10 | 3.63 | Run 10 | 7.48 | Run 10 | 15.84 |
| MEAN | 3.83 | MEAN | 7.92 | MEAN | 16.20 |
| STDEV | 0.16 | STDEV | 0.56 | STDEV | 0.58 |
| % CV | 4.30% | % CV | 7.01% | % CV | 3.61% |
| % | 102.24% | % | 105.60% | % | 108.03% |
| Accuracy | | Accuracy | | Accuracy | |

Table 11 shows the within run precision of Levetiracetam. The % CV for Levetiracetam ranged from 8.46% to 4.17% across all three quality control levels.

LEVETIRACETAM

| Low QC | 7.5 ug/mL | Medium QC | 15 ug/mL | High QC | 30 ug/mL |
|---|---|---|---|---|---|
| Run 1 | 7.47 | Run 1 | 15.9 | Run 1 | 29.79 |
| Run 2 | 7.46 | Run 2 | 16.32 | Run 2 | 32.75 |
| Run 3 | 7.58 | Run 3 | 16.94 | Run 3 | 32.91 |
| Run 4 | 7.18 | Run 4 | 15.92 | Run 4 | 33.13 |
| Run 5 | 7.49 | Run 5 | 15.21 | Run 5 | 30.65 |
| Run 6 | 7.32 | Run 6 | 16.19 | Run 6 | 31.43 |
| Run 7 | 7.04 | Run 7 | 14.57 | Run 7 | 31.07 |
| Run 8 | 7.67 | Run 8 | 14.73 | Run 8 | 30.61 |
| Run 9 | 7.21 | Run 9 | 12.31 | Run 9 | 33.6 |
| Run 10 | 7.29 | Run 10 | 15.79 | Run 10 | 30.66 |
| MEAN | 7.37 | MEAN | 15.39 | MEAN | 31.66 |
| STDEV | 0.20 | STDEV | 1.30 | STDEV | 1.32 |
| % CV | 2.66% | % CV | 8.46% | % CV | 4.17% |
| % | 98.28% | % Accuracy | 102.59% | % | 105.53% |
| Accuracy | | | | Accuracy | |

Table 12 shows the within run precision of Pregabalin. The % CV for Pregabalin ranged from 6.10% to 4.08% across all three quality control levels.

PREGABALIN

| Low QC | 1.5 ug/mL | Medium QC | 3 ug/mL | High QC | 6 ug/mL |
|---|---|---|---|---|---|
| Run 1 | 1.57 | Run 1 | 3.34 | Run 1 | 6.1 |
| Run 2 | 1.52 | Run 2 | 3.67 | Run 2 | 6.27 |
| Run 3 | 1.55 | Run 3 | 3.23 | Run 3 | 6.45 |
| Run 4 | 1.49 | Run 4 | 3.07 | Run 4 | 6.89 |
| Run 5 | 1.53 | Run 5 | 3.15 | Run 5 | 6.68 |
| Run 6 | 1.52 | Run 6 | 3.14 | Run 6 | 6.97 |
| Run 7 | 1.66 | Run 7 | 3.34 | Run 7 | 6.21 |
| Run 8 | 1.53 | Run 8 | 3.37 | Run 8 | 6.87 |
| Run 9 | 1.41 | Run 9 | 3.5 | Run 9 | 6.3 |
| Run 10 | 1.54 | Run 10 | 3.03 | Run 10 | 6.02 |
| MEAN | 1.53 | MEAN | 3.28 | MEAN | 6.48 |
| STDEV | 0.06 | STDEV | 0.20 | STDEV | 0.35 |
| % CV | 4.08% | % CV | 6.10% | % CV | 5.41% |
| % | 102.13% | % Accuracy | 109.47% | % | 107.93% |
| Accuracy | | | | Accuracy | |

Table 13 shows the within run precision of Zonisamide. The % CV for Zonisamide ranged from 6.35% to 4.87% across all three quality control levels.

ZONISAMIDE

| Low QC | 7.5 ug/mL | Medium QC | 15 ug/mL | High QC | 30 ug/mL |
|---|---|---|---|---|---|
| Run 1 | 7.54 | Run 1 | 16.78 | Run 1 | 30.06 |
| Run 2 | 7.69 | Run 2 | 17.18 | Run 2 | 34.48 |
| Run 3 | 7.26 | Run 3 | 17.85 | Run 3 | 31.08 |
| Run 4 | 7.68 | Run 4 | 15.63 | Run 4 | 35.21 |
| Run 5 | 7.82 | Run 5 | 16.12 | Run 5 | 34.49 |
| Run 6 | 8.4 | Run 6 | 16.31 | Run 6 | 33.71 |
| Run 7 | 8.54 | Run 7 | 16.97 | Run 7 | 35.16 |
| Run 8 | 8.44 | Run 8 | 15.52 | Run 8 | 32.5 |
| Run 9 | 7.62 | Run 9 | 15.38 | Run 9 | 30.49 |
| Run 10 | 7.37 | Run 10 | 16.16 | Run 10 | 30.53 |
| MEAN | 7.84 | MEAN | 16.39 | MEAN | 32.77 |
| STDEV | 0.46 | STDEV | 0.80 | STDEV | 2.08 |
| % CV | 5.88% | % CV | 4.87% | % CV | 6.35% |
| % | 104.48% | % Accuracy | 109.27% | % | 109.24% |
| Accuracy | | | | Accuracy | |

Table 14 shows the within run precision of Lamotrigine. The % CV for Lamotrigine ranged from 6.77% to 6.10% across all three quality control levels.

LAMOTRIGINE

| | | Medium | | | |
|---|---|---|---|---|---|
| Low QC | 3.75 ug/mL | QC | 7.5 ug/mL | High QC | 15 ug/mL |
| Run 1 | 3.67 | Run 1 | 8.24 | Run 1 | 15.45 |
| Run 2 | 3.93 | Run 2 | 8.95 | Run 2 | 15.46 |
| Run 3 | 3.75 | Run 3 | 9.87 | Run 3 | 16.63 |
| Run 4 | 3.97 | Run 4 | 8.79 | Run 4 | 16.63 |
| Run 5 | 4.33 | Run 5 | 8.55 | Run 5 | 16.98 |
| Run 6 | 3.52 | Run 6 | 8.55 | Run 6 | 15.00 |
| Run 7 | 3.52 | Run 7 | 9.56 | Run 7 | 14.5 |
| Run 8 | 3.85 | Run 8 | 9.2 | Run 8 | 14.69 |
| Run 9 | 3.51 | Run 9 | 8.22 | Run 9 | 17.05 |
| Run 10 | 3.87 | Run 10 | 8.57 | Run 10 | 15.18 |
| MEAN | 3.79 | MEAN | 8.85 | MEAN | 15.76 |
| STDEV | 0.26 | STDEV | 0.55 | STDEV | 0.97 |
| % CV | 6.77% | % CV | 6.20% | % CV | 6.17% |
| % | 101.12% | % | 118.00% | % | 105.05% |
| Accuracy | | Accuracy | | Accuracy | |

Table 15 shows the within run precision of Lacosamide. The % CV for Lacosamide ranged from 5.78% to 3.26% across all three quality control levels.

LACOSAMIDE

| Low QC | 3 ug/mL | Medium QC | 6 ug/mL | High QC | 12 ug/mL |
|---|---|---|---|---|---|
| Run 1 | 2.82 | Run 1 | 6.44 | Run 1 | 13.42 |
| Run 2 | 3.18 | Run 2 | 6.79 | Run 2 | 13.36 |
| Run 3 | 3.01 | Run 3 | 6.33 | Run 3 | 13.24 |
| Run 4 | 3.08 | Run 4 | 6.5 | Run 4 | 14.12 |
| Run 5 | 3.01 | Run 5 | 6.51 | Run 5 | 14.07 |
| Run 6 | 3.06 | Run 6 | 6.43 | Run 6 | 14.23 |
| Run 7 | 2.78 | Run 7 | 6.88 | Run 7 | 14.13 |
| Run 8 | 3.10 | Run 8 | 6.54 | Run 8 | 12.04 |
| Run 9 | 3.06 | Run 9 | 6.51 | Run 9 | 13.84 |
| Run 10 | 3.06 | Run 10 | 6.98 | Run 10 | 12.28 |
| MEAN | 3.02 | MEAN | 6.59 | MEAN | 13.47 |
| STDEV | 0.12 | STDEV | 0.21 | STDEV | 0.78 |
| % CV | 4.11% | % CV | 3.26% | % CV | 5.78% |
| % | 100.53% | % Accuracy | 109.85% | % | 112.28% |
| Accuracy | | | | Accuracy | |

Table 16 shows the within run precision of Rufinamide. The % CV for Rufinamide ranged from 9.12% to 5.78% across all three quality control levels.

RUFINAMIDE

| Low QC | 7.5 ug/mL | Medium QC | 15 ug/mL | High QC | 30 ug/mL |
|---|---|---|---|---|---|
| Run 1 | 7.91 | Run 1 | 15.09 | Run 1 | 30.61 |
| Run 2 | 8.18 | Run 2 | 17.66 | Run 2 | 29.56 |
| Run 3 | 6.45 | Run 3 | 15.29 | Run 3 | 31.00 |
| Run 4 | 7.22 | Run 4 | 15.27 | Run 4 | 30.69 |
| Run 5 | 7.63 | Run 5 | 14.49 | Run 5 | 30.80 |
| Run 6 | 6.46 | Run 6 | 14.80 | Run 6 | 31.57 |
| Run 7 | 6.65 | Run 7 | 15.09 | Run 7 | 31.87 |
| Run 8 | 7.09 | Run 8 | 15.13 | Run 8 | 27.30 |
| Run 9 | 6.75 | Run 9 | 15.94 | Run 9 | 26.02 |
| Run 10 | 8.01 | Run 10 | 14.93 | Run 10 | 29.30 |
| MEAN | 7.24 | MEAN | 15.37 | MEAN | 29.87 |
| STDEV | 0.66 | STDEV | 0.89 | STDEV | 1.89 |
| % CV | 9.12% | % CV | 5.78% | % CV | 6.32% |
| % | 96.47% | % Accuracy | 102.46% | % | 99.57% |
| Accuracy | | | | Accuracy | |

Table 17 shows the within run precision of Felbamate. The % CV for Felbamate ranged from 8.63% to 5.89% across all three quality control levels.

FELBAMATE

| Low QC | 7.5 ug/mL | Medium QC | 15 ug/mL | High QC | 30 ug/mL |
|---|---|---|---|---|---|
| Run 1 | 7.2 | Run 1 | 15.09 | Run 1 | 28.06 |
| Run 2 | 7.82 | Run 2 | 16.29 | Run 2 | 29.69 |
| Run 3 | 6.65 | Run 3 | 14.05 | Run 3 | 29.82 |
| Run 4 | 7.48 | Run 4 | 15.41 | Run 4 | 32.43 |
| Run 5 | 6.78 | Run 5 | 15.02 | Run 5 | 36.25 |
| Run 6 | 6.53 | Run 6 | 15.35 | Run 6 | 35.02 |
| Run 7 | 6.96 | Run 7 | 14.65 | Run 7 | 31.45 |
| Run 8 | 7.38 | Run 8 | 13.20 | Run 8 | 28.42 |
| Run 9 | 7.39 | Run 9 | 13.78 | Run 9 | 29.92 |
| Run 10 | 7.51 | Run 10 | 14.70 | Run 10 | 30.81 |
| MEAN | 7.17 | MEAN | 14.75 | MEAN | 31.19 |
| STDEV | 0.42 | STDEV | 0.89 | STDEV | 2.69 |
| % CV | 5.89% | % CV | 6.06% | % CV | 8.63% |
| % | 95.60% | % Accuracy | 98.36% | % | 103.96% |
| Accuracy | | | | Accuracy | |

Table 18 shows the within run precision of 10,11 Carbamazepine Epoxide. The % CV for 10,11 Carbamazepine Epoxide ranged from 8.46% to 5.89% across all three quality control levels.

10, 11 CARBAMAZEPINE EPOXIDE

| | | Medium | | | |
|---|---|---|---|---|---|
| Low QC | 3.75 ug/mL | QC | 7.5 ug/mL | High QC | 15 ug/mL |
| Run 1 | 4.16 | Run 1 | 8.45 | Run 1 | 15.13 |
| Run 2 | 3.72 | Run 2 | 7.96 | Run 2 | 14.48 |
| Run 3 | 4.07 | Run 3 | 8.08 | Run 3 | 15.96 |
| Run 4 | 3.76 | Run 4 | 8.44 | Run 4 | 18.71 |
| Run 5 | 3.77 | Run 5 | 8.57 | Run 5 | 17.84 |
| Run 6 | 4.04 | Run 6 | 8.14 | Run 6 | 16.01 |
| Run 7 | 3.94 | Run 7 | 8.11 | Run 7 | 18.59 |
| Run 8 | 4.05 | Run 8 | 8.55 | Run 8 | 16.28 |
| Run 9 | 3.47 | Run 9 | 9.71 | Run 9 | 17.41 |
| Run 10 | 3.80 | Run 10 | 8.29 | Run 10 | 16.72 |
| MEAN | 3.88 | MEAN | 8.43 | MEAN | 16.71 |
| STDEV | 0.21 | STDEV | 0.50 | STDEV | 1.41 |
| % CV | 5.43% | % CV | 5.89% | % CV | 8.46% |
| % | 103.41% | % | 112.40% | % | 111.42% |
| Accuracy | | Accuracy | | Accuracy | |

Table 19 shows the within run precision of Phenytoin. The % CV for Phenytoin ranged from 8.40% to 7.26% across all three quality control levels.

PHENYTOIN

| Low QC | 3 ug/mL | Medium QC | 6 ug/mL | High QC | 12 ug/mL |
|---|---|---|---|---|---|
| Run 1 | 3.07 | Run 1 | 6.67 | Run 1 | 13.85 |
| Run 2 | 3.25 | Run 2 | 7.33 | Run 2 | 12.18 |
| Run 3 | 3.03 | Run 3 | 6.02 | Run 3 | 12.09 |
| Run 4 | 3.03 | Run 4 | 5.48 | Run 4 | 12.84 |
| Run 5 | 3.34 | Run 5 | 6.19 | Run 5 | 12.26 |
| Run 6 | 3.46 | Run 6 | 6.65 | Run 6 | 12.63 |
| Run 7 | 3.48 | Run 7 | 6.28 | Run 7 | 14.23 |
| Run 8 | 2.81 | Run 8 | 5.96 | Run 8 | 13.62 |
| Run 9 | 3.00 | Run 9 | 5.95 | Run 9 | 15.21 |
| Run 10 | 3.42 | Run 10 | 6.14 | Run 10 | 11.90 |
| MEAN | 3.19 | MEAN | 6.27 | MEAN | 13.08 |
| STDEV | 0.23 | STDEV | 0.51 | STDEV | 1.10 |
| % CV | 7.26% | % CV | 8.13% | % CV | 8.40% |
| % | 106.30% | % Accuracy | 104.45% | % | 109.01% |
| Accuracy | | | | Accuracy | |

Table 20 shows the within run precision of Carbamazepine. The % CV for Carbamazepine ranged from 9.45% to 4.93% across all three quality control levels.

CARBAMAZEPINE

| Low QC | 3 ug/mL | Medium QC | 6 ug/mL | High QC | 12 ug/mL |
|---|---|---|---|---|---|
| Run 1 | 3.04 | Run 1 | 7.75 | Run 1 | 12.06 |
| Run 2 | 3.40 | Run 2 | 7.55 | Run 2 | 13.01 |
| Run 3 | 3.50 | Run 3 | 6.85 | Run 3 | 14.25 |
| Run 4 | 3.38 | Run 4 | 7.62 | Run 4 | 13.24 |
| Run 5 | 3.38 | Run 5 | 6.46 | Run 5 | 12.99 |
| Run 6 | 3.15 | Run 6 | 6.82 | Run 6 | 15.38 |
| Run 7 | 3.57 | Run 7 | 7.22 | Run 7 | 16.19 |
| Run 8 | 3.25 | Run 8 | 6.71 | Run 8 | 13.27 |
| Run 9 | 3.43 | Run 9 | 7.48 | Run 9 | 13.28 |
| Run 10 | 3.49 | Run 10 | 6.56 | Run 10 | 12.52 |
| MEAN | 3.36 | MEAN | 7.10 | MEAN | 13.62 |
| STDEV | 0.17 | STDEV | 0.48 | STDEV | 1.29 |
| % CV | 4.93% | % CV | 6.72% | % CV | 9.45% |
| % | 111.97% | % Accuracy | 118.37% | % | 113.49% |
| Accuracy | | | | Accuracy | |

Table 21 shows the within run precision of Eslicarbamazepine. The % CV for Eslicarbamazepine ranged from 10.65% to 3.74% across all three quality control levels.

ESLICARBAMAZEPINE

| Low QC | 7.5 ug/mL |
|---|---|
| Run 1 | 7.36 |
| Run 2 | 7.72 |
| Run 3 | 7.17 |
| Run 4 | 6.99 |
| Run 5 | 7.83 |
| Run 6 | 7.32 |
| Run 7 | 7.18 |
| Run 8 | 7.35 |
| Run 9 | 7.07 |
| Run 10 | 7.56 |
| MEAN | 7.36 |
| STDEV | 0.28 |
| % CV | 3.74% |
| % Accuracy | 98.07% |
| Medium QC | 15 ug/mL |
| Run 1 | 16.12 |
| Run 2 | 15.59 |
| Run 3 | 15.63 |
| Run 4 | 15.19 |
| Run 5 | 15.28 |
| Run 6 | 16.54 |
| Run 7 | 16.10 |
| Run 8 | 15.80 |
| Run 9 | 14.06 |
| Run 10 | 13.08 |
| MEAN | 15.34 |
| STDEV | 1.04 |
| % CV | 6.79% |
| % Accuracy | 102.26% |
| High QC | 30 ug/mL |
| Run 1 | 31.41 |
| Run 2 | 27.55 |
| Run 3 | 34.19 |
| Run 4 | 36.35 |
| Run 5 | 30.75 |
| Run 6 | 27.83 |
| Run 7 | 33.70 |
| Run 8 | 38.61 |
| Run 9 | 31.75 |
| Run 10 | 32.54 |
| MEAN | 32.47 |
| STDEV | 3.46 |
| % CV | 10.65% |
| % Accuracy | 108.23% |

Table 22 shows the within run precision of Tiagabine. The % CV for Tiagabine ranged from 13.18% to 7.05% across all three quality control levels.

TIAGABINE

| Low QC | 0.03 ug/mL |
|---|---|
| Run 1 | 0.04 |
| Run 2 | 0.03 |
| Run 3 | 0.03 |
| Run 4 | 0.03 |
| Run 5 | 0.03 |
| Run 6 | 0.03 |
| Run 7 | 0.03 |
| Run 8 | 0.03 |
| Run 9 | 0.03 |
| Run 10 | 0.04 |
| MEAN | 0.03 |
| STDEV | 0.00 |
| % CV | 13.18% |
| % Accuracy | 0.43% |
| Medium QC | 0.06 ug/mL |
| Run 1 | 0.07 |
| Run 2 | 0.07 |
| Run 3 | 0.07 |
| Run 4 | 0.06 |
| Run 5 | 0.06 |
| Run 6 | 0.06 |
| Run 7 | 0.07 |
| Run 8 | 0.08 |
| Run 9 | 0.06 |
| Run 10 | 0.06 |
| MEAN | 0.07 |
| STDEV | 0.01 |
| % CV | 10.59% |
| % Accuracy | 0.44% |
| High QC | 0.12 ug/mL |
| Run 1 | 0.16 |
| Run 2 | 0.13 |
| Run 3 | 0.15 |
| Run 4 | 0.14 |
| Run 5 | 0.15 |
| Run 6 | 0.13 |
| Run 7 | 0.14 |
| Run 8 | 0.14 |
| Run 9 | 0.14 |
| Run 10 | 0.13 |
| MEAN | 0.14 |
| STDEV | 0.01 |
| % CV | 7.05% |
| % Accuracy | 0.47% |

Total run precision: Acceptability criteria: unacceptable if Total SD≧½TEa or Total SD must be less than a defined maximum SD or CV. The % CV should be less than allowable ≦TEa/2. The Tea for this assay is determined to be 30%.

The % CV for Ethosuximide ranged from 12.84% to 1.11% across all three quality control levels.

The % CV for Gabapentin ranged from 10.43% to 3.05% across all three quality control levels.

The % CV for Levetiracetam ranged from 8.48% to 2.28% across all three quality control levels.

The % CV for Pregabalin ranged from 10.21% to 2.43% across all three quality control levels.

The % CV for Zonisamide ranged from 12.44% to 1.44% across all three quality control levels.

The % CV for Lamotrigine ranged from 12.17% to 3.80% across all three quality control levels.

The % CV for Lacosamide ranged from 12.17% to 3.80% across all three quality control levels.

The % CV for Rufinamide ranged from 12.01% to 2.50% across all three quality control levels.

The % CV for Felbamate ranged from 7.92% to 2.03% across all three quality control levels.

The % CV for 10,11 Carbamazepine Epoxide ranged from 12.44% to 1.76% across all three quality control levels.

The % CV for Phenytoin ranged from 10.92% to 2.55% across all three quality control levels.

The % CV for Carbamazepine ranged from 12.64% to 2.05% across all three quality control levels.

The % CV for Eslicarbamazepine ranged from 13.49% to 3.60% across all three quality control levels.

The % CV for Tiagabine ranged from 16.11% to 0% across all three quality control levels.

Analytical sensitivity: Limit of Detection (LOD)—Calculation: LOD=mean of blank+4SD. The following are LODs: Ethosuximide-3.24 ng/ml; Levetiracetam-0.22 ng/ml; Pregabalin-0.29 ng/ml; Lamotrigine-0.17 ng/ml; Lacosamide-0.47 ng/ml.

Accuracy: Recovery of known standard—Acceptability criteria: the error due to lack of perfect recovery (amount recovered MINUS amount added) should be ≦2SD or 15% CV when TEa is 30%. Three whole blood samples were spiked at the following concentrations: 10, 30 and 60 ug/mL, each spike level was assayed in triplicate. There is no dilution analysis due to the way that whole blood is collected and dried on the Mitra microsampling device.

Table 23 shows accuracy of ethosuximide.

ETHOSUXIMIDE

| | | | | | |
|---|---|---|---|---|---|
| Repli- | Repli- | Repli- | | | Target |
| cate 1 | cate 2 | cate 3 | MEAN | STDEV | ug/mL |

Patient 1

| | | | | | | |
|---|---|---|---|---|---|---|
| Hemato- | 35.56 | 32.90 | 32.41 | 33.62 | 1.70 | 30.00 |
| crit 30% | | | | | | |
| Hemato- | 31.86 | 31.37 | 32.59 | 31.94 | 0.61 | 30.00 |
| crit 40% | | | | | | |
| Hemato- | 28.17 | 32.26 | 28.10 | 29.51 | 2.38 | 30.00 |
| crit 50% | | | | | | |
| Hemato- | 34.70 | 32.98 | 31.30 | 32.99 | 1.70 | 30.00 |
| crit 60% | | | | | | |
| | | | | Total | 32.02 | |
| | | | | Mean | | |
| | | | | Total | 1.60 | |
| | | | | RSD | | |
| | | | | % CV | 4.99% | |
| | | | | % | 106.72% | |
| | | | | Accuracy | | |

Patient 2

| | | | | | | |
|---|---|---|---|---|---|---|
| Hemato- | 51.76 | 63.8 | 57.13 | 57.56 | 6.03 | 60.00 |
| crit 30% | | | | | | |
| Hemato- | 55 | 53.66 | 52.46 | 53.71 | 1.27 | 60.00 |
| crit 40% | | | | | | |
| Hemato- | 58.01 | 57.74 | 48.06 | 54.60 | 5.67 | 60.00 |
| crit 50% | | | | | | |
| Hemato- | 49.23 | 54.06 | 53.21 | 52.17 | 2.58 | 60.00 |
| crit 60% | | | | | | |
| | | | | Total | 54.51 | |
| | | | | Mean | | |
| | | | | Total | 3.89 | |
| | | | | RSD | | |
| | | | | % CV | 7.13% | |
| | | | | % | 90.85% | |
| | | | | Accuracy | | |

Patient 3

| | | | | | | |
|---|---|---|---|---|---|---|
| Hemato- | 88.09 | 82.54 | 93.77 | 88.13 | 5.62 | 90.00 |
| crit 30% | | | | | | |
| Hemato- | 81.63 | 99.19 | 93.18 | 91.33 | 8.92 | 90.00 |
| crit 40% | | | | | | |
| Hemato- | 101.8 | 89.08 | 110.35 | 100.41 | 10.70 | 90.00 |
| crit 50% | | | | | | |
| Hemato- | 116.73 | 122.61 | 99.18 | 112.84 | 12.19 | 90.00 |
| crit 60% | | | | | | |
| | | | | Total | 98.18 | |
| | | | | Mean | | |
| | | | | Total | 9.36 | |
| | | | | RSD | | |
| | | | | % CV | 9.53% | |
| | | | | % | 109.09% | |
| | | | | Accuracy | | |

Table 24 shows accuracy of levetiracetam.

LEVETIRACETAM

| | | | | | |
|---|---|---|---|---|---|
| Repli- | Repli- | Repli- | | | Target |
| cate 1 | cate 2 | cate 3 | MEAN | STDEV | ug/mL |

Patient 1

| | | | | | | |
|---|---|---|---|---|---|---|
| Hemato- | 7.97 | 7.07 | 7.16 | 7.40 | 0.50 | 7.50 |
| crit 30% | | | | | | |
| Hemato- | 7.14 | 7.72 | 7.12 | 7.33 | 0.34 | 7.50 |
| crit 40% | | | | | | |
| Hemato- | 6.83 | 7.21 | 6.71 | 6.92 | 0.26 | 7.50 |
| crit 50% | | | | | | |
| Hemato- | 6.55 | 6.99 | 6.48 | 6.67 | 0.28 | 7.50 |
| crit 60% | | | | | | |
| | | | | Total | 7.08 | |
| | | | | Mean | | |
| | | | | Total | 0.34 | |
| | | | | RSD | | |
| | | | | % CV | 4.85% | |
| | | | | % | 94.39% | |
| | | | | Accuracy | | |

Patient 2

| | | | | | | |
|---|---|---|---|---|---|---|
| Hemato- | 10.3 | 11.8 | 11.93 | 11.34 | 0.91 | 12.00 |
| crit 30% | | | | | | |
| Hemato- | 11.09 | 10.93 | 10.38 | 10.80 | 0.37 | 12.00 |
| crit 40% | | | | | | |
| Hemato- | 9.99 | 11.45 | 10.82 | 10.75 | 0.73 | 12.00 |
| crit 50% | | | | | | |
| Hemato- | 8.82 | 11.9 | 11.29 | 10.67 | 1.63 | 12.00 |
| crit 60% | | | | | | |
| | | | | Total | 10.89 | |
| | | | | Mean | | |
| | | | | Total | 0.91 | |
| | | | | RSD | | |
| | | | | % CV | 8.36% | |
| | | | | % | 90.76% | |
| | | | | Accuracy | | |

Patient 3

| | | | | | | |
|---|---|---|---|---|---|---|
| Hemato- | 16.17 | 16.86 | 15.45 | 16.16 | 0.71 | 15.00 |
| crit 30% | | | | | | |
| Hemato- | 17.56 | 17.09 | 14.62 | 16.42 | 1.58 | 15.00 |
| crit 40% | | | | | | |
| Hemato- | 15.55 | 15.32 | 17.38 | 16.08 | 1.13 | 15.00 |
| crit 50% | | | | | | |
| Hemato- | 18.39 | 17.76 | 15.56 | 17.24 | 1.49 | 15.00 |
| crit 60% | | | | | | |
| | | | | Total | 16.48 | |
| | | | | Mean | | |
| | | | | Total | 1.22 | |
| | | | | RSD | | |
| | | | | % CV | 7.43% | |
| | | | | % | 109.84% | |
| | | | | Accuracy | | |

Table 25 shows accuracy of pregabalin.

PREGABALIN

| | | | | | |
|---|---|---|---|---|---|
| Repli- | Repli- | Repli- | | | Target |
| cate 1 | cate 2 | cate 3 | MEAN | STDEV | ug/mL |

Patient 1

| | | | | | | |
|---|---|---|---|---|---|---|
| Hemato- | 2.72 | 2.35 | 2.52 | 2.53 | 0.19 | 2.50 |
| crit 30% | | | | | | |
| Hemato- | 2.59 | 2.42 | 2.35 | 2.45 | 0.12 | 2.50 |
| crit 40% | | | | | | |
| Hemato- | 2.82 | 2.58 | 2.43 | 2.61 | 0.20 | 2.50 |
| crit 50% | | | | | | |
| Hemato- | 2.21 | 2.56 | 2.26 | 2.34 | 0.19 | 2.50 |
| crit 60% | | | | | | |
| | | | | Total | 2.48 | |
| | | | | Mean | | |
| | | | | Total | 0.17 | |
| | | | | RSD | | |
| | | | | % CV | 6.99% | |
| | | | | % | 99.37% | |
| | | | | Accuracy | | |

Patient 2

| | | | | | | |
|---|---|---|---|---|---|---|
| Hemato- | 3.75 | 4.64 | 4.59 | 4.33 | 0.50 | 4.50 |
| crit 30% | | | | | | |
| Hemato- | 4.05 | 4.33 | 4.13 | 4.17 | 0.14 | 4.50 |
| crit 40% | | | | | | |
| Hemato- | 3.47 | 4.42 | 3.54 | 3.81 | 0.53 | 4.50 |
| crit 50% | | | | | | |
| Hemato- | 3.21 | 4.13 | 3.87 | 3.74 | 0.47 | 4.50 |
| crit 60% | | | | | | |
| | | | | Total | 4.01 | |
| | | | | Mean | | |
| | | | | Total | 0.41 | |
| | | | | RSD | | |
| | | | | % CV | 10.27% | |
| | | | | % | 89.13% | |
| | | | | Accuracy | | |

Patient 3

| | | | | | | |
|---|---|---|---|---|---|---|
| Hemato- | 7.66 | 6.95 | 6.94 | 7.18 | 0.41 | 7.50 |
| crit 30% | | | | | | |
| Hemato- | 8.14 | 8.95 | 7.41 | 8.17 | 0.77 | 7.50 |
| crit 40% | | | | | | |
| Hemato- | 6.04 | 8.37 | 7.47 | 7.29 | 1.18 | 7.50 |
| crit 50% | | | | | | |
| Hemato- | 7.93 | 5.92 | 6.42 | 6.76 | 1.05 | 7.50 |
| crit 60% | | | | | | |
| | | | | Total | 7.35 | |
| | | | | Mean | | |
| | | | | Total | 0.85 | |
| | | | | RSD | | |
| | | | | % CV | 11.58% | |
| | | | | % | 98.00% | |
| | | | | Accuracy | | |

Table 26 shows accuracy of zonisamide.

ZONISAMIDE

| | | | | | |
|---|---|---|---|---|---|
| Repli- | Repli- | Repli- | | | Target |
| cate 1 | cate 2 | cate 3 | MEAN | STDEV | ug/mL |

Patient 1

| | | | | | | |
|---|---|---|---|---|---|---|
| Hemato- | 10.21 | 8.90 | 9.30 | 9.47 | 0.67 | 10.00 |
| crit 30% | | | | | | |
| Hemato- | 9.76 | 9.60 | 9.35 | 9.57 | 0.21 | 10.00 |
| crit 40% | | | | | | |
| Hemato- | 9.19 | 9.68 | 9.01 | 9.29 | 0.35 | 10.00 |
| crit 50% | | | | | | |
| Hemato- | 9.36 | 9.39 | 8.93 | 9.23 | 0.26 | 10.00 |
| crit 60% | | | | | | |
| | | | | Total | 9.39 | |
| | | | | Mean | | |
| | | | | Total | 0.37 | |
| | | | | RSD | | |
| | | | | % CV | 3.95% | |
| | | | | % | 93.90% | |
| | | | | Accuracy | | |

Patient 2

| | | | | | | |
|---|---|---|---|---|---|---|
| Hemato- | 14.46 | 16.25 | 15.9 | 15.54 | 0.95 | 15.00 |
| crit 30% | | | | | | |
| Hemato- | 14.99 | 13.79 | 15.02 | 14.60 | 0.70 | 15.00 |
| crit 40% | | | | | | |
| Hemato- | 12.83 | 14.96 | 13.94 | 13.91 | 1.07 | 15.00 |
| crit 50% | | | | | | |
| Hemato- | 14.09 | 16.29 | 15.77 | 15.38 | 1.15 | 15.00 |
| crit 60% | | | | | | |
| | | | | Total | 14.86 | |
| | | | | Mean | | |
| | | | | Total | 0.97 | |
| | | | | RSD | | |
| | | | | % CV | 6.50% | |
| | | | | % | 99.05% | |
| | | | | Accuracy | | |

Patient 3

| | | | | | | |
|---|---|---|---|---|---|---|
| Hemato- | 26.94 | 24.4 | 23.66 | 25.00 | 1.72 | 25.00 |
| crit 30% | | | | | | |
| Hemato- | 25.1 | 28.27 | 24.56 | 25.98 | 2.00 | 25.00 |
| crit 40% | | | | | | |
| Hemato- | 27.46 | 22.94 | 24.3 | 24.90 | 2.32 | 25.00 |
| crit 50% | | | | | | |
| Hemato- | 24.31 | 28.83 | 24.94 | 26.03 | 2.45 | 25.00 |
| crit 60% | | | | | | |
| | | | | Total | 25.48 | |
| | | | | Mean | | |
| | | | | Total | 2.12 | |
| | | | | RSD | | |
| | | | | % CV | 8.33% | |
| | | | | % | 101.90% | |
| | | | | Accuracy | | |

Table 27 shows accuracy of lamotrigine.

LAMOTRIGINE

| | | | | | |
|---|---|---|---|---|---|
| Repli- | Repli- | Repli- | | | Target |
| cate 1 | cate 2 | cate 3 | MEAN | STDEV | ug/mL |

Patient 1

| | | | | | | |
|---|---|---|---|---|---|---|
| Hemato- | 2.70 | 2.37 | 2.34 | 2.47 | 0.20 | 2.50 |
| crit 30% | | | | | | |
| Hemato- | 2.54 | 2.36 | 2.16 | 2.35 | 0.19 | 2.50 |
| crit 40% | | | | | | |
| Hemato- | 2.56 | 2.59 | 2.21 | 2.45 | 0.21 | 2.50 |
| crit 50% | | | | | | |
| Hemato- | 2.18 | 2.37 | 2.34 | 2.30 | 0.10 | 2.50 |
| crit 60% | | | | | | |
| | | | | Total | 2.39 | |
| | | | | Mean | | |
| | | | | Total | 0.18 | |
| | | | | RSD | | |
| | | | | % CV | 7.35% | |
| | | | | % | 95.73% | |
| | | | | Accuracy | | |

Patient 2

| | | | | | | |
|---|---|---|---|---|---|---|
| Hemato- | 3.68 | 4.13 | 3.91 | 3.91 | 0.23 | 3.75 |
| crit 30% | | | | | | |
| Hemato- | 3.65 | 3.62 | 3.62 | 3.63 | 0.02 | 3.75 |
| crit 40% | | | | | | |
| Hemato- | 3.36 | 3.98 | 3.46 | 3.60 | 0.33 | 3.75 |
| crit 50% | | | | | | |
| Hemato- | 3.86 | 4.25 | 3.97 | 4.03 | 0.20 | 3.75 |
| crit 60% | | | | | | |
| | | | | Total | 3.79 | |
| | | | | Mean | | |
| | | | | Total | 0.19 | |
| | | | | RSD | | |
| | | | | % CV | 5.12% | |
| | | | | % | 101.09% | |
| | | | | Accuracy | | |

Patient 3

| | | | | | | |
|---|---|---|---|---|---|---|
| Hemato- | 6.62 | 6.23 | 6.13 | 6.33 | 0.26 | 6.25 |
| crit 30% | | | | | | |
| Hemato- | 6.89 | 6.81 | 6.1 | 6.60 | 0.43 | 6.25 |
| crit 40% | | | | | | |
| Hemato- | 6.47 | 5.5 | 6.88 | 6.28 | 0.71 | 6.25 |
| crit 50% | | | | | | |
| Hemato- | 6.33 | 6.63 | 6.85 | 6.60 | 0.26 | 6.25 |
| crit 60% | | | | | | |
| | | | | Total | 6.45 | |
| | | | | Mean | | |
| | | | | Total | 0.42 | |
| | | | | RSD | | |
| | | | | % CV | 6.44% | |
| | | | | % | 103.25% | |
| | | | | Accuracy | | |

Table 28 shows accuracy of lacosamide.

LACOSAMIDE

| | | | | | |
|---|---|---|---|---|---|
| Repli- | Repli- | Repli- | | | Target |
| cate 1 | cate 2 | cate 3 | MEAN | STDEV | ug/mL |

Patient 1

| | | | | | | |
|---|---|---|---|---|---|---|
| Hemato- | 2.74 | 2.47 | 2.56 | 2.59 | 0.14 | 2.50 |
| crit 30% | | | | | | |
| Hemato- | 2.53 | 2.53 | 4.07 | 3.04 | 0.89 | 2.50 |
| crit 40% | | | | | | |
| Hemato- | 2.64 | 2.63 | 2.32 | 2.53 | 0.18 | 2.50 |
| crit 50% | | | | | | |
| Hemato- | 2.51 | 2.48 | 2.37 | 2.45 | 0.07 | 2.50 |
| crit 60% | | | | | | |
| | | | | Total | 2.65 | |
| | | | | Mean | | |
| | | | | Total | 0.32 | |
| | | | | RSD | | |
| | | | | % CV | 12.08% | |
| | | | | % | 106.17% | |
| | | | | Accuracy | | |

Patient 2

| | | | | | | |
|---|---|---|---|---|---|---|
| Hemato- | 3.86 | 4.21 | 4.02 | 4.03 | 0.18 | 4.00 |
| crit 30% | | | | | | |
| Hemato- | 3.92 | 3.9 | 2.4 | 3.41 | 0.87 | 4.00 |
| crit 40% | | | | | | |
| Hemato- | 3.63 | 4.34 | 3.73 | 3.90 | 0.38 | 4.00 |
| crit 50% | | | | | | |
| Hemato- | 3.85 | 4.03 | 3.97 | 3.95 | 0.09 | 4.00 |
| crit 60% | | | | | | |
| | | | | Total | 3.82 | |
| | | | | Mean | | |
| | | | | Total | 0.38 | |
| | | | | RSD | | |
| | | | | % CV | 9.96% | |
| | | | | % | 95.54% | |
| | | | | Accuracy | | |

Patient 3

| | | | | | | |
|---|---|---|---|---|---|---|
| Hemato- | 6.77 | 6.26 | 6.82 | 6.62 | 0.31 | 6.25 |
| crit 30% | | | | | | |
| Hemato- | 7.32 | 6.77 | 6.37 | 6.82 | 0.48 | 6.25 |
| crit 40% | | | | | | |
| Hemato- | 6.5 | 5.73 | 6.68 | 6.30 | 0.50 | 6.25 |
| crit 50% | | | | | | |
| Hemato- | 6.16 | 6.72 | 6.04 | 6.31 | 0.36 | 6.25 |
| crit 60% | | | | | | |
| | | | | Total | 6.51 | |
| | | | | Mean | | |
| | | | | Total | 0.41 | |
| | | | | RSD | | |
| | | | | % CV | 6.35% | |
| | | | | % | 104.19% | |
| | | | | Accuracy | | |

Table 29 shows accuracy of rufinamide.

RUFINAMIDE

| | | | | | |
|---|---|---|---|---|---|
| Repli- | Repli- | Repli- | | | Target |
| cate 1 | cate 2 | cate 3 | MEAN | STDEV | ug/mL |

Patient 1

| | | | | | | |
|---|---|---|---|---|---|---|
| Hemato- | 5.12 | 4.59 | 4.61 | 4.77 | 0.21 | 5.00 |
| crit 30% | | | | | | |
| Hemato- | 4.93 | 4.84 | 4.53 | 4.75 | 0.75 | 5.00 |
| crit 40% | | | | | | |
| Hemato- | 3.89 | 5.06 | 5.29 | 4.94 | 0.26 | 5.00 |
| crit 50% | | | | | | |
| Hemato- | 4.84 | 5.23 | 4.74 | 4.94 | 0.26 | 5.00 |
| crit 60% | | | | | | |
| | | | | Total | 4.85 | |
| | | | | Mean | | |
| | | | | Total | 0.37 | |
| | | | | RSD | | |
| | | | | % CV | 7.63% | |
| | | | | % | 96.93% | |
| | | | | Accuracy | | |

Patient 2

| | | | | | | |
|---|---|---|---|---|---|---|
| Hemato- | 8.52 | 7.26 | 6.51 | 8.35 | 0.26 | 7.50 |
| crit 30% | | | | | | |
| Hemato- | 8.61 | 8.36 | 8.09 | 7.76 | 1.15 | 7.50 |
| crit 40% | | | | | | |
| Hemato- | 6.52 | 8.80 | 7.97 | 9.08 | 0.65 | 7.50 |
| crit 50% | | | | | | |
| Hemato- | 8.34 | 9.35 | 9.56 | 9.08 | 0.65 | 7.50 |
| crit 60% | | | | | | |
| | | | | Total | 8.57 | |
| | | | | Mean | | |
| | | | | Total | 0.68 | |
| | | | | RSD | | |
| | | | | % CV | 7.93% | |
| | | | | % | 114.28% | |
| | | | | Accuracy | | |

Patient 1

| | | | | | | |
|---|---|---|---|---|---|---|
| Hemato- | 11.55 | 10.77 | 11.31 | 11.20 | 0.58 | 10.00 |
| crit 30% | | | | | | |
| Hemato- | 11.36 | 10.56 | 11.69 | 9.88 | 0.53 | 10.00 |
| crit 40% | | | | | | |
| Hemato- | 9.67 | 9.49 | 10.48 | 10.59 | 0.89 | 10.00 |
| crit 50% | | | | | | |
| Hemato- | 10.32 | 11.58 | 9.87 | 10.59 | 0.89 | 10.00 |
| crit 60% | | | | | | |
| | | | | Total | 10.57 | |
| | | | | Mean | | |
| | | | | Total | 0.72 | |
| | | | | RSD | | |
| | | | | % CV | 6.82% | |
| | | | | % | 105.66% | |
| | | | | Accuracy | | |

Table 30 shows accuracy of felbamate.

FELBAMATE

| | | | | | |
|---|---|---|---|---|---|
| Repli- | Repli- | Repli- | | | Target |
| cate 1 | cate 2 | cate 3 | MEAN | STDEV | ug/mL |

Patient 1

| | | | | | | |
|---|---|---|---|---|---|---|
| Hemato- | 6.30 | 6.01 | 5.84 | 6.05 | 0.23 | 5.00 |
| crit 30% | | | | | | |
| Hemato- | 6.00 | 6.14 | 5.28 | 5.81 | 0.46 | 5.00 |
| crit 40% | | | | | | |
| Hemato- | 4.58 | 5.75 | 4.46 | 4.93 | 0.71 | 5.00 |
| crit 50% | | | | | | |
| Hemato- | 5.55 | 4.35 | 5.09 | 5.00 | 0.61 | 5.00 |
| crit 60% | | | | | | |
| | | | | Total | 5.45 | |
| | | | | Mean | | |
| | | | | Total | 0.50 | |
| | | | | RSD | | |
| | | | | % CV | 9.24% | |
| | | | | % | 108.92% | |
| | | | | Accuracy | | |

Patient 2

| | | | | | | |
|---|---|---|---|---|---|---|
| Hemato- | 8.81 | 8.44 | 6.65 | 7.97 | 1.16 | 7.50 |
| crit 30% | | | | | | |
| Hemato- | 8.14 | 7.84 | 8.22 | 8.07 | 0.20 | 7.50 |
| crit 40% | | | | | | |
| Hemato- | 8.38 | 8.75 | 6.78 | 7.97 | 1.05 | 7.50 |
| crit 50% | | | | | | |
| Hemato- | 7.93 | 8.99 | 7.94 | 8.29 | 0.61 | 7.50 |
| crit 60% | | | | | | |
| | | | | Total | 8.07 | |
| | | | | Mean | | |
| | | | | Total | 0.75 | |
| | | | | RSD | | |
| | | | | % CV | 9.33% | |
| | | | | % | 107.63% | |
| | | | | Accuracy | | |

Patient 3

| | | | | | | |
|---|---|---|---|---|---|---|
| Hemato- | 10.57 | 10.05 | 11.8 | 10.81 | 0.90 | 10.00 |
| crit 30% | | | | | | |
| Hemato- | 11.7 | 10.99 | 10.56 | 11.08 | 0.58 | 10.00 |
| crit 40% | | | | | | |
| Hemato- | 11.71 | 11.42 | 12.15 | 11.76 | 0.37 | 10.00 |
| crit 50% | | | | | | |
| Hemato- | 11.51 | 11.63 | 11.02 | 11.39 | 0.32 | 10.00 |
| crit 60% | | | | | | |
| | | | | Total | 11.26 | |
| | | | | Mean | | |
| | | | | Total | 0.54 | |
| | | | | RSD | | |
| | | | | % CV | 4.81% | |
| | | | | % | 112.59% | |
| | | | | Accuracy | | |

Table 31 shows accuracy of carbamzepine.

1011 CARBAMZEPINE EPOXIDE

| | | | | | |
|---|---|---|---|---|---|
| Repli- | Repli- | Repli- | | | Target |
| cate 1 | cate 2 | cate 3 | MEAN | STDEV | ug/mL |

Patient 1

| | | | | | | |
|---|---|---|---|---|---|---|
| Hemato- | 2.73 | 2.07 | 2.49 | 2.43 | 0.33 | 2.50 |
| crit 30% | | | | | | |
| Hemato- | 2.57 | 2.06 | 2.27 | 2.30 | 0.25 | 2.50 |
| crit 40% | | | | | | |
| Hemato- | 2.59 | 2.29 | 2.30 | 2.39 | 0.17 | 2.50 |
| crit 50% | | | | | | |
| Hemato- | 2.21 | 2.07 | 2.56 | 2.28 | 0.25 | 2.50 |
| crit 60% | | | | | | |
| | | | | Total | 2.35 | |
| | | | | Mean | | |
| | | | | Total | 0.25 | |
| | | | | RSD | | |
| | | | | % CV | 10.71% | |
| | | | | % | 93.97% | |
| | | | | Accuracy | | |

Patient 2

| | | | | | | |
|---|---|---|---|---|---|---|
| Hemato- | 3.76 | 4.21 | 3.99 | 3.99 | 0.23 | 3.75 |
| crit 30% | | | | | | |
| Hemato- | 3.75 | 3.72 | 3.72 | 3.73 | 0.02 | 3.75 |
| crit 40% | | | | | | |
| Hemato- | 3.76 | 4.15 | 3.87 | 3.93 | 0.20 | 3.75 |
| crit 50% | | | | | | |
| Hemato- | 3.41 | 4.03 | 3.51 | 3.65 | 0.33 | 3.75 |
| crit 60% | | | | | | |
| | | | | Total | 3.82 | |
| | | | | Mean | | |
| | | | | Total | 0.19 | |
| | | | | RSD | | |
| | | | | % CV | 5.08% | |
| | | | | % | 101.96% | |
| | | | | Accuracy | | |

Patient 3

| | | | | | | |
|---|---|---|---|---|---|---|
| Hemato- | 6.76 | 7.1 | 6.88 | 6.91 | 0.17 | 6.25 |
| crit 30% | | | | | | |
| Hemato- | 6.75 | 6.84 | 6.84 | 6.81 | 0.05 | 6.25 |
| crit 40% | | | | | | |
| Hemato- | 6.76 | 6.04 | 5.76 | 6.19 | 0.52 | 6.25 |
| crit 50% | | | | | | |
| Hemato- | 6.41 | 6.26 | 5.74 | 6.14 | 0.35 | 6.25 |
| crit 60% | | | | | | |
| | | | | Total | 6.51 | |
| | | | | Mean | | |
| | | | | Total | 0.27 | |
| | | | | RSD | | |
| | | | | % CV | 4.19% | |
| | | | | % | 104.19% | |
| | | | | Accuracy | | |

Table 32 shows accuracy of phenytoin.

PHENYTOIN

| | | | | | |
|---|---|---|---|---|---|
| Repli- | Repli- | Repli- | | | Target |
| cate 1 | cate 2 | cate 3 | MEAN | STDEV | ug/mL |

Patient 1

| | | | | | | |
|---|---|---|---|---|---|---|
| Hemato- | 8.14 | 8.11 | 7.65 | 7.97 | 0.27 | 7.50 |
| crit 30% | | | | | | |
| Hemato- | 7.06 | 7.19 | 7.12 | 7.12 | 0.07 | 7.50 |
| crit 40% | | | | | | |
| Hemato- | 6.51 | 7.95 | 6.45 | 6.97 | 0.85 | 7.50 |
| crit 50% | | | | | | |
| Hemato- | 6.90 | 6.98 | 6.31 | 6.73 | 0.37 | 7.50 |
| crit 60% | | | | | | |
| | | | | Total | 7.20 | |
| | | | | Mean | | |
| | | | | Total | 0.39 | |
| | | | | RSD | | |
| | | | | % CV | 5.40% | |
| | | | | % | 95.97% | |
| | | | | Accuracy | | |

Patient 2

| | | | | | | |
|---|---|---|---|---|---|---|
| Hemato- | 12.42 | 14.45 | 12.7 | 13.19 | 1.10 | 13.00 |
| crit 30% | | | | | | |
| Hemato- | 12.21 | 12.59 | 11.13 | 11.98 | 0.76 | 13.00 |
| crit 40% | | | | | | |
| Hemato- | 10.78 | 12.68 | 9.86 | 11.11 | 1.44 | 13.00 |
| crit 50% | | | | | | |
| Hemato- | 11.63 | 12.48 | 14.62 | 12.91 | 1.54 | 13.00 |
| crit 60% | | | | | | |
| | | | | Total | 12.30 | |
| | | | | Mean | | |
| | | | | Total | 1.21 | |
| | | | | RSD | | |
| | | | | % CV | 9.83% | |
| | | | | % | 94.58% | |
| | | | | Accuracy | | |

Patient 3

| | | | | | | |
|---|---|---|---|---|---|---|
| Hemato- | 18.79 | 18.45 | 16.7 | 17.98 | 1.12 | 18.00 |
| crit 30% | | | | | | |
| Hemato- | 18.37 | 16.43 | 18.38 | 17.73 | 1.12 | 18.00 |
| crit 40% | | | | | | |
| Hemato- | 15.51 | 19.03 | 19.54 | 18.03 | 2.19 | 18.00 |
| crit 50% | | | | | | |
| Hemato- | 17.21 | 15.81 | 19.74 | 17.59 | 1.99 | 18.00 |
| crit 60% | | | | | | |
| | | | | Total | 17.83 | |
| | | | | Mean | | |
| | | | | Total | 1.61 | |
| | | | | RSD | | |
| | | | | % CV | 9.02% | |
| | | | | % | 99.06% | |
| | | | | Accuracy | | |

Table 33 shows accuracy of carbamazepine.

CARBAMAZEPINE

| | | | | | |
|---|---|---|---|---|---|
| Repli- | Repli- | Repli- | | | Target |
| cate 1 | cate 2 | cate 3 | MEAN | STDEV | ug/mL |

Patient 1

| | | | | | | |
|---|---|---|---|---|---|---|
| Hemato- | 3.03 | 2.95 | 3.18 | 3.05 | 0.12 | 2.75 |
| crit 30% | | | | | | |
| Hemato- | 2.87 | 2.90 | 2.68 | 2.82 | 0.12 | 2.75 |
| crit 40% | | | | | | |
| Hemato- | 3.04 | 3.06 | 2.32 | 2.81 | 0.42 | 2.75 |
| crit 50% | | | | | | |
| Hemato- | 2.40 | 2.89 | 2.73 | 2.67 | 0.25 | 2.75 |
| crit 60% | | | | | | |
| | | | | Total | 2.84 | |
| | | | | Mean | | |
| | | | | Total | 0.23 | |
| | | | | RSD | | |
| | | | | % CV | 8.00% | |
| | | | | % | 103.18% | |
| | | | | Accuracy | | |

Patient 2

| | | | | | | |
|---|---|---|---|---|---|---|
| Hemato- | 4.13 | 4.99 | 4.59 | 4.57 | 0.43 | 4.50 |
| crit 30% | | | | | | |
| Hemato- | 4.23 | 4.31 | 4.02 | 4.19 | 0.15 | 4.50 |
| crit 40% | | | | | | |
| Hemato- | 4.10 | 4.34 | 3.99 | 4.14 | 0.18 | 4.50 |
| crit 50% | | | | | | |
| Hemato- | 4.74 | 4.91 | 4.50 | 4.72 | 0.21 | 4.50 |
| crit 60% | | | | | | |
| | | | | Total | 4.40 | |
| | | | | Mean | | |
| | | | | Total | 0.24 | |
| | | | | RSD | | |
| | | | | % CV | 5.48% | |
| | | | | % | 97.87% | |
| | | | | Accuracy | | |

Patient 3

| | | | | | | |
|---|---|---|---|---|---|---|
| Hemato- | 6.85 | 7.1 | 6.81 | 6.92 | 0.16 | 6.85 |
| crit 30% | | | | | | |
| Hemato- | 7.37 | 7.03 | 6.65 | 7.02 | 0.36 | 6.85 |
| crit 40% | | | | | | |
| Hemato- | 7.06 | 6.67 | 8.26 | 7.33 | 0.83 | 6.85 |
| crit 50% | | | | | | |
| Hemato- | 7.34 | 7.56 | 7.34 | 7.41 | 0.13 | 6.85 |
| crit 60% | | | | | | |
| | | | | Total | 7.17 | |
| | | | | Mean | | |
| | | | | Total | 0.37 | |
| | | | | RSD | | |
| | | | | % CV | 5.14% | |
| | | | | % | 104.67% | |
| | | | | Accuracy | | |

Table 34 shows accuracy of eslicarbamazepine.

ESLICARBAMAZEPINE

| | | | | | |
|---|---|---|---|---|---|
| Repli- | Repli- | Repli- | | | Target |
| cate 1 | cate 2 | cate 3 | MEAN | STDEV | ug/mL |

Patient 1

| | | | | | | |
|---|---|---|---|---|---|---|
| Hemato- | 8.02 | 5.70 | 6.72 | 6.81 | 1.16 | 6.50 |
| crit 30% | | | | | | |
| Hemato- | 6.81 | 6.90 | 6.44 | 6.72 | 0.24 | 6.50 |
| crit 40% | | | | | | |
| Hemato- | 6.35 | 6.60 | 5.90 | 6.28 | 0.35 | 6.50 |
| crit 50% | | | | | | |
| Hemato- | 6.89 | 6.87 | 6.78 | 6.85 | 0.06 | 6.50 |
| crit 60% | | | | | | |
| | | | | Total | 6.67 | |
| | | | | Mean | | |
| | | | | Total | 0.45 | |
| | | | | RSD | | |
| | | | | % CV | 6.83% | |
| | | | | % | 102.54% | |
| | | | | Accuracy | | |

Patient 2

| | | | | | | |
|---|---|---|---|---|---|---|
| Hemato- | 10.99 | 12.27 | 10.81 | 11.36 | 0.80 | 12.00 |
| crit 30% | | | | | | |
| Hemato- | 10.79 | 10.91 | 10.75 | 10.82 | 0.08 | 12.00 |
| crit 40% | | | | | | |
| Hemato- | 9.31 | 12.51 | 9.62 | 10.48 | 1.76 | 12.00 |
| crit 50% | | | | | | |
| Hemato- | 10.14 | 10.8 | 10.69 | 10.54 | 0.35 | 12.00 |
| crit 60% | | | | | | |
| | | | | Total | 10.80 | |
| | | | | Mean | | |
| | | | | Total | 0.75 | |
| | | | | RSD | | |
| | | | | % CV | 6.94% | |
| | | | | % | 89.99% | |
| | | | | Accuracy | | |

Patient 3

| | | | | | | |
|---|---|---|---|---|---|---|
| Hemato- | 18.21 | 17.23 | 16.58 | 17.34 | 0.82 | 18.00 |
| crit 30% | | | | | | |
| Hemato- | 17.59 | 19.69 | 16.23 | 17.84 | 1.74 | 18.00 |
| crit 40% | | | | | | |
| Hemato- | 17.81 | 16.75 | 19.4 | 17.99 | 1.33 | 18.00 |
| crit 50% | | | | | | |
| Hemato- | 16.58 | 17.8 | 17.11 | 17.16 | 0.61 | 18.00 |
| crit 60% | | | | | | |
| | | | | Total | 17.58 | |
| | | | | Mean | | |
| | | | | Total | 1.13 | |
| | | | | RSD | | |
| | | | | % CV | 6.41% | |
| | | | | % | 97.68% | |
| | | | | Accuracy | | |

Table 35 shows accuracy of tiagabine.

TIAGABINE

| | | | | | |
|---|---|---|---|---|---|
| Repli- | Repli- | Repli- | | | Target |
| cate 1 | cate 2 | cate 3 | MEAN | STDEV | ug/mL |

Patient 1

| | | | | | | |
|---|---|---|---|---|---|---|
| Hemato- | 0.11 | 0.10 | 0.10 | 0.10 | 0.01 | 0.10 |
| crit 30% | | | | | | |
| Hemato- | 0.10 | 0.10 | 0.10 | 0.10 | 0.00 | 0.10 |
| crit 40% | | | | | | |
| Hemato- | 0.10 | 0.11 | 0.09 | 0.10 | 0.01 | 0.10 |
| crit 50% | | | | | | |
| Hemato- | 0.08 | 0.10 | 0.09 | 0.09 | 0.01 | 0.10 |
| crit 60% | | | | | | |
| | | | | Total | 0.10 | |
| | | | | Mean | | |
| | | | | Total | 0.01 | |
| | | | | RSD | | |
| | | | | % CV | 6.55% | |
| | | | | % | 98.33% | |
| | | | | Accuracy | | |

Patient 2

| | | | | | | |
|---|---|---|---|---|---|---|
| Hemato- | 0.17 | 0.16 | 0.19 | 0.17 | 0.02 | 0.15 |
| crit 30% | | | | | | |
| Hemato- | 0.16 | 0.18 | 0.16 | 0.17 | 0.01 | 0.15 |
| crit 40% | | | | | | |
| Hemato- | 0.15 | 0.21 | 0.14 | 0.17 | 0.04 | 0.15 |
| crit 50% | | | | | | |
| Hemato- | 0.15 | 0.18 | 0.15 | 0.16 | 0.02 | 0.15 |
| crit 60% | | | | | | |
| | | | | Total | 0.17 | |
| | | | | Mean | | |
| | | | | Total | 0.02 | |
| | | | | RSD | | |
| | | | | % CV | 12.30% | |
| | | | | % | 98.04% | |
| | | | | Accuracy | | |

Patient 3

| | | | | | | |
|---|---|---|---|---|---|---|
| Hemato- | 0.36 | 0.35 | 0.31 | 0.34 | 0.03 | 0.20 |
| crit 30% | | | | | | |
| Hemato- | 0.32 | 0.32 | 0.29 | 0.31 | 0.02 | 0.20 |
| crit 40% | | | | | | |
| Hemato- | 0.28 | 0.27 | 0.32 | 0.29 | 0.03 | 0.20 |
| crit 50% | | | | | | |
| Hemato- | 0.29 | 0.31 | 0.29 | 0.30 | 0.01 | 0.20 |
| crit 60% | | | | | | |
| | | | | Total | 0.31 | |
| | | | | Mean | | |
| | | | | Total | 0.02 | |
| | | | | RSD | | |
| | | | | % CV | 6.61% | |
| | | | | % | 0.34% | |
| | | | | Accuracy | | |

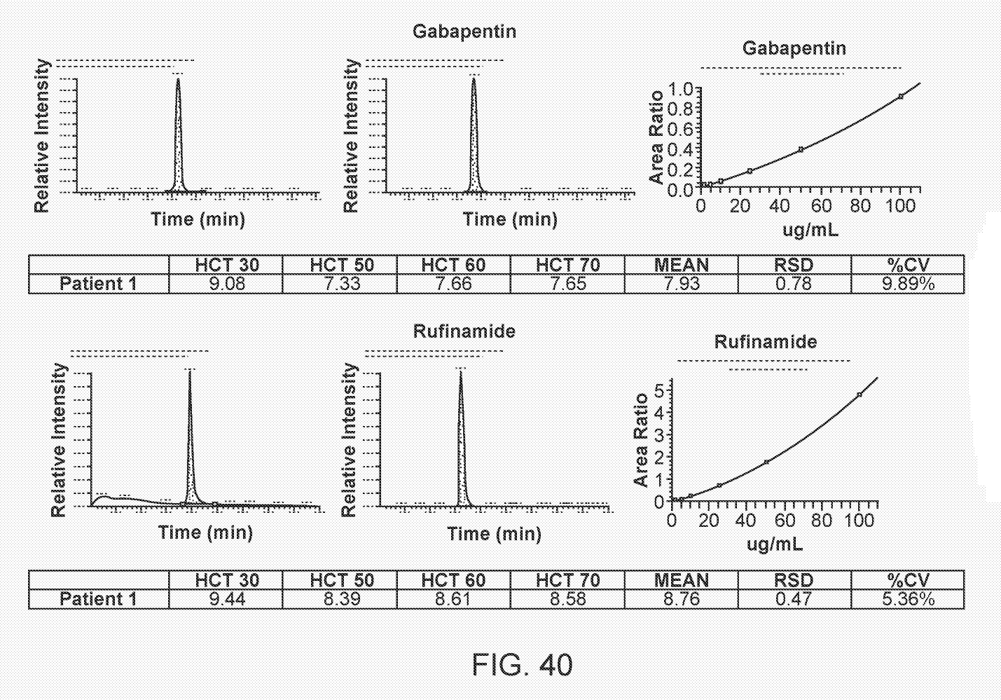

FIG. 40 shows the results of hematocrit study of gabapentin and rufinamide.

### Example 8

### 250H Hydroxy Vitamin D

In this assay, patient whole blood samples were analyzed.

Vitamin D in human blood was extracted from a 20 uL Mitra microsampling device by adding 10 uL of internal standard and 500 uL of the extraction solvent (1M ammonium hydroxide solution in 50:50 ethyl acetate and methanol) into a clean 96-well plate. The Mitra tips were dropped into the wells with the IS/extraction solvent mixture. The plate was mixed in a heated plate mixer/vortexer at 800 rpm for one hour at 45° C. (Eppendorf mixmate). The extraction solvent in the mixed sample plate was dried down under heated nitrogen @ 60° C. for ˜15 minutes to concentrate the sample. When dry down was complete, 100 uL of 0.1 ng/mL of the derivatization reagent (PTAD) in acetonitrile was added to the sample wells and incubated at room temperature for one hour. 100 uL of HPLC grade water was added to the wells to quench the reaction. The samples were then transferred to a 96-well filter plate (Captiva ND) with a clean 96-well collection plate secured underneath it. Positive pressure was applied to the filter plate to allow the filtrate to go through. 25 uL of the sample ws injected into the LC-MSMS system.

Separation was achieved by using a reverse-phase C18 column and mobile phase which consists of 0.1% aqueous formic acid (mobile phase A) and 50:50 methanol and acetonitrile (mobile phase B). The Aria LX-system equipped with Agilent SL pumps were coupled to a TSQ Quantum Ultra triple quadrupole mass spectrometer as a detector, with a heated electrospray (HESI) source. 25-hydroxyvitamin D2 and D3 were detected and quantitated on positive ionization mode MRM/SRM scan. The following parameters were used: Ionization Voltage 5000 V; Vaporizer Temperature 450° C.; Sheath Gas 20 Arb; Aux 20 Arb; Collision Pressure 1.0 mTorr; Collision Energy 16-18 V.

The following mass transitions are monitored:

| | Parent | Fragment | Retention Time |
|---|---|---|---|
| Analyte | Mass | Mass | (mins) |
| 25OHD2 | 570.3 | 298.1 | 1.11 |
| 25OHD2-d₃ | 573.3 | 301.1 | 1.11 |
| 25OHD3 | 558.3 | 298.2 | 1.07 |
| 25OHD3-d₃ | 561.3 | 301.2 | 1.07 |

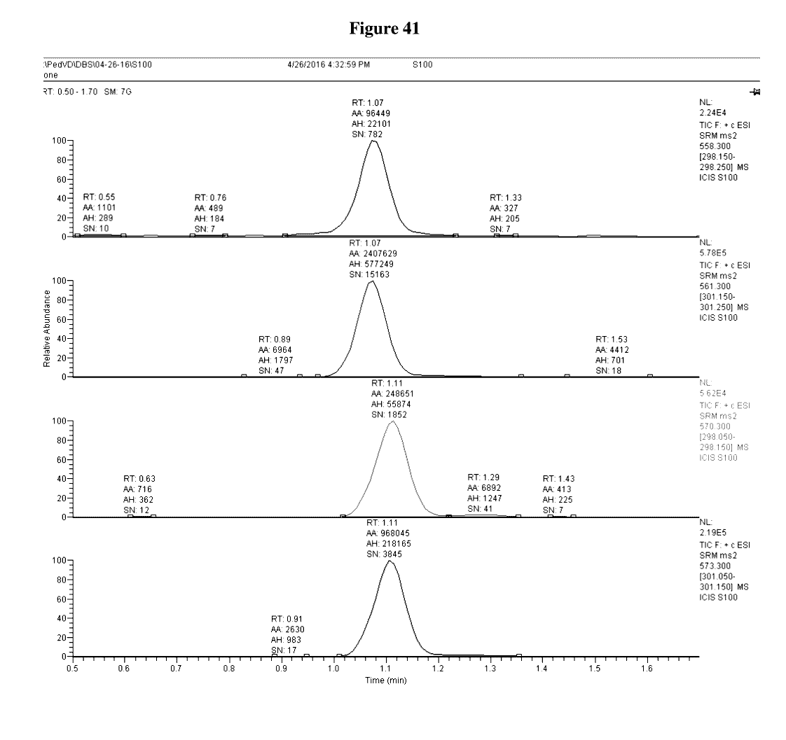

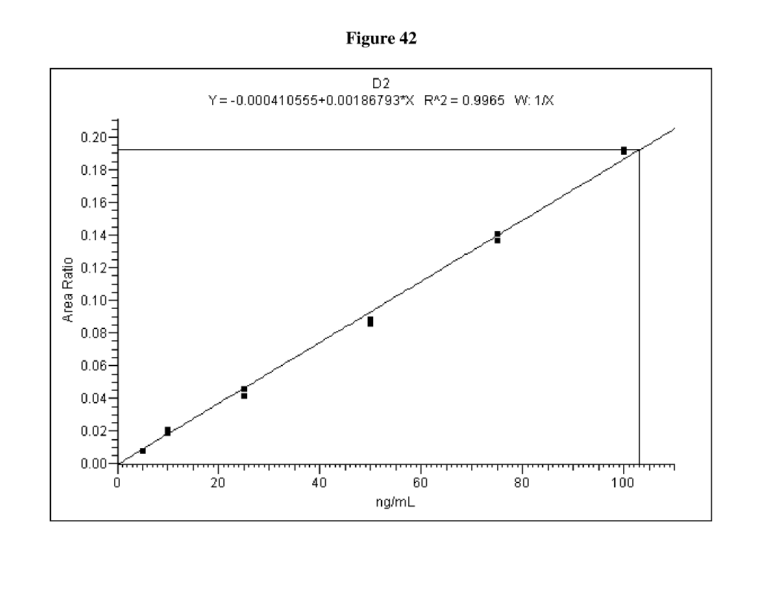

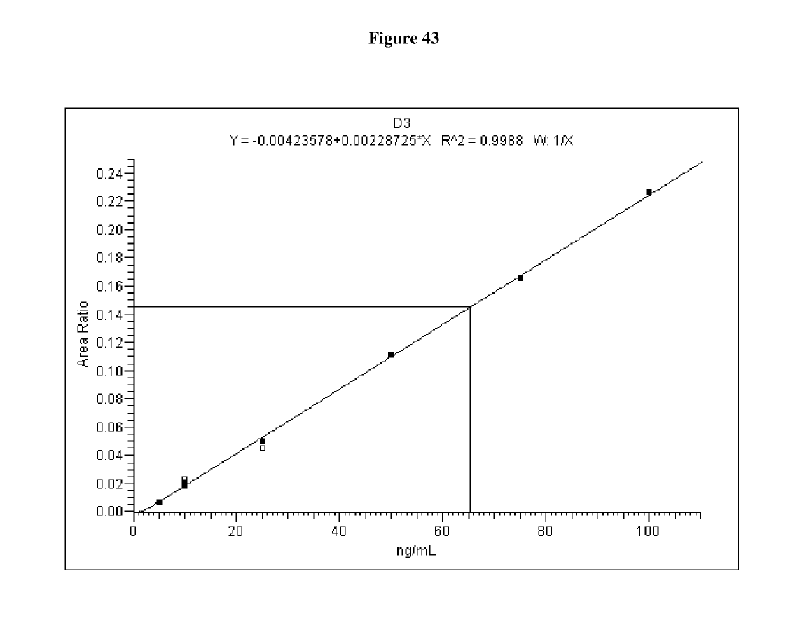

FIG. 41 shows the chromatogram of the 25-hydroxyvitamin D analysis. FIG. 42 shows the calibration curve of 25-hydroxyvitamin D2 analysis. FIG. 43 shows the calibration curve of 25-hydroxyvitamin D3 analysis.

The linear range of analysis was 5-100 ng/ml. The limit of quantitation (LOQ) was 4 ng/ml.

The contents of the articles, patents, and patent applications, and all other documents and electronically available information mentioned or cited herein, are hereby incorporated by reference in their entirety to the same extent as if each individual publication was specifically and individually indicated to be incorporated by reference. Applicants reserve the right to physically incorporate into this application any and all materials and information from any such articles, patents, patent applications, or other physical and electronic documents.

The methods illustratively described herein may suitably be practiced in the absence of any element or elements, limitation or limitations, not specifically disclosed herein. Thus, for example, the terms “comprising”, “including,” containing”, etc. shall be read expansively and without limitation. Additionally, the terms and expressions employed herein have been used as terms of description and not of limitation, and there is no intention in the use of such terms and expressions of excluding any equivalents of the features shown and described or portions thereof. It is recognized that various modifications are possible within the scope of the invention claimed. Thus, it should be understood that although the present invention has been specifically disclosed by preferred embodiments and optional features, modification and variation of the invention embodied therein herein disclosed may be resorted to by those skilled in the art, and that such modifications and variations are considered to be within the scope of this invention.

The invention has been described broadly and generically herein. Each of the narrower species and subgeneric groupings falling within the generic disclosure also form part of the methods. This includes the generic description of the methods with a proviso or negative limitation removing any subject matter from the genus, regardless of whether or not the excised material is specifically recited herein.

Other embodiments are within the following claims. In addition, where features or aspects of the methods are described in terms of Markush groups, those skilled in the art will recognize that the invention is also thereby described in terms of any individual member or subgroup of members of the Markush group.

That which is claimed is:

1. A method for determining the amount of an analyte in a sample by mass spectrometry, the method comprising:
(a) extracting an analyte from a sample collected by a microsampling device;
(b) ionizing the analyte to generate one or more ions detectable by mass spectrometry; and
(c) determining the amount of the one or more ions by mass spectrometry;
wherein the amount of the one or more ions determined is used to determine the amount of analyte in the sample.

2. The method of claim 1, wherein the amount of analyte in the sample is related to the amount of analyte in the patient.

3. The method of claim 1, wherein said sample comprises a whole blood, urine, saliva, plasma or serum sample.

4. The method of claim 1, wherein the extracting step comprises adding an extraction buffer to the sample collected by a microsampling device.

5. The method of claim 4, wherein the extracting step comprises drying down under nitrogen gas.

6. The method of claim 1, wherein the extracting step comprises reconstituting the sample into solution.

7. The method of claim 1, wherein the microsampling device comprises an apparatus that enables automation of extraction and mass spectrometric analysis of multiple samples at the same time.

8. The method of claim 1, wherein the extraction and mass spectrometry steps are performed in an on-line fashion to allow for automated sample analysis.

9. The method of claim 1, the sample collected by the microsampling device has a volume of less than or equal to 100 pt.

10. The method of claim 1, wherein the sample collected by the microsampling device has a volume of less than or equal to 50 pt.

11. The method of claim 1, wherein the sample collected by the microsampling device has a volume of about 10 μL, about 15 μL, or about 20 pt.

12. The method of claim 1, wherein the sample is hydrolyzed prior to quantitation by mass spectrometry.

13. The method of claim 1, further comprising purifying the sample prior to mass spectrometry.

14. The method of claim 1, wherein said purifying comprises subjecting the sample to liquid chromatography.

15. The method of claim 14, wherein liquid chromatography comprises high performance liquid chromatography (HPLC) or high turbulence liquid chromatograph (HTLC).

16. The method of claim 1, wherein the sample is capillary blood.

17. The method of claim 1, wherein the mass spectrometry is tandem mass spectrometry.

18. The method of claim 1, wherein ionization is atmospheric pressure chemical ionization (APCI).

19. The method of claim 1, wherein ionization is in positive ion mode.

20. The method of claim 1, wherein an internal standard for said analyte is added to the sample.

21. The method of claim 20, wherein the internal standard is deuterated or isotopically labeled.

22. The method of claim 1, wherein the microsampling device is encased in a cartridge designed for automation of extraction and mass spectrometric analysis.

23. The method of claim 1, wherein the microsampling device is a MITRA® tip.

24. The method of claim 1, wherein the analyte is a steroid.

25. The method of claim 24, wherein the steroid is cortisol, cortisone, progesterone, 17-hydroxyprogesterone, androstenedione, testosterone, dehydroepiandrosterone, corticosterone, deoxycorticosterone, 11-deoxycortisol, pregnenolone, 17-hydroxypregnenolone, 18-hydroxycorticosterone, 21-deoxycortisol, 25-hydroxyvitamin D₂ or 25-hydroxyvitamin D₃.

26. The method of claim 1, wherein the analyte is an opiate.

27. The method of claim 26, wherein the opiate is cis-tramadol, O-desmethyl tramadol, tapentadol, N-desmethyltapentadol, codeine, morphine, oxymorphone, norhydrocodone, oxycodone, noroxycodone, hydromorphone, hydrocodone, buprenorphine, norbuprenorphine, fentanyl, norfentanyl, 6-monoacetylmorphine (6-MAM), methadone, dihydrocodeine, naloxone, naltrexone, 6β-naltrexol, nalorphine, nalbuphine, or 2-ethylidene-1,5-dimethyl-3,3-diphenylpyrrolidine (EDDP).

28. The method of claim 1, wherein the analyte is a benzodiazepine.

29. The method of claim 28, wherein the benzodiazepine is oxazepam, temazepam, lorazepam, nordiazepam, diazepam, chlordiazepoxide, triazolam, midazolam, alprazolam, clonazepam, bromazepam, clobazam, nitrazepam, phenazepam, prazepam, medazepam, flunitrazepam, or flurazepam.

30. The method of claim 1, wherein the analyte is an anti-epileptic drug.

31. The method of claim 30, wherein the anti-epileptic drug is valproic acid, tiagabine, topiramate, levitiracetum, lamotrigine, lacosamide, ethosuximide, carbamazepine, eslicarbamazepine, 10,11-carbamazepine, phenobarbital, rufinamide, primidone, phenytoin, zonisamide, felbamate, gabapentin, or pregablin.

32. The method of claim 1, wherein the analyte is an immunosuppressant.

33. The method of claim 32, wherein the immunosuppressant is cyclosporine A, sirolimus, tacrolimus, or everolimus.

34. The method of claim 1, wherein the analyte is a barbiturate.

35. The method of claim 35, wherein the barbiturate is phenobarbitol, amobarbitol, butalbital, pentobarbitol, secobarbitol, or thiopental.

36. The method of claim 1, wherein the analyte is tamoxifen or a metabolite thereof.

37. The method of claim 36, wherein the metabolite is norendoxifen, N-Desmethyl-4-Hydroxy Tamoxifen, 4′-Hydroxy Tamoxifen, 4-Hydroxy Tamoxifen, N-Desmethyl-4′-Hydroxy Tamoxifen, N-Desmethyl Tamoxifen.

38. The method of claim 1, wherein the analyte is an oncology drug.

39. The method of claim 38, wherein the analyte is anastrozole, letrozole, or exemestane.

40. The method of claim 1, wherein the analyte is tetrahydrocannabinol (THC) or a metabolite thereof.

41. A method for determining the amount of an analyte in a sample by mass spectrometry, the method comprising:
(a) extracting an analyte from a sample collected by a microsampling device;
(b) purifying the sample by liquid chromatography;
(c) ionizing the analyte to generate one or more ions detectable by mass spectrometry; and
(d) determining the amount of the one or more ions by mass spectrometry;
wherein the amount of the one or more ions determined is used to determine the amount of analyte in the sample.

42. A method for determining the amount of an analyte in a sample by mass spectrometry, the method comprising:
(a) extracting an analyte from a sample of less than or equal to 100 μL;
(b) purifying the sample by liquid chromatography;
(c) ionizing the analyte to generate one or more ions detectable by mass spectrometry; and
(d) determining the amount of the one or more ions by mass spectrometry;
wherein the amount of the one or more ions determined is used to determine the amount of analyte in the sample.

43. The method of claim 42, wherein the method comprises extracting an analyte from a sample of less than or equal to 50 μL or less than or equal to 30 μL.
